(19) United States
(12) Patent Application Publication
(10) Pub. No.: US 2026/0137834 A1
(43) Pub. Date: May 21, 2026

(54) MESODERMAL COMPOSITIONS AND METHODS FOR THEIR USE

(71) Applicant: Children`s Medical Center Corporation, Boston, MA (US)

(72) Inventors: Martha M. Murray, Sherborn, MA (US); Nicholas John Sant, Boston, MA (US); Benedikt Lorenz Proffen, Jamaica Plain, MA (US)

(21) Appl. No.: 19/118,458

(22) PCT Filed: Oct. 5, 2023
(86) PCT No.: PCT/US2023/034594

§ 371 Date: Apr. 4, 2025

Related U.S. Application Data
(60) Provisional application No. 63/413,399, filed on Oct. 5, 2022.

(51) Int. Cl. A61L 27/36; A61L 27/24

(52) U.S. Cl. CPC A61L 27/3633; A61L 27/24

(57) ABSTRACT

The present disclosure provides biomaterials and methods for preventing and minimizing progression of cartilage and/or connective tissue damage. Also provided herein are biomaterials and methods for alleviating and/or reducing the risk for developing arthritis (e.g., osteoarthritis) associated with joint injury and/or joint surgery.

### CROSS-REFERENCE TO RELATED APPLICATIONS

This application claims benefit of priority from U.S. Provisional Application No. 63/413,399, filed on Oct. 5, 2022. The disclosure of the prior application is considered part of (and is incorporated by reference in) the disclosure of this application.

### STATEMENT AS TO FEDERALLY SPONSORED RESEARCH

This invention was made with government support under W81XWH-17-2-0016 awarded by the U.S. Department of the Army. The government has certain rights in the invention.

### TECHNICAL FIELD

This document relates to biomaterials and methods for maintaining cartilage health, preventing or reducing progression of cartilage damage, and alleviating and/or reducing the risk for developing arthritis, including early to mid-stage osteoarthritis.

### BACKGROUND

Articular cartilage in human joints often undergoes a steady process of deterioration, cumulating in osteoarthritis in the later decades of life. Small injuries typically progress to further injury. In addition, after an injury to other tissue (e.g., ligaments) within a joint, deterioration of the articular cartilage can accelerate, leading to post-traumatic arthritis within 15 years of the original injury. Tissues outside the joint can heal by forming a fibrin clot, which connects the ruptured tissue ends and is subsequently remodeled to form a scar that heals the tissue. Synovial fluid within joints naturally prevents clot formation, however. Thus, inside a synovial joint such as the knee, fibrin clots either fail to form or are quickly lysed after injury. This fibrinolytic process can result in premature degradation of the fibrin clot scaffold and disruption of the healing process for tissues within the joint or within intra-articular tissues, and progression of even minor injuries inevitably ensues.

Osteoarthritis (OA) is a disease in which the collagen within articular cartilage gradually breaks down, changing the normally smooth surface to one with multiple fissures, cracks, and defects, rendering joints stiff and painful. Osteoarthritis is extremely common; it has been estimated that 13% of men and 19% of women over the age of 60 have symptomatic osteoarthritis (Cieza et al., The Lancet, 396(10267):2006-2017, 2020). Post-traumatic osteoarthritis, or PTOA, is defined as the development of osteoarthritis in a joint that has sustained injury. PTOA can occur soon after an injury but also can remain asymptomatic for 10-20 years following injury. Joint injuries substantially increase the risk of OA, and the risk is increased with the age at the time of injury and with time from the onset of injury. For example, injuries to the anterior cruciate ligament (ACL), even when the cartilage itself is not injured in the initial event, increase the risk of developing premature OA by five times (Ajuied et al., Am J Sports Med, 42(9):2242-2252, 2014) and accelerate symptomatic OA by 15 years (Roos et al., Osteoarthritis and Cartilage, 3(4):261-267, 1995). Because so many patients who sustain joint injuries are high school and college age athletes, patients who develop PTOA are on average much younger than the un-injured, leading to “an old knee in a young patient.” This “old knee” can result in serious consequences, including a 50% increase in myocardial infarction later in life (Meehan et al., Am J Cardiol, 122(11):1879-1884, 2018), possibly due to forced relative inactivity caused by the painful joint.

Early to middle stage osteoarthritis can cause pain and disability, for which there are no disease modifying treatments. Symptomatic treatments such as oral anti-inflammatory medications, intra-articular injections of corticosteroids, and viscosupplementation have significant drawbacks. For example, oral anti-inflammatory medications carry the risk of gastrointestinal and systemic toxicity, particularly with long term use. Intra-articular injections of corticosteroids can provide pain relief, but have been demonstrated to accelerate OA progression (McAlindon and LaValley, JAMA 318(12):1185-1186 2017; and Zeng et al, Osteoarthritis and Cartilage, 27(6):855-862, 2019). Viscosupplementation with hyaluronic acid (a protein found in the normal joint) has been highly debated, as its efficacy in patients has not been demonstrated. End-stage osteoarthritis, which occurs when the cartilage has completely worn away, can be treated by total joint replacement, in which the ends of the bone(s) are removed and replaced with metal and plastic. However, joint replacement is not recommended for early to mid-stage osteoarthritis (“Surgical Management of Osteoarthritis of the Knee: Appropriate Use Criteria,” 2016; available online at aaos.org/smoakauc), because artificial joints wear out over time. Moreover, during the decades leading up to end-stage osteoarthritis, many patients experience pain and disability from the early and middle stages of the disease. Thus, there remains a significant need for treatment modalities that can relieve pain and/or slow or reverse the progression of the disease in patients with early to mid-stage OA.

### SUMMARY

This document is based, at least in part, on the development of compositions containing biomaterials (e.g., mesodermal extracellular matrix (ECM) proteins and other components) and methods for using the compositions to treat arthritis and/or reduce the risk for developing arthritis (e.g., PTOA). Accordingly, provided herein are methods and materials that can be used to treat, reduce the likelihood of developing, and/or inhibit arthritis (e.g., OA) following trauma or injury to a joint (e.g., an ACL tear, meniscal injury, minor cartilage injury, or joint surgery). The methods and materials described herein provide several advantages over the current standard of care, including limiting impact activity and subsequent treatment of chondral lesions that develop, preserving the complex morphology and architecture of the hyaline cartilage by reducing or preventing progression of injury to a damaged tissue, and restoring the biochemical makeup of cartilage at the joint site.

Thus, this document provides methods and materials that can be used to treat, prevent, or reduce the likelihood of developing OA, where the methods can include administering the materials during a routine office visit. By mixing a powder containing mesodermal ECM proteins and other components with blood and injecting the mixture into a joint affected by or at risk for OA, effects of the OA can be alleviated or even reversed. It is noted that for the purposes of this document, a composition/blood mixture is sometimes referred as a “two-part mixture” or simply a “mixture.” Injecting the mixture provided herein into a joint that has sustained an injury can prevent or delay the onset of OA that otherwise would naturally occur following the injury.

In a first aspect, this document features a composition containing a powdered extracellular matrix (ECM) component and a fluid, where the ECM component includes mesodermal proteins including collagen, and where the concentration of the powdered ECM component in the fluid is about 50 mg/mL to about 200 mg/mL. The concentration of the powdered ECM component in the fluid can be about 67 mg/mL. The concentration of the powdered ECM component in the fluid can be about 100 to about 150 mg/mL (e.g., about 133 mg/mL). The fluid can be blood. The fluid can be saline. The powdered ECM component can have an average particle size less than about 0.3 mm. The powdered ECM component can have an average particle size of about 0.1 mm to about 1 mm (e.g., about 0.3 mm to about 0.6 mm). The composition can further include a growth factor, platelets, white blood cells, stem cells, a cross-linker, a neutralizing agent, or any combination thereof. The composition can further contain calcium. The composition can be substantially free of one or more of nucleic acid, glycosaminoglycan (GAG), phospholipid, active pepsin, and active virus.

In another aspect, this document features a method for making a composition containing a fluid and a powdered ECM component containing mesodermal proteins including collagen. The method can include, or consist essentially of, providing a syringe containing the powdered ECM component, and (optionally under vacuum) drawing an amount of the fluid into the syringe such that the concentration of the powdered ECM component in the fluid is about 50 mg/mL to about 200 mg/mL. The method can include drawing an amount of the fluid into the syringe such that the concentration of the powdered ECM component in the fluid is about 67 mg/mL. The method can include drawing an amount of the fluid into the syringe such that the concentration of the powdered ECM component in the fluid is about 100 to about 150 mg/mL (e.g., about 133 mg/mL). The fluid can be blood. The fluid can be saline. The powdered ECM component can have an average particle size less than about 0.3 mm. The powdered ECM component can have an average particle size of about 0.1 mm to about 1 mm (e.g., about 0.3 mm to about 0.6 mm). The ECM composition can further contain a growth factor, platelets, white blood cells, stem cells, a cross-linker, a neutralizing agent, or any combination thereof. The composition can further contain calcium. The composition can be substantially free of one or more of nucleic acid, GAG, phospholipid, active pepsin, and active virus.

In another aspect, this document features a method for treating a mammal. The method can include, or consist essentially of, administering to a joint of a mammal that has or is at risk for developing arthritis at the joint an effective amount of a composition containing a powdered ECM component and a fluid, where the ECM component includes mesodermal proteins including collagen, and where the concentration of the powdered ECM component in the fluid is about 50 mg/mL to about 200 mg/mL. The concentration of the powdered ECM component in the fluid can be about 67 mg/mL. The concentration of the powdered ECM component in the fluid can be about 100 to about 150 mg/mL (e.g., about 133 mg/mL). The fluid can be blood. The fluid can be saline. The powdered ECM component can have an average particle size less than about 0.3 mm. The powdered ECM component can have an average particle size of about 0.1 mm to about 1 mm (e.g., about 0.3 mm to about 0.6 mm). The ECM composition can further contain a growth factor, platelets, white blood cells, stem cells, a cross-linker, a neutralizing agent, or any combination thereof. The composition can further contain calcium. The composition can be substantially free of one or more of nucleic acid, GAG, phospholipid, active pepsin, and active virus. The mammal can have an acute injury at the joint. The arthritis can be osteoarthritis. The arthritis can be post-traumatic arthritis (e.g., post-traumatic arthritis associated with an intra-articular injury or arthroscopic surgery). The intra-articular injury can be selected from the group consisting of anterior cruciate ligament tear, anterior cruciate ligament rupture, meniscal injury, and cartilage injury. The mammal can have been surgically treated for a torn, fractured, strained, bruised, or ruptured intra-articular tissue at the joint at least one day prior to the administration of the composition. The joint can be a joint of a hand, elbow, wrist, hip, knee, foot, shoulder, ankle, temporomandibular, or spine. The mammal can have an injury associated with the development of arthritis. The administering can include direct injection into the joint. The mammal can be a human.

In still another aspect, this document features a method for treating a mammal having an intra-articular tissue defect. The method can include, or consist essentially of, after visualization of the defect with an arthroscope, administering to the defect an effective amount of a composition containing a powdered ECM component and a fluid, where the ECM component includes mesodermal proteins including collagen, and where the concentration of the powdered ECM component in the fluid is about 50 mg/mL to about 200 mg/mL to the defect. The concentration of the powdered ECM component in the fluid can be about 67 mg/mL. The concentration of the powdered ECM component in the fluid can be about 100 to about 150 mg/mL (e.g., about 133 mg/mL). The fluid can be blood. The fluid can be saline. The powdered ECM component can have an average particle size less than about 0.3 mm. The powdered ECM component can have an average particle size of about 0.1 mm to about 1 mm (e.g., about 0.3 mm to about 0.6 mm). The ECM composition can further contain a growth factor, platelets, white blood cells, stem cells, a cross-linker, a neutralizing agent, or any combination thereof. The composition can further contain calcium. The composition can be substantially free of one or more of nucleic acid, GAG, phospholipid, active pepsin, and active virus. The defect can be an acute injury at the joint. The defect can be selected from the group consisting of anterior cruciate ligament tear, anterior cruciate ligament rupture, meniscal injury, and cartilage injury. The defect can be an injury associated with the development of arthritis. The administering can include direct injection into the joint. The mammal can be a human.

In another aspect, this document features a method for making a powdered composition containing mesodermal extracellular matrix (ECM) proteins. The method can include, or consist essentially of, decellularizing a tissue sample containing tissue arising from mammalian mesoderm; treating the tissue sample, before or after decellularization, with a composition containing peracetic acid; freeze-drying the decellularized tissue sample; and milling the freeze-dried tissue into a powder. The composition containing peracetic acid can contain about 0.1% peracetic acid. The method can include treating the tissue sample, before or after decellularization, for about 5 to 30 minutes with the composition containing peracetic acid. The composition containing peracetic acid can further contain hydrogen peroxide (e.g., about 1% hydrogen peroxide). The method can further include, prior to the freeze-drying, treating the decellularized tissue sample with an enzyme, thereby removing species-specific ends of collagen molecules. The method can further include treating the powder with supercritical carbon dioxide (scCO₂). The powder can have an average particle size of about 0.1 mm to about 1 mm (e.g., about 0.3 mm to about 0.6 mm). The composition can further contain a growth factor, platelets, white blood cells, stem cells, a cross-linker, a neutralizing agent, or any combination thereof. The composition can further contain calcium. The composition can be substantially free of one or more of nucleic acid, GAG, phospholipid, active pepsin, and active virus.

In another aspect, this document features a method for making a powdered composition containing mesodermal ECM proteins. The method can include, or consist essentially of, decellularizing a tissue sample containing tissue from mammalian mesoderm; freeze-drying the decellularized tissue sample; milling the freeze-dried tissue slurry into a powder; and treating the powder with scCO₂. The method can further include, prior to the freeze-drying, treating the decellularized tissue sample with an enzyme, thereby removing species-specific ends of collagen molecules. The method can further include treating the tissue sample, before or after decellularization, with a composition containing peracetic acid. The composition containing peracetic acid can contain about 0.1% peracetic acid. The method can include treating the tissue sample, before or after decellularization, for about 5 to 30 minutes with the composition containing peracetic acid. The composition containing peracetic acid can further contain hydrogen peroxide (e.g., about 1% hydrogen peroxide). The powder can have an average particle size of about 0.1 mm to about 1 mm (e.g., about 0.3 mm to about 0.6 mm). The composition can further contain a growth factor, platelets, white blood cells, stem cells, a cross-linker, a neutralizing agent, or any combination thereof. The composition can further contain calcium. The composition can be substantially free of one or more of nucleic acid, GAG, phospholipid, active pepsin, and active virus.

In another aspect, this document features a method for making a composition containing blood and a powdered mesodermal ECM component that includes collagen. The method can include, or consist essentially of, providing a syringe containing the powdered ECM component; contacting a sample of blood with an anticoagulant; drawing an amount of the blood into the syringe containing the powdered ECM component such that the concentration of the powdered ECM component in the blood is about 50 mg/mL to about 200 mg/mL; and adding a calcium chloride solution to the syringe, thereby deactivating the anticoagulant. The method can include drawing an amount of the blood into the syringe such that the concentration of the powdered ECM component in the blood is about 100 to about 150 mg/mL (e.g., about 133 mg/mL). The powdered ECM component can have an average particle size of about 0.1 mm to about 1 mm (e.g., about 0.3 mm to about 0.6 mm). The composition can further contain a growth factor, platelets, white blood cells, stem cells, a cross-linker, a neutralizing agent, or any combination thereof. The powdered mesodermal ECM component can be substantially free of one or more of nucleic acid, GAG, phospholipid, active pepsin, and active virus. The calcium chloride solution can have a concentration of about 35 mM to about 45 mM. The method can include adding the calcium chloride solution to the syringe to obtain a mixture containing a 1:9 ratio of calcium chloride solution to blood.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 includes representative images showing radiographs of guinea pig knees at six weeks after ACL transection, including a control knee (no surgery; left panel), a knee that had an ACL transection followed by a PBS injection (placebo; center panel), and a knee that had an ACL transection followed by an injection of mesodermal protein composition plus blood (treatment group; right panel).

FIG. 2 includes representative images from histology studies of the medial tibial plateau for guinea pigs at six weeks after ACL transection, including a control knee (no surgery; left panel), a knee that had an ACL transection followed by a PBS injection (placebo; center panel), and a knee that had an ACL transection followed by an injection of mesodermal protein composition plus blood (treatment group; right panel).

FIG. 3 includes representative images from histology studies of the medial tibial plateau of the treated guinea pigs at 1 week (left panel), 2 weeks (center panel), and 4 weeks (right panel) after treatment with the mesodermal protein composition/blood mixture, assessed using Toluidine Blue.

FIG. 4 includes representative images from histology studies of the medial tibial plateau of treated guinea pigs at 1 week (left panel), 2 weeks (center panel) and 4 weeks (right panel) after mesodermal protein composition/blood mixture injection, assessed using Masson's Trichrome.

FIG. 5 is a graph plotting changes in Base of Support (BOS) in animals that received no injection (Control) vs. animals that received mesodermal protein composition/blood mixture (Treatment).

FIG. 6 includes a pair of images of hydrogels containing ECM proteins, including mesodermal proteins that include collagen, generated with a powder containing “fine” particles with a diameter less than 0.3 mm (left panel) or a powder containing “coarse” particles with a diameter greater than 0.3 mm (right panel). Use of the fine particles led to more uniform distribution of the particles within the diluent.

FIG. 7 is a graph plotting the percent weight remaining after collagenase treatment of plugs of mesodermal protein hydrogels containing the indicated amounts and particle sizes of powders provided herein. *p<0.01.

FIG. 8 is a graph plotting the results of mesodermal protein gel collagenase testing, showing displacement over time. Samples containing a higher concentration of mesodermal protein powder (400 mg/3 mL) had increased resistance to degradation as compared to samples containing a lower concentration of powder (200 mg/3 mL).

FIG. 9 is a graph plotting the results of mesodermal protein gel collagenase testing, showing the normalized percentage weight remaining after collagenase treatment at 32° C. for 2 hours. The 400 mg samples retained more of their weight than the 200 mg samples. **p<0.01; ***p<0.001.

FIG. 10 includes images showing whole blood mixed with powder comprised of ECM proteins, including mesodermal proteins that include collagen, without vacuum-assist (left panels), and PRP mixed with powder with vacuum-assist (right panels). Vacuum-assist mixing provided more uniform distribution of the powder in the diluent. Magnification, 40× in the top panels and 400× in the bottom panels.

FIG. 11A is an image showing a sodium dodecylsulfate polyacrylamide gel electrophoresis (SDS-PAGE) assay of aseptically manufactured mesodermal protein powder prior to sterilization. FIG. 11B is an image of an SDS-PAGE assay of e-beam sterilized powder.

FIG. 12 is a graph plotting the dry mass fraction of collagen for pooled BEAR samples and pooled powder samples, as indicated.

FIGS. 13A-13D are graphs plotting shape scores for the indicated 200 mg/3 mL samples (FIG. 13A), cut scores for the 200 mg/3 mL samples (FIG. 13B), shape scores for the indicated 400 mg/3 mL samples (FIG. 13C), and cut scores for the 400 mg/3 mL samples (FIG. 13D).

FIGS. 14A-14D are graphs plotting plug diameters after compression testing of mesodermal protein hydrogel plugs containing 200 mg/3 mL fine powder (FIG. 14A), 200 mg/3 mL coarse powder (FIG. 14B), 400 mg/3 mL fine powder (FIG. 14C), and 400 mg/3 mL coarse powder (FIG. 14D), where the powders were generated using the indicated pH conditions.

FIGS. 15A-15C are graphs plotting the results of enzymatic degradation studies (as the percentage of initial mass) for 200 mg/3 mL and 400 mg/3 mL fine and coarse mesodermal protein powder samples, where the powders were generated using the indicated pH conditions. Results are presented for collagenase normalized to controls (FIG. 15A), collagenase digested (FIG. 15B), and controls (FIG. 15C).

FIG. 16 is a graph plotting elastic modulus for mesodermal protein hydrogels containing 200 mg or 400 mg mesodermal protein powder in 3 mL PBS.

FIGS. 17A and 17B are graphs plotting dynamic compression (FIG. 17A) and normalized dynamic compression (FIG. 17B) for mesodermal protein hydrogels prepared at concentrations of 200 mg/3 mL PBS or 400 mg/3 mL PBS and subjected to 45 compression cycles at intervals of 1 Hz (1 load cycle per second) between strains of 8-12%. For each sample, an average of the maximum (“MAX”) and minimum (“MIN”) stress response readings across the first five load cycles were used as the “START” maximum and minimum stress response, while an average of the maximum and minimum stress readings across the last five load cycles (cycles 41-45) were used to represent the “END” range of stress responses.

FIGS. 18A and 18B are graphs plotting stress relaxation for mesodermal protein hydrogels containing 200 or 400 mg powder in 3 mL PBS over a 10 minute period (FIG. 18A), or extrapolated stress relaxation to 100 minutes (FIG. 18B).

FIG. 19 is a graph plotting Poisson's Ratio for mesodermal protein hydrogels containing 200 mg powder or 400 mg powder in 3 mL PBS.

FIG. 20 is a graph plotting total soluble collagen content compared to untreated control. Mesodermal tissues were treated for 1, 5, and 10 minutes (doubled treatment times for hydrogen peroxide) as indicated by the three bars from left to right. Groups with collagen contents significantly lower than the control are denoted based on p-value: *p<0.05, **p<0.001, ++p<0.0001.

FIG. 21 is a graph plotting total GAG content compared to untreated control. Mesodermal tissues were treated for 1, 5, and 10 minutes (doubled treatment times for hydrogen peroxide) as indicated by the three bars from left to right. Groups with GAG contents significantly lower than the control are denoted based on p-value: ++p<0.0001.

FIG. 22 is a graph plotting Gelation Scores as compared to untreated control. Mesodermal tissues were digested in pepsin to form slurries before being placed in a 37° C. incubator for 30 seconds. Groups with significantly worse gelation scores than the control are denoted based on p-value: ++p<0.0001. Gelation Scoring Guideline: 0, no maintenance of mold shape; 1, maintains less than half of the original central height when gel unmolded onto Petri dish; 2, maintains over half of the original height when gel unmolded onto Petri dish; 3, maintains mold shape with rounding of corners, not stable when cut into; 4, maintains mold shape with rounding of corners, stable when cut; 5, maintains mold shape with crisp corners which are maintained when gel is cut.

FIG. 23 is an image showing an SDS-PAGE analysis used to compare protein breakdown due to chemical pretreatment. Differences in banding were minimal, with no major observable differences in protein breakdown. Major Bands represent: (a) Type I collagen alpha polypeptides (b) alpha polypeptide dimers and (c) alpha polypeptide trimers (collagen triple molecule).

FIG. 24 is a graph plotting collagen concentration after treatment with various PAA protocols.

FIG. 25 is a graph plotting the GAG content and GAG/Collagen ratio with various PAA treatment protocols.

FIG. 26 is an image of a representative western blot for ECM-derived powder samples treated as indicated in TABLE 12. M, standard protein ladder; lane 1, 0.1% PAA 1 hour; lane 2, 0.2% PAA 1 hour; lane 3, 0.1% PAA 18 hours; lane 4, 0.2% PAA 18 hours; C, control; lane 5, 7.5% HP 10 minutes; lane 6, 1% CIP-100 10 minutes; lane 7, SPOR-KLENZ® 10 minutes; lane 8, 0.2% PAA 10 minutes.

FIG. 27 is a graph plotting gelation data for ECM-derived powder samples treated with the indicated PAA protocols.

FIG. 28 is an image from SDS-PAGE using the ECM-derived powder protein composition.

FIG. 29 is a graph plotting collagen content in samples sterilized using the indicated methods. Data are mean±standard deviation.

FIG. 30 is a graph plotting GAG content in samples sterilized using the indicated methods. Data are mean±standard deviation.

FIG. 31 is a graph plotting DNA content in samples sterilized using the indicated methods. Data are mean±standard deviation.

FIG. 32 is a graph plotting phospholipid content in samples sterilized using the indicated methods. Data are mean±standard deviation.

FIG. 33 is a graph plotting residual pepsin activity in samples sterilized using the indicated methods. Data are mean±standard deviation.

FIG. 34 is an image of an SDS-PAGE gel for samples sterilized with the indicated methods. A standard ladder is provided in the left lane and a collagen reference is provided in the far-right lanes.

FIG. 35 includes a pair of graphs plotting the percent weight remaining for controls and treatment groups after enzymatic degradation (left) and normalized treatment/control percent weight remaining for e-beam and supercritical carbon dioxide (scCO₂) powder derived gels (right).

FIG. 36 is a graph plotting elastic modulus for samples containing the indicated amounts of mesodermal ECM-derived powder and sterilized by the indicated methods. Mean±standard deviation.

FIG. 37 includes images showing, from left to right, a frontal view and lateral views after opening of a pig knee joint and removal of synovium, menisci from the joint, followed by a central histologic section of the ACL at low and high magnification.

FIG. 38 is a graph plotting ACL volume in pigs after ACL transection followed by treatment with BEARD scaffold or mesodermal protein powder.

FIG. 39 is a graph plotting ACL histologic scores in pigs after ACL transection followed by treatment with BEAR® scaffold or mesodermal protein powder.

FIG. 40 includes images showing, from left to right, a dorsal view and lateral views after opening of a sheep shoulder joint, followed by a central histologic section of the RCT at low and high magnification.

FIG. 41 is a graph plotting RCT volume in sheep after RCT transection followed by treatment with suture only, mesodermal protein powder, mesodermal protein sheet, or BEAR® scaffold.

FIG. 42 is a graph plotting RCT histologic scores in sheep after RCT transection followed by treatment with suture only, mesodermal protein powder, mesodermal protein sheet, or BEAR® scaffold.

### DETAILED DESCRIPTION

This document provides methods and materials, including compositions, that can be used to treat and/or reduce the risk for developing arthritis. In general, the compositions provided herein can contain proteins and other components derived from mesodermal ECM (e.g., proteins such as collagen and fibrillin). The composition also can contain components such as laminin, salt, and/or calcium. The methods provided herein can be used to, for example, fill cartilage defects, treat OA (e.g., by reversing osteoarthritic gait changes in subjects with early to mid-stage osteoarthritis), and stop the progression of post-traumatic osteoarthritis after a joint injury, even when administration of a composition provided herein occurs a significant time after the initial injury.

The compositions of mesodermal ECM (e.g., proteins and other components) can be prepared as powders. Briefly, a powder can be prepared by (1) decellularizing a tissue that arose from mammalian mesoderm (the “tissue”), (2) enzymatically digesting the decellularized tissue. (3) freeze-drying the decellularized tissue, and (4) milling the freeze-dried tissue into a powder.

The composition can be administered, in some cases, via direct injection from a syringe. In general, the steps to administer a composition provided herein can include: (1) adding the powdered composition to a syringe, or providing a syringe containing the powdered composition, (2) drawing blood into the syringe containing the composition, (3) mixing the blood with the composition, and (4) injecting the blood/composition mixture into a joint. In some cases, the powdered composition can be hydrated with an aqueous salt solution or water before drawing the blood into the syringe.

This document also provides articles of manufacture having a compartment housing the powdered composition, optionally a compartment housing an aqueous solution (the “hydrating solution”), optionally a device for mixing the composition and the optional hydrating solution with blood (e.g., autologous blood from a patient), where the mixing device can be controlled from outside of container, and optionally an internal mixing chamber that is large enough to incorporate the powder, the optional hydrating solution, and the blood.

In a first aspect, this document provides compositions containing a powder, optionally combined with a fluid (e.g., blood or another fluid containing blood cells). Thus, in some cases, the compositions provided herein can be two-part mixtures, where the first part contains powdered ECM components (e.g., proteins) that are found in mesodermal tissues. The mesoderm is the mammalian embryonic layer from which the limbs develop, and gives rise to multiple tissues. Proteins typically found in the mesoderm include collagen, elastin, fibrillin, and other glycoproteins. The powder in the compositions provided herein (also referred to as the “powder compositions”) can be made by processing tissues that arise from the mesoderm to isolate these proteins. The second part can include, for example, autologous blood from a patient to be treated.

The first part of a composition provided herein can be derived from tissues that arise from the mammalian mesoderm and can contain, for example, collagen and/or fibrillin. Tissues that arise from mammalian mesoderm include, for example, muscle (e.g., skeletal muscle and smooth muscle), connective tissue (e.g., skin, bone, ligament, tendon, bursae, synovium, loose connective tissue, fascia, separating fascia, and investing/subcutaneous fascia), blood vessels (e.g., the aorta, vena cava, arteries, veins and capillaries), bone, cartilage (e.g., articular cartilage, fibrocartilage, hyaline cartilage, and elastic cartilage), kidney, adipose tissue, and the urogenital organs. In some cases, the powder composition can be derived from an elastic tissue (e.g., ligamentum nuchae, arteries, the dermis of the skin, loose connective tissue, adipose tissue, and lung). Such tissues can be useful for deriving the powder composition because they have high concentrations of collagen and, optionally, fibrillin.

The powder portion of the compositions provided herein can be made by decellularizing tissue (e.g., elastic tissue) and breaking down the original structure of the tissue into a fluid form. As noted above, the tissue can be from any of a variety of sources, including blood vessels, the ligamentum nuchae, fascia, bursae, synovial sheaths, skeletal muscle, and/or smooth muscle. Moreover, the tissue can be derived from human or non-human animal sources, including bovine, porcine, caprine, or other mammalian species. The tissue can be from animals that are skeletally mature, or from animals that have growth remaining. For example, in some cases, the tissue can be from animals between one week and one year of age, between three months and six months of age, or less than six months of age. In some cases, the tissue (e.g., elastic tissue) can come from recombinant technology or other manufacturing methods for manufacturing proteins.

In some cases, the tissue can be treated before additional processing with compounds designed to remove bacterial, fungal, and/or viral contamination. Such treatment can include the use of chemicals such as sodium hypochlorite, peracetic acid, hydrogen peroxide, antibiotics, and/or acetic acid. In some cases, the treatment can include physical washing of the tissue, exposure to high or low pH, ultraviolet light, heat, steam, gamma irradiation, or electron beam irradiation, or treatment with gas (e.g., ethylene oxide or supercritical CO₂) or induced free oxygen radicals to remove or inactivate infectious compounds that may have been introduced to the tissue during its procurement. For example, tissues that arise from mammalian mesoderm can be sterilized using supercritical CO₂ for an appropriate length of time (e.g., about 2 to about 16 hours, about 3 to about 14 hours, about 4 to about 12 hours, about 6 to about 10 hours, about 2 to about 4 hours, about 4 to about 6 hours, about 6 to about 8 hours, about 8 to about 10 hours, about 10 to about 12 hours, about 12 to about 14 hours, about 14 to about 16 hours, or about 2, 3, 4, 5, 6, 7, 8, 9, 10, 11, 12, 13, 14, 15, or 16 hours). Such tissue pretreatments to remove or reduce bioburden can be used alone or in various combinations. In some cases, hydrogen peroxide, peracetic acid (PAA), or a combination thereof can be used to treat the tissue before proceeding with processing into the powder composition.

When a tissue sample is treated with peracetic acid and/or hydrogen peroxide, the treatment can take place for any appropriate length of time, and the peracetic acid and/or hydrogen peroxide can be used at any appropriate concentration. In some cases, for example, a tissue sample can be treated with peracetic acid, hydrogen peroxide, or a combination thereof for 5 to 30 minutes (e.g., 5 to 10 minutes, 10 to 15 minutes, 15 to 20 minutes, 20 to 25 minutes, 25 to 30 minutes, about 5 minutes, about 10 minutes, about 15 minutes, about 20 minutes, about 25 minutes, or about 30 minutes). In some cases, a tissue sample can be treated with a composition containing about 0.01% to about 1% peracetic acid (e.g., about 0.01% to about 0.05%, about 0.05 to about 0.1%, about 0.1 to about 0.15%, about 0.15 to about 0.25%, about 0.25 to about 0.5%, about 0.5 to about 1%, about 0.01%, about 0.05%, about 0.1%, about 0.2%, about 0.5%, or about 1% peracetic acid). In some cases, a tissue sample can be treated with a composition containing about 0.1 to about 10% hydrogen peroxide (e.g., about 0.1 to about 0.2%, about 0.2 to about 0.5%, about 0.5 to about 1%, about 1 to about 2%, about 2 to about 5%, about 5 to about 10%, about 0.1%, about 0.5%, about 1%, about 2%, about 5%, or about 10% hydrogen peroxide). In some cases, a tissue sample can be treated with a composition containing about 0.01% to about 1% (e.g., about 0.1%) peracetic acid and about 0.01 to about 10% (e.g., about 1%) hydrogen peroxide.

In general, the powder compositions provided herein can contain proteins expressed early in development of the mesoderm. These proteins can signal the stem cells in the blood and in the tissue having a defect to be treated, such that they come and develop new tissue in the defect. The strategy of recruiting native cells from the surrounding tissue to come into a provided scaffold of proteins and fill in a tissue defect is very different from implanting tissue-specific mature cells within a mature matrix into a defect.

Collagens are the main structural proteins in the ECM of connective tissues in the body. Collagen is the most abundant protein in mammals, and is found in developing, healing, and mature tissues, and in the mesoderm of mammalian embryos. The six most common types of collagen are Type I, II, III, IV, V. and VI. Type I is found in mature and healing skin, tendon, blood vessels, organs, discs, and bone. Type I collagen is made up of two collagen alpha-1(I) chains and one collagen alpha-2(I) chain, thus there would be a 1:1 ratio of Type I collagen molecules and the collagen alpha-2(I) chain in a proteomic analysis such as mass spectrometry. Type II collagen is the main form of collagen found in articular cartilage. Type III collagen is most commonly found in healing fibrous tissues, including healing ligaments, tendon, and skin, and is found with Type I collagen in those cases. Type IV collagen is found in the basal lamina of tissues, and Type V collagen is found on cell surfaces and in hair and placenta. Type VI collagen is found in the extracellular matrix of skeletal muscle. In mass spectrometry, proteins can be detected using peptides unique to those proteins and determining a spectral count. In some cases, a composition provided herein can contain between 10% and 90% (e.g., between 10% and 50%, between 10% and 40%, or between 10% and 20%) type I collagen when quantified by mass spectrometry using normalized spectral counting for the collagen alpha-2(I) chain. The remaining proteins can, in some cases, include fibrillins and/or other types of collagen.

The powder compositions provided herein optionally can contain fibrillin. Fibrillin is a key glycoprotein of the mesoderm and is essential for the formation of elastic fibers in connective tissue. Since elastic fibers are a key component of tissues such as articular cartilage, skin, blood vessels, ligaments, tendons, bone, and discs, the presence of fibrillin in the compositions provided herein may assist with restoration of these tissues. Three forms of fibrillin are known in mammals: fibrillin-1, fibrillin-2, and fibrillin-3. Fibrillin-1 and fibrillin-2 are both thought to play key roles in the development of elastic tissues, while fibrillin-3 is largely found in the brain. Fibrillin-1 is thought to provide force-bearing structural support in tissues, while fibrillin-2 is thought to guide elastogenesis.

In some cases, the powder compositions provided herein can contain fibrillin-1 and fibrillin-2. Again, the proteins in a composition containing multiple proteins can be determined using mass spectrometry. In some cases, the spectral count of fibrillin-1 in a composition can be between 1% and 99% (e.g., between 10% and 90%, between 10% and 50%, or between 10% and 25%) of the total spectral count of the powder composition. The remaining proteins can include, for example, members of the collagen family and other fibrillins. In some cases, a powder composition can contain similar spectral counts for fibrillin-1 and Type I collagen as measured by the spectral count of collagen alpha-2(I) chain. In some cases, a powder composition can contain between 2-fold and 20-fold more collagen than fibrillin protein, or between 2-fold and 4-fold more type I collagen than fibrillin when determined by spectral count. In some cases, the spectral count of fibrillin-1 of a powder composition can be 15 to 25% of the total spectral count, and the spectral count of the collagen alpha-2(I) chain is 10 to 20% of the total spectral count of the powder composition.

In some cases, a powder composition can contain mostly collagen and fibrillin, such that collagen and fibrillin make up between 10% and 100% of the composition. In some cases, collagen and fibrillin can make up between 20% and 90% of the composition (e.g., between 30% and 80% of the composition), or collagen and fibrillin can make up at least 50% of the composition. In some cases, collagen can make up at least 40% of the composition, while fibrillin can make up at least 20% of the composition.

In addition to proteins, the powder compositions provided herein can contain ingredients such as, without limitation, one or more laminins, salts, growth factors, cross-linkers, neutralizing agents, or any combination thereof. For example, laminins are a major component of the basal lamina, and are a key part of the protein network foundation for many organs. Laminin can influence cell differentiation, migration, and adhesion. In some cases, the spectral count of laminin (as determined by mass spectrometry) can be between 0.1 and 2% of the total spectral count of the powder compositions provided herein.

Salts, including sodium chloride, can be useful in a composition when the powder is hydrated, as the salts can create a slurry of the powder that has an osmolarity similar to that seen in blood so that when the blood is added to the hydrated powder, the cells in the blood remain the same size. If blood cells are added to a solution with low osmolarity, they might expand to the point where they burst, thus preventing their ability to function in a physiologic way to stimulate tissue healing. In some cases, dry salts can be added to a powder composition to ensure the osmolarity of the hydrated powder is similar to that of blood, and ranges between about 200 and about 350 mOsm (e.g., about 280 to about 320 mOsm). In some cases, salt can be added to a powder composition in a liquid form, such as in phosphate-buffered normal saline or unbuffered normal saline, or saline at a concentration other than normal (e.g., half normal saline or a salt solution containing other concentrations of salt or other solutes). In some cases, the osmolarity of a powder when hydrated with physiologically buffered saline can be 280 to 320 mOsm.

In some cases, the compositions provided herein can contain calcium or a calcium salt (e.g., calcium chloride). The calcium chloride can be in solid or liquid form. For example, the salt can be added in solid form to a powder composition prior to mixing with another liquid (e.g., saline, water, or blood), or the salt can be added in liquid form to a powder or hydrated powder composition. In some cases, a composition can contain calcium in a concentration sufficient to reverse the effects of an anticoagulant that works by sequestering calcium (e.g., sodium citrate or acid-citrate dextrose). The calcium also can be included in a solution containing a salt. When a salt and calcium-containing solution is added to a powder composition, the osmolarity of the resulting combination can be close enough to the physiologic osmolarity of blood that the blood cells are not adversely affected by the mixing, and the calcium concentration can be sufficient to reverse the effects of a calcium-sequestering anticoagulant. In some cases, the combination of powder, calcium, salt, and water prepared just prior to adding a patient's blood can have an osmolarity in the range of about 250 to about 350 mOsm. In some cases, a calcium solution can be mixed with a salt solution prior to adding to the powder composition, and the resulting combination can have (1) a concentration of calcium sufficient to reverse the effects of the calcium-sequestering anti-coagulant that has been added to the autologous blood to be mixed with the combination, and (2) an osmolarity from about 250 to about 350 mOsm (e.g., about 280 to about 320 mOsm). In some cases, a solution containing calcium chloride at a concentration of about 30 mM to about 50 mM (e.g., about 35 mM to about 45 mM, about 38 to about 43 mm, about 35 mM, about 36 mM, about 37 mM, about 38 mM, about 39 mM, about 40 mM, about 41 mM, about 42 mM, about 43 mM, about 44 mM, or about 45 mM) can be used. A solution containing calcium chloride can be combined with anticoagulated blood at any appropriate ratio. For example, a solution containing about 35 mM to about 45 mM calcium chloride can be combined with blood at a ratio of about 1:7, about 1:8, about 1:9, about 1:10, about 1:11, or about 1:12 calcium chloride solution to blood.

In some cases, the compositions provided herein can be substantially free of one or more of nucleic acid, glycosaminoglycan (GAG), phospholipid, active pepsin, and active virus. A composition that is “substantially free” of a particular component is a composition that contains less than about 1% (e.g., less than 0.8%, less than 0.5%, or less than 0.1%) by weight of that component.

This document also provides methods for making the compositions disclosed herein—including the powder compositions and the two-part compositions containing a powder and a fluid (e.g., blood). In general, a method for making a powder can include: (1) decellularizing a tissue that arose from mammalian mesoderm (the “tissue”); (2) treating the tissue with an enzyme to remove the species-specific ends of collagen molecules, yielding slurry; (3) freeze-drying the tissue slurry; and (4) milling the freeze-dried tissue slurry into a powder.

Decellularizing the tissue can be accomplished using any suitable agent(s). For example, a tissue can be decellularized with one or more detergents, enzymes, salts, or any other appropriate physical or chemical method, to yield an ECM. In some cases, the decellularization method can reduce the DNA content of the tissue such that the DNA content in the final powder composition is less than about 20,000 ng/g powder. The decellularization method also can reduce the phospholipid content of the tissue, such that the phospholipid concentration of the final powder composition is less than about 3000 μM/g of powder. In some cases, the DNA content of the powder can be less than about 50,000 ng/gm of powder. In some cases, the phospholipid content of the powder can be less than about 300 μM/g. After the decellularization treatment, the tissue can be washed (e.g., with water or an aqueous solution such as saline) to remove residual chemicals, enzymes, or excess salts.

In some cases, the decellularized tissue (the ECM) can be treated with pepsin in an acidified solution, typically at a pH below 5, to create a slurry of proteins in a fluid form. Pepsin another enzyme can remove the telopeptides at the ends of the collagen molecules to produce atelocollagen, which is a low-immunogenic derivative of collagen obtained by removal of N- and C-terminal telopeptide components that can induce antigenicity in humans. In some cases, therefore, pepsin is used to cleave the collagen present in the tissue and convert it to atelocollagen. It is noted, however, that any other enzyme capable of digesting proteinaceous tissue (such as, without limitation, collagenase, trypsin, or elastase) can be used to create a protein slurry. After digestion with pepsin or another suitable enzyme, the resulting protein slurry can be neutralized to a pH greater than 8.5 to inactivate the pepsin or other enzyme(s). Neutralization can be achieved by adding a base (e.g., NaOH) or a buffer (e.g., a phosphate buffer). In some cases, the protein slurry can have a basic solution added to bring the pH of the solution to a pH greater than 7.5, and then have an acid or buffer added to bring the pH of the solution back to 7.0-7.4. Such a final pH for the slurry can be particularly useful, since when the slurry is lyophilized and milled to create a powder, the powder can be readily mixed with blood without changing the pH of the blood. Further, if the powder is hydrated before being mixed with blood, the hydrated powder solution will have a pH close to the physiologic pH of the blood that will be added to the hydrated powder. In some cases, after treatment at an acid pH with pepsin, the tissue slurry can be neutralized to a pH between 7.5 and 8.5 and then brought back to a pH between 6.5 and 8.5 by the addition of acid. In some cases, the tissue slurry can be kept at a temperature below 4° C. during this process. In some cases, a decellularized tissue can be enzymatically treated with pepsin at a pH below 4.0, the resulting slurry can be brought to a pH greater than 8.5 using a based (e.g., NaOH), and the slurry then can be neutralized to a pH between 7.0 and 7.4 prior to lyophilization.

The step of freeze-drying (lyophilizing) the mesodermal ECM composition in slurry form typically consists of bringing the slurry down to a temperature at which the water within the slurry is frozen, and then applying a vacuum to the frozen slurry to sublimate the water from the composition, leaving a dry, porous sheet. The dried sheet can then be made into a powder by milling, grinding, blending or any other appropriate method. In some cases, for example, a lyophilized ECM sheet can be made into a powder by milling while cooling the composition, keeping the temperature of the composition below about 4° C. The particles within the resulting powder can have any appropriate size. For example, the particles can have an average diameter of about 0.1 mm to about 1 mm (e.g., about 0.1 mm, about 0.2 mm, about 0.3 mm, about 0.4 mm, about 0.5 mm, about 0.6 mm, about 0.7 mm, about 0.8 mm, about 0.9 mm, about 1 mm, less than about 0.3 mm, less than about 0.4 mm, less than about 0.2 mm, about 0.1 to about 0.2 mm, about 0.2 to about 0.3 mm, about 0.3 to about 0.4 mm, about 0.4 to about 0.5 mm, about 0.5 to about 0.6 mm, about 0.6 to about 0.7 mm, about 0.7 to about 0.8 mm, about 0.8 to about 0.9 mm, about 0.9 to about 1 mm, about 0.3 to about 0.5 mm, about 0.4 to about 0.6 mm, about 0.3 to about 0.6 mm, about 0.4 to about 0.7 mm, or about 0.5 to about 0.8 mm). In some cases, the powdered composition can be loaded into a syringe, which can optionally be packaged and/or sterilized prior to use. Alternatively, the powdered composition can be placed in any other appropriate type of container for storage or use.

It is noted that in some cases, the composition provided herein can be prepared in a liquid, semi-liquid, or slurry suspension in which a mesodermal ECM powder is combined with a liquid solution, by mixing the powder with a fluid that does not contain cells. Such a “hydrated composition” can be prepared by mixing an amount of the powdered composition with an amount of a hydrating solution. The hydrating solution can be, for example, water or a solution that contains a salt such as sodium chloride (e.g., normal saline, normal phosphate buffered saline, or a mixture of water and saline). In some cases, the hydrating solution can contain calcium, glucose, phosphate, other salts, and/or an anesthetic agent. For example, the hydrating solution can contain calcium at a concentration sufficient to reverse the effects of a calcium-reversible anticoagulant, such as sodium citrate or acid-citrate-dextrose. In some cases, the hydrating solution can be a solution that contains blood cells or proteins, including plasma (e.g., autologous or non-autologous plasma).

In some cases, the proteins in the mesodermal ECM powder compositions provided herein can self-assemble and form a gel within a certain length of time (e.g., about 20 minutes) after mixing with a hydrating solution or blood when the mixture is at a suitable temperature (e.g., between about 30° C. and about 39° C.). The term “self-assemble” as used herein refers to the ability of a composition to change from a viscous liquid to a solid material that can be removed from its container or cut with a knife and not lose its shape. In some cases, proteins in a hydrated mesodermal ECM powder composition can self-assemble into a gel within about five minutes of mixing with a hydrating solution or blood cells (e.g., red blood cells, white blood cells, platelets, or platelet-rich plasma). In some cases, a composition provided herein can self-assemble and form a gel within about ten minutes of mixing with water or blood cells when the mixture is at about 32° C. In some cases, when a composition is hydrated such that the osmolarity of the resulting solution is between about 270 and 330 mOsm and the pH is between 6.8 and 7.4, the composition can self-assemble at a temperature close to that of the interior of the human knee.

This document also provides articles of manufacture that include a powdered composition described herein, where the powdered composition is in any appropriate container. In some cases, the powdered composition can be in a container having a single chamber that contains the composition. Alternatively, the powdered composition can be stored in a first chamber of a container that also has a second chamber for holding a hydrating solution (e.g., water, saline, a calcium solution, or another material used for hydrating the composition prior to adding the blood component), or the powdered composition can be in a first container and the hydrating solution can be in a second container within the kit. The containers or chambers can be connected in the kit, or can be separate but connectable (e.g., at the time of administration of the composition). In some cases, the powdered composition can be in a first syringe, and when present, the hydrating solution can be in a second syringe or other container. In some cases, a kit can include a first syringe containing the composition, a second syringe containing water, and a connector. Prior to administration, the two syringes can be removed from the kit and connected via the connector. In some cases, the syringe plungers can be manipulated from outside the chambers containing the powder and the solution, to move the hydrating solution into the syringe containing the powder, and then move the resulting suspension back and forth between the syringes to facilitate uniform mixing. While syringes can be particularly useful, it is to be noted that any appropriate vessel, connecting device, and mixing mechanism can be used.

Further, the container can be configured to assist with mixing of the blood with the powder or the hydrated composition. The container can be larger than the volume of the composition and the optional hydrating material, such that it also can accommodate up to 20 cc of autologous blood. In some cases, the container can house a mechanism to combine the composition (the powder with or without the hydrating solution) and the blood at the point of care. Suitable mechanisms include, without limitation, a syringe connector such as a Luer lock, a membrane that can be ruptured between the chambers to allow for mixing by externally rocking or shaking the container, and an internal collapsible augur that can be controlled by an external plunger. In some cases, as described herein, autologous blood can be drawn into a sterile syringe, and the syringe containing the blood then can be connected to a syringe containing the powder or the hydrated composition. The plungers of the syringes can be used to mix the resulting composition prior to administration of the mixture to a patient.

The composition, in either the hydrated or powder state, can be rendered sterile prior to administration to a patient. When a hydrating fluid is to be combined with a powder composition prior to the addition of blood, the combination of the powder and the hydrating fluid can be rendered sterile prior to administration to a patient. This can be accomplished by individually sterilizing each component prior to placement into a sterile container, sterilizing each component in separate containers and mixing using sterile technique, or sterilizing both components in one sterile container, with or without separating compartments. Suitable methods of sterilization that can be used to reduce the bioburden of the composition include, without limitation, radiation (e.g., gamma irradiation or electron beam irradiation), sterilization using free oxygen radicals, gas sterilization (e.g., with ethylene oxide or supercritical CO₂), and ultraviolet radiation. When radiation is used, it can be used at a dose between 15 and 25 kGy (e.g., between 17.5 and 22.5 kGy). The dose of radiation typically is such that the manufacturing process reduces both bacterial and viral loads of the tissue to a sterility assurance level of 10⁻⁶. In some cases, a composition provided herein can be sterilized in its final packaging using supercritical CO₂ or electron beam irradiation at a dose of 20 kGy.

Before administration to a patient, a powdered mesodermal ECM composition provided herein (also referred to as a “powder” or a “powdered composition”) is combined with blood or another fluid (e.g., a processed blood sample) that contains blood cells (e.g., red blood cells). In some cases, the blood is autologous blood from the patient to be treated. In some cases, however, the blood can be from a third-party donor, including from another human or animal donor. If blood from another donor is used, it may be processed prior to administration to reduce the antigenicity of the blood for the recipient.

The blood can be collected ahead of time (e.g., in a lab) or can be drawn on-site during an office visit, processed if desired, and brought to the administering clinician for mixing/injection. In some cases, whole blood can be used, but in other cases, a fluid (e.g., a processed blood sample) containing blood cells (e.g., red blood cells) can be used, where the fluid is not whole blood. The fluid can contain plasma proteins, platelets, and/or white blood cells, in combination with red blood cells. In some cases, the fluid also can contain precursor or stem cells, particularly those normally found in circulating blood. The red blood cells can be present in the fluid in a concentration similar to that found normally in mammalian (e.g., human) blood, or the blood may be processed such that the concentration of red blood cells is greater or less than that of normal mammalian blood. In some cases, the concentration of red blood cells can be within about 10% of the concentration found in the circulating blood of the patient to whom the composition is to be administered.

In some cases, a blood sample drawn from a patient can be processed prior to mixing with the powder or hydrated powder composition. For example, a blood sample can be processed by centrifugation, filtration, and/or passing through a cell separation column to isolate certain types of cells in the blood. In some cases, serial centrifugation can be used to isolate platelets, which then can be resuspended in plasma at a higher concentration than would be found in unprocessed blood (thus generating platelet-rich plasma, or “PRP”). The concentration of other types of cells, including white blood cells and specific subpopulations of white blood cells, including stem cells, also can be carried out to increase the effect of the final composition on the tissue defect being treated. In some cases, blood that contains physiologic levels of blood cells and plasma proteins (whole blood) can be used.

Any suitable phlebotomy method can be used to obtain a blood sample from a mammal (e.g., a human patient). Typically, blood can be obtained from a mammal using a needle. The needle can have any suitable size, typically 14 gauge to 22 gauge (e.g., 16 gauge to 20 gauge). In some cases, the needle can be an 18 gauge needle, which generally is the smallest size that does not cause significant damage to cells in the blood. The skin of the mammal from which the blood is to be drawn can be cleaned with a preparation to eliminate bacteria from the skin. A tourniquet can be used proximal to the site of the blood draw to increase the size and visibility of the vessel from which the blood sample will be obtained. A needle (e.g., 18 gauge or larger) can be placed through the skin and into the blood vessel, and the blood can be removed through the needle into a syringe or tube. In some cases, the blood can be drawn into a tube that initially has a vacuum to pull the required amount of blood into the tube.

In some cases, blood from a mammal (e.g., a human patient) can be drawn into a syringe or tube that contains an anticoagulant (e.g., a liquid or solid anticoagulant). The anticoagulant may be one that can be reversed by the addition of calcium, such that the anticoagulation is reversed when the blood is added to the powdered composition when the powdered composition contains calcium. Examples of this type of anticoagulant include, without limitation, sodium citrate and acid-citrate-dextrose. In some cases, the blood can be drawn into a tube containing liquid acid-citrate-dextrose, where the volume of blood is 10 times greater than the volume of acid-citrate-dextrose. In some cases, blood can be drawn into a syringe, tube, or other vessel containing an amount of a calcium-chelating anticoagulant (e.g., acid-citrate-dextrose) sufficient to prevent coagulation of the blood. In some cases, when a calcium-chelating anticoagulant is used, a solution of calcium can be added to the powdered ECM composition or the blood just before combining the blood with the ECM composition, to reverse the anti-coagulant and allow the blood to clot. If no anticoagulant is used, the blood can be mixed with the powdered composition (or the hydrated powder composition) within five minutes of venipuncture. The blood can be kept at room temperature until use.

The blood and powdered mesodermal ECM composition can be combined in any appropriate amounts. In some cases, a composition provided herein can be a hydrogel containing a mesodermal ECM powder and blood, where the powder is present at a concentration of about 50 mg/mL to about 200 mg/mL (e.g., about 50 to about 100 mg/mL, about 100 to about 150 mg/mL, or about 150 to about 200 mg/mL). In some cases, for example, 200 mg of a powdered composition can be mixed with 3 mL of blood, so the concentration of the powder in the mixture is about 67 mg/mL. In some cases, 400 mg of a powdered composition can be mixed with 3 mL of blood, so the concentration of the powder in the mixture is about 133 mg/mL.

Further, the blood and powdered mesodermal ECM composition can be combined by any appropriate method. For example, in some cases, blood can be poured into a container holding a powdered mesodermal ECM composition, and mixed by stirring. In some cases, blood can be drawn into a syringe containing a powdered mesodermal ECM composition by actuation of the plunger of the syringe. In some cases, a vacuum can be generated in a first syringe containing a powdered mesodermal ECM composition, and a second syringe containing blood can be connected to the first syringe (e.g., via a Luer lock or connector having a valve). When the valve is opened such that the chambers of the first and second syringes are in fluid communication with each other, the blood can be pulled into the first syringe due to the vacuum. Moreover, the vacuum can facilitate mixing of the powder with the blood.

In some cases, a powdered mesodermal ECM composition can be hydrated with an acellular fluid (“hydrating solution”) prior to mixing with blood (or another fluid containing blood cells). The hydrating fluid can contain, for example, water and one or more of sodium, chloride, calcium, phosphate, glucose, and/or any other molecules typically found in injectable saline or phosphate buffered saline. The ratio of composition to fluid used to hydrate the composition can be such that when the hydrated powder is then mixed with blood, the resulting combination forms gel-like matrix.

In some cases, a powdered composition can be combined with a hydrating fluid to distribute the powder particles prior to combining with blood. The ratio of powder to water can range from about 100 mg powder:0.1 mL water to about 100 mg powder:5 mL water. For example, the ratio can be 100 mg powder:0.5 mL water or saline. In some cases, the fluid can contain calcium in a level that would be sufficient to reverse the effect of a calcium binding anticoagulant, such as sodium citrate or acid-citrate-dextrose. In some cases, 600 mg of a powder composition can be mixed with 3 mL of water, and the hydrated composition is then mixed with 3 mL of autologous blood drawn without an anticoagulant (resulting in a composition having a powder concentration of 100 mg/mL). In other cases, 600 mg of a powder composition can be combined with 3 mL of water containing calcium, and the hydrated composition then can be mixed with 3 mL of blood anticoagulated with acid-citrate-dextrose (again resulting in a composition having a powder concentration of 100 mg/mL).

This document also provides methods for treating, preventing, or reducing the likelihood of development or progression of arthritis (e.g., OA). In some cases, the methods provided herein can be carried out during a routine office visit, or in a room designed specifically for such procedures (including injections), or in an operating room. The methods can be performed after topical, local, oral, regional, or systemic anesthesia or analgesia has been administered to the patient, or with no anesthesia. The methods can include delivery of a composition provided herein be without visualization of the defect to be treated, or with visualization directly by eye or using imaging techniques such as ultrasound, MRI, x-ray, arthroscopy, or computed tomography (CT) scanning. In some cases, for example, a method provided herein can be carried out with local anesthesia in an office setting.

By injecting a composition containing ECM proteins derived from tissues of mesodermal origin and blood into a joint, the effects of OA can be reduced or reversed. Combining a powdered composition provided herein with blood from a patient can stimulate formation of copolymers of proteins from the powder (e.g., collagen and fibrillin) and proteins in the patient's blood (e.g., fibronectin), which can bind to exposed collagen in cartilage that has damage from early OA, providing a provisional scaffold for cartilage healing. The solidified gel-like material can serve as effective scaffolding for cells from the surrounding joint tissues and cartilage to populate and remodel into functional cartilage. Without being bound by a particular theory, proteins found in developing mammalian mesoderm (e.g., collagens and fibrillins) may enable recruitment of stem cells from surrounding tissue to heal the defect. Preclinical studies in animals have shown this technique can restore the articular surface and enable animals with arthritis to walk more normally within a few weeks of injection (see, the Examples herein). Moreover, it was found that treatment using methods provided herein were able to fill cartilage defects, reverse osteoarthritic gait changes, and slow the progression of PTOA observed by x-rays (in some cases stopping the progression of PTOA as noted on radiographs), even when the treatment occurred a significant time after the injury.

The methods provided herein require no surgery, and can be used to treat mammals (e.g., humans) with various types of arthritis. For example, mammals (e.g., humans) with idiopathic arthritis, inflammatory arthritis, rheumatoid arthritis, PTOA, or any other subtype of arthritis or cartilage damage can be treated using the compositions and methods provided herein.

In some cases, the methods and compositions provided herein can be used to fill a tissue defect. The tissue defect can be present in any tissue. For example, the tissue defect can be in a musculoskeletal connective tissue including, without limitation, articular cartilage, meniscus, bone, ligament, tendon, skin, and discs (e.g., the intervertebral discs of the spine and the temporomandibular joint disc). The defects can be full thickness defects or partial thickness defects, and can be visible defects or microscopic defects as found in tendinopathies. For intervertebral discs, the defects can involve the annulus fibrosis. The defects can be tissue defects in a joint (intra-articular) or outside of a joint. In some cases, a method provided herein can be used to fill tissue defects that result in pain or disability for the mammal being treated.

In some cases, the methods and compositions provided herein can be used to fill defects in articular cartilage. The articular cartilage defects can extend to the subchondral bone, can extend to the tidemark of the cartilage, or can be superficial to the tidemark. The defects can be partial thickness or full thickness defects, and can include fissures and/or shouldered or unshouldered lesions, and can range in size from about 0.1 mm to the entire articular surface (e.g., about 0.1 mm to about 0.3 mm, about 0.3 to about 0.5 mm, about 0.5 mm to about 1 mm, about 1 mm to about 2 mm, about 2 mm to about 4 mm, about 3 mm to about 5 mm, or more than 5 mm). The joint being treated can have one articular cartilage defect, or can have more than one articular cartilage defect (e.g., two, three, four, five, or more than five defects).

The joint being treated can be in the upper or lower extremity (also known as appendicular joints), or in the spine or other location in the body. Examples of appendicular joints include, without limitation, the knee joint, tibiotalar joint, subtalar joint, joints of the midfoot, metatarsophalangeal joints, metacarpal joints, metacarpal-phalangeal joints, other joints in the hand, wrist joint, elbow joint, and shoulder joint. Examples of joints in the spine include the facet joints. In some cases, the methods provided herein can be used to treat multiple partial thickness defects in the articular cartilage of the knee joint.

Thus, after mixing a powdered composition (either hydrated or unhydrated) with blood (or a fluid containing blood cells), the resulting mixture can be injected into a joint, a tissue defect, or an injury to be treated (e.g., an injury to a ligament, a tendon, bone, or cartilage, such as a meniscus, labrum, or disc). In some cases, a joint can be treated for degeneration without injury to the cartilage, ligament, tendon, meniscus, labrum, disc, or bone. Tissue defects that can be treated according to the methods provided herein can be present within a joint or outside a joint. In some cases, a tissue defect to be treated can be one that does not heal as quickly as desired by a patient or clinician, including, for example, cartilage injury or degeneration, bone fractures requiring internal fixation, open fractures, intra-articular fractures, rotator cuff tendon injuries, meniscus tears, labral tears, intervertebral disc herniations, degenerate temporomandibular disc injuries and injuries to the triangular fibrocartilage construct in the wrist.

In some cases, a hydrogel generated from a powdered mesodermal ECM and blood can be delivered via an injection rather via an incision. Such delivery can be advantageous, particularly in clinical situations such as, without limitation, treatment of delayed union of fractures (where an additional incision might further impair the local blood supply required to achieve healing), treatment of partial thickness rotator cuff tears (where the morbidity of the approach to those deep muscles may outweigh the potential benefits of repair), treatment of Achilles tendon rupture (where an additional incision could further impair the local blood supply and lead to increased wound healing problems), and treatment of tendinopathy without tissue rupture. In some cases, a hydrogel composition provided herein can be injected into a knee joint to treat a partial thickness cartilage defect.

In some cases, a hydrogel generated from a powdered mesodermal ECM and blood can be administered by direct injection from a syringe, or by any other appropriate means. For example, a hydrogel can be administered through an arthroscopic cannula or portal, or through an incision. In some cases, administration can be performed with the assistance of imaging techniques, such as ultrasound, magnetic resonance imaging, computed tomography, x-ray, fluoroscopy, or needle arthroscopy.

In some cases, tissue defects other than cartilage defects can be treated using the compositions and methods provided herein. For example, partial or complete tendon or ligament tears, meniscus injuries, and labral injuries can be treated by injection of a hydrogel composition provided herein, as can microscopic tissue injuries (e.g., tendinosis, tendinopathy, sprains, and strains of ligaments and tendons).

The methods provided herein can, in some cases, be used to treat those at risk for OA, including patients who have sustained previous injury or had a previous surgery. The methods provided herein can be used to treat mammals (e.g., humans) with any type of cartilage damage or arthritis. For example, the methods provided herein can be used to treat single cartilage defects, multiple cartilage defects, and cartilage damage due to rheumatoid arthritis, gout, inflammatory arthritis, psoriatic arthritis or infection. The methods also can be used to prevent or reduce the risk of developing future arthritis. For example, the methods provided herein can be used to prevent or reduce the likelihood of post-traumatic arthritis, where a specific joint injury may result in cartilage damage and arthritis years later. This can occur, for example, when a patient tears the anterior cruciate ligament of the knee, and despite having a surgical repair of that ligament, the patient still develops premature arthritis at an age far younger than would be seen for a patient who didn't have the injury. Such methods also can apply to patients with other injuries to the knee, such as meniscal injury, bone bruising, or cartilage injury. Further, the methods can be used in joints other than knee joints with a sustained injury. In some cases, a powder composition provided herein can be mixed with blood and administered by injection to a mammal (e.g., a human patient) having minor cartilage damage, to reduce the likelihood that the damage will progress to OA.

When a joint is surgically repaired, accidental damage may occur to the cartilage in the joint and lead to early arthritis. In some cases, the methods provided herein can include administering a composition containing a mesodermal ECM powder and blood at the end of a surgical procedure on a joint, either through an arthroscopic portal or by injection through closed skin or through an open incision, to reduce the likelihood that damage to the cartilage incurred during surgery will progress to arthritis. In some cases, the methods provided herein can include administering a mesodermal ECM powder/blood composition during a surgical procedure performed to treat another tissue (e.g., to help cartilage after ACL or meniscus surgery). The surgical procedure can be, for example, a partial meniscectomy, meniscus repair, ACL reconstruction, ACL repair, labral resection or repair of the hip or shoulder, treatment of osteochondritis dissecans in any joint, or any other surgical procedure performed on a joint.

In some cases, the methods provided herein can be used to supplement surgical repair of cartilage or other tissues. For example, when a microfracture procedure for an articular cartilage defect is performed as a primary treatment, a powdered mesodermal ECM and blood composition provided herein can be administered to the site after the microfracture procedure, to assist with healing. As another example, after a mosaicplasty using bone and cartilage plugs, a composition provided herein can be administered to assist with healing of the plugs, and to fill gaps between the surgically implanted plugs and the surrounding cartilage. In some cases, when a patch is placed to cover a defect in the cartilage, a composition provided herein can be injected under the patch, over the patch, or near the patch, to help accelerate healing. In some cases, when cells are injected into a cartilage defect, the cells can be first mixed with a composition provided herein, and then combination can be injected into the defect. In such cases, the powdered mesodermal ECM/blood composition can assist in localizing the cells to be delivered to the damaged tissue site. As another example, when a rotator cuff tendon is repaired with sutures and/or bone fixation, a composition provided herein be administered to the repaired tissue defect to improve healing. The administering can be done prior to the sutures being placed, or after placement of the sutures but before the sutures are tightened to re-approximate the tendon ends or tendon to bone, or after placement and tying of the sutures. In some cases, when a meniscus is repaired with sutures, a composition provided herein can be administered to the repaired tissue defect to improve healing. This can be done prior to the sutures being placed, or after placement of the sutures but before they are tightened to close the wound gap, or after the sutures are placed and tied. Similarly, a composition provided herein can be used to augment repair of shoulder labrum, hip labrum, ligaments including but not limited to the anterior cruciate ligament, posterior cruciate ligament, medial collateral ligament, lateral collateral ligament, talofibular ligaments, and ulnar collateral ligament, as well as tendons including but not limited to the Achilles tendon, flexor tendons of the hand, tendons attaching to the lateral epicondyle of the distal humerus, quadriceps or patellar tendons, and biceps tendon. For mechanically stable defects, including partial tears of ligaments or tendons, the methods provided herein can be used to treat these defects without the use of sutures.

### EXEMPLARY EMBODIMENTS

Embodiment 1 is a composition comprising a powdered extracellular matrix (ECM) component and a fluid, wherein the ECM component comprises collagen, and wherein the concentration of the powdered ECM component in the fluid is about 50 mg/mL to about 200 mg/mL.

Embodiment 2 is the composition of embodiment 1, wherein the concentration of the powdered ECM component in the fluid is about 100 to about 150 mg/mL.

Embodiment 3 is the composition of embodiment 1 or embodiment 2, wherein the fluid is blood.

Embodiment 4 is the composition of any one of embodiments 1 to 3, wherein the powdered ECM component has an average particle size of about 0.1 mm to about 1 mm.

Embodiment 5 is the composition of any one of embodiments 1 to 4, further comprising a growth factor, platelets, white blood cells, stem cells, a cross-linker, a neutralizing agent, or any combination thereof.

Embodiment 6 is the composition of any one of embodiments 1 to 5, wherein the composition further comprises calcium.

Embodiment 7 is the composition of any one of embodiments 1 to 6, wherein the composition is substantially free of one or more of nucleic acid, glycosaminoglycan (GAG), phospholipid, active pepsin, and active virus.

Embodiment 8 is a method for making a composition comprising a fluid and a powdered ECM component comprising collagen, wherein the method comprises:
providing a syringe containing the powdered ECM component, and
drawing an amount of the fluid into the syringe such that the concentration of the powdered ECM component in the fluid is about 50 mg/mL to about 200 mg/mL.

Embodiment 9 is the method of embodiment 8, comprising drawing an amount of the fluid into the syringe such that the concentration of the powdered ECM component in the fluid is about 100 to about 150 mg/mL.

Embodiment 10 is the method of embodiment 8 or embodiment 9, wherein the fluid is blood.

Embodiment 11 is the method of any one of embodiments 8 to 10, wherein the powdered ECM component has an average particle size of about 0.1 mm to about 1 mm.

Embodiment 12 is the method of any one of embodiments 8 to 11, wherein the ECM composition further comprises a growth factor, platelets, white blood cells, stem cells, a cross-linker, a neutralizing agent, or any combination thereof.

Embodiment 13 is the method of any one of embodiments 8 to 12, wherein the composition further comprises calcium.

Embodiment 14 is the method of any one of embodiments 8 to 13, wherein the composition is substantially free of one or more of nucleic acid, GAG, phospholipid, active pepsin, and active virus.

Embodiment 15 is a method for treating a mammal, wherein the method comprises administering to a joint of a mammal that has or is at risk for developing arthritis at the joint an effective amount of a composition comprising a powdered ECM component and a fluid, wherein the ECM component comprises collagen, and wherein the concentration of the powdered ECM component in the fluid is about 50 mg/mL to about 200 mg/mL.

Embodiment 16 is the method of embodiment 15, wherein the concentration of the powdered ECM component in the fluid is about 100 to about 150 mg/mL.

Embodiment 17 is the method of embodiment 15 or embodiment 16, wherein the fluid is blood.

Embodiment 18 is the method of any one of embodiments 15 to 17, wherein the powdered ECM component has an average particle size of about 0.1 mm to about 1 mm.

Embodiment 19 is the method of any one of embodiments 15 to 18, wherein the ECM composition further comprises a growth factor, platelets, white blood cells, stem cells, a cross-linker, a neutralizing agent, or any combination thereof.

Embodiment 20 is the method of any one of embodiments 15 to 19, wherein the composition further comprises calcium.

Embodiment 21 is the method of any one of embodiments 15 to 20, wherein the composition is substantially free of one or more of nucleic acid, GAG, phospholipid, active pepsin, and active virus.

Embodiment 22 is the method of any one of embodiments 15 to 21, wherein the mammal has an acute injury at the joint.

Embodiment 23 is the method of any one of embodiments 15 to 21, wherein the arthritis is osteoarthritis.

Embodiment 24 is the method of any one of embodiments 15 to 21, wherein the arthritis is post-traumatic arthritis.

Embodiment 25 is the method of embodiment 24, wherein the post-traumatic arthritis is associated with an intra-articular injury or arthroscopic surgery.

Embodiment 26 is the method of embodiment 25, wherein the intra-articular injury is selected from the group consisting of anterior cruciate ligament tear, anterior cruciate ligament rupture, meniscal injury, and cartilage injury.

Embodiment 27 is the method of any one of embodiments 15 to 26, wherein the mammal was surgically treated for a torn, fractured, strained, bruised, or ruptured intra-articular tissue at the joint at least one day prior to the administration of the composition.

Embodiment 28 is the method of any one of embodiments 15 to 26, wherein the joint is a joint of a hand, elbow, wrist, hip, knee, foot, shoulder, ankle, temporomandibular, or spine.

Embodiment 29 is the method of any one of embodiments 15 to 28, wherein the mammal has an injury associated with the development of arthritis.

Embodiment 30 is the method of any one of embodiments 15 to 29, wherein the administering comprises direct injection into the joint.

Embodiment 31 is the method of any one of embodiments 15 to 30, wherein the mammal is a human.

Embodiment 32 is a method for treating a mammal having an intra-articular tissue defect, the method comprising, after visualization of the defect with an arthroscope, administering to the defect an effective amount of a composition comprising a powdered ECM component and a fluid, wherein the ECM component comprises collagen, and wherein the concentration of the powdered ECM component in the fluid is about 50 mg/mL to about 200 mg/mL to the defect.

Embodiment 33 is the method of embodiment 32, wherein the concentration of the powdered ECM component in the fluid is about 100 to about 150 mg/mL.

Embodiment 34 is the method of embodiment 32 or embodiment 33, wherein the fluid is blood.

Embodiment 35 is the method of any one of embodiments 32 to 34, wherein the powdered ECM component has an average particle size of about 0.1 mm to about 1 mm.

Embodiment 36 is the method of any one of embodiments 32 to 35, wherein the ECM composition further comprises a growth factor, platelets, white blood cells, stem cells, a cross-linker, a neutralizing agent, or any combination thereof.

Embodiment 37 is the method of any one of embodiments 32 to 36, wherein the composition further comprises calcium.

Embodiment 38 is the method of any one of embodiments 32 to 37, wherein the composition is substantially free of one or more of nucleic acid, GAG, phospholipid, active pepsin, and active virus.

Embodiment 39 is the method of any one of embodiments 32 to 38, wherein the defect is an acute injury at the joint.

Embodiment 40 is the method of embodiment 39, wherein the defect is selected from the group consisting of anterior cruciate ligament tear, anterior cruciate ligament rupture, meniscal injury, and cartilage injury.

Embodiment 41 is the method of any one of embodiments 32 to 40, wherein defect is an injury associated with the development of arthritis.

Embodiment 42 is the method of any one of embodiments 32 to 41, wherein the administering comprises direct injection into the defect.

Embodiment 43 is the method of any one of embodiments 32 to 42, wherein the mammal is a human.

Embodiment 44 is a method for making a powdered composition comprising mesodermal extracellular matrix (ECM) proteins, the method comprising: decellularizing a tissue sample comprising tissue arising from mammalian mesoderm; treating the tissue sample, before or after decellularization, with a composition comprising peracetic acid; freeze-drying the decellularized tissue sample; and milling the freeze-dried tissue into a powder.

Embodiment 45 is the method of embodiment 44, wherein the composition comprising peracetic acid comprises about 0.1% peracetic acid.

Embodiment 46 is the method of embodiment 44 or embodiment 45, comprising treating the tissue sample, before or after decellularization, for about 5 to 30 minutes with the composition comprising peracetic acid.

Embodiment 47 is the method of any one of embodiments 44 to 46, wherein the composition comprising peracetic acid further comprises hydrogen peroxide.

Embodiment 48 is the method of embodiment 47, wherein the composition comprises about 1% hydrogen peroxide.

Embodiment 49 is the method of any one of embodiments 44 to 48, further comprising, prior to the freeze-drying, treating the decellularized tissue sample with an enzyme, thereby removing species-specific ends of collagen molecules.

Embodiment 50 is the method of any one of embodiments 44 to 49, further comprising treating the powder with supercritical carbon dioxide (scCO₂).

Embodiment 51 is the method of any one of embodiments 44 to 50, wherein the powder has an average particle size of about 0.1 mm to about 1 mm. Embodiment 52 is the method of any one of embodiments 44 to 51, wherein the composition further comprises a growth factor, platelets, white blood cells, stem cells, a cross-linker, a neutralizing agent, or any combination thereof.

Embodiment 53 is the method of any one of embodiments 44 to 52, wherein the composition further comprises calcium.

Embodiment 54 is the method of any one of embodiments 44 to 52, wherein the composition is substantially free of one or more of nucleic acid, GAG, phospholipid, active pepsin, and active virus.

Embodiment 55 is a method for making a powdered composition comprising mesodermal ECM proteins, the method comprising: decellularizing a tissue sample comprising tissue from mammalian mesoderm; freeze-drying the decellularized tissue sample; milling the freeze-dried tissue slurry into a powder; and treating the powder with scCO₂.

Embodiment 56 is the method of embodiment 55, further comprising, prior to the freeze-drying, treating the decellularized tissue sample with an enzyme, thereby removing species-specific ends of collagen molecules.

Embodiment 57 is the method of embodiment 55 or embodiment 56, further comprising treating the tissue sample, before or after decellularization, with a composition comprising peracetic acid.

Embodiment 58 is the method of embodiment 57, wherein the composition comprising peracetic acid comprises about 0.1% peracetic acid.

Embodiment 59 is the method of embodiment 57 or embodiment 58, comprising treating the tissue sample, before or after decellularization, for about 5 to 30 minutes with the composition comprising peracetic acid.

Embodiment 60 is the method of any one of embodiments 57 to 59, wherein the composition comprising peracetic acid further comprises hydrogen peroxide.

Embodiment 61 is the method of embodiment 60, wherein the composition comprises about 1% hydrogen peroxide.

Embodiment 62 is the method of any one of embodiments 55 to 61, wherein the powder has an average particle size of about 0.1 mm to about 1 mm.

Embodiment 63 is the method of any one of embodiments 55 to 62, wherein the composition further comprises a growth factor, platelets, white blood cells, stem cells, a cross-linker, a neutralizing agent, or any combination thereof.

Embodiment 64 is the method of any one of embodiments 55 to 63, wherein the composition further comprises calcium.

Embodiment 65 is the method of any one of embodiments 55 to 64, wherein the composition is substantially free of one or more of nucleic acid, GAG, phospholipid, active pepsin, and active virus.

Embodiment 66 is a method for making a composition comprising a blood and a powdered ECM component comprising collagen, wherein the method comprises: providing a syringe containing the powdered ECM component: contacting a sample of blood with an anticoagulant; drawing an amount of the blood into the syringe containing the powdered ECM component such that the concentration of the powdered ECM component in the blood is about 50 mg/mL to about 200 mg/mL; and adding a calcium chloride solution to the syringe, thereby deactivating the anticoagulant.

Embodiment 67 is the method of embodiment 66, comprising drawing an amount of the blood into the syringe such that the concentration of the powdered ECM component in the blood is about 100 to about 150 mg/mL.

Embodiment 68 is the method of embodiment 66 or embodiment 67, wherein the powdered ECM component has an average particle size of about 0.1 mm to about 1 mm.

Embodiment 69 is the method of any one of embodiments 66 to 68, wherein the ECM composition further comprises a growth factor, platelets, white blood cells, stem cells, a cross-linker, a neutralizing agent, or any combination thereof.

Embodiment 70 is the method of any one of embodiments 66 to 69, wherein the composition is substantially free of one or more of nucleic acid, GAG, phospholipid, active pepsin, and active virus.

Embodiment 71 is the method of any one of embodiments 66 to 70, wherein the calcium chloride solution has a concentration of about 35 mM to about 45 mM.

Embodiment 72 is the method of any one of embodiments 66 to 71, comprising adding the calcium chloride solution to the syringe to obtain a mixture comprising a 1:9 ratio of calcium chloride solution to blood.

The invention will be further described in the following examples, which do not limit the scope of the invention described in the claims.

### EXAMPLES

### Example 1—Slowing Post-Traumatic Osteoarthritis In Vivo

Sixty Lewis rats were randomized to one of three groups (20 animals per group) and had unilateral surgery as specified below for each group:
(1) capsulotomy but no ACL injury/treatment (sham group),
(2) ACL transection and injection with PBS (placebo group),
(3) ACL transection and injection with a composition containing multiple mesodermal proteins including collagen and fibrillin (treatment group).

At six weeks after surgery, the knees were evaluated for the presence of PTOA as evidenced by changes on radiographs (X-rays) and on microscopic evaluation of the cartilage (histology).

A mesodermal protein composition was aseptically manufactured from decellularized bovine elastic tissues. Bovine elastic tissue (Maverick BioSciences, New Zealand) was incubated in an antibiotic solution to inactivate any contaminating infectious particles acquired during harvest, and the tissue was then decellularized using Triton X-102. The tissue was rinsed and treated with pepsin digestion and solubilization in hydrochloric acid. The resultant slurry of mesodermal proteins was lyophilized and then rehydrated to concentrate the collagen to at least 45 mg/g of slurry. The concentrated slurry was then neutralized using NaOH and a HEPES (4-(2-hydroxyethyl)-1-piperazineethanesulfonic acid) buffer, and aqueous CaCl₂ was used to bring the osmolarity of the slurry to 295 mOsm. The slurry was then lyophilized and was milled into a powder. Twenty (20) mg doses of the powder composition were loaded into individual 1 mL syringes. The loaded syringes were stored at room temperature and protected from light.

For the animal surgeries, an anteromedial approach was used to expose the ACL, which was transected sharply under direct vision, and then the capsule was closed. For the sham group, only the exposure of the ACL was performed. For the placebo group, after ACL transection and closure of the capsule, a saline injection was performed under ultrasound guidance. For the treatment group, 20 mg of the protein powder composition containing collagen and fibrillin was initially rehydrated with 400 μL water and mixed between two syringes to create a hydrated composition. 200 μL of the hydrated composition was then mixed with 200 μL of autologous blood to form a homogenous mixture, and 100 μL of the two-part mixture was then injected into the synovial fluid of the knee using a 25-gauge ⅝″ needle. The cartilage was not visualized during the injection and the injection was not placed directly onto the cartilage, but rather into the fluid within the joint cavity.

Mass spectroscopy confirmed the presence of multiple mesodermal proteins in the mesodermal protein powder composition used in the treatment group, including Type I collagen, Type III collagen, Fibrillin-1, Type VI collagen, Types II collagen, Type V collagen, Type VI collagen, fibrillin-2 precursor, and laminin. The collagen concentration in the composition was about 500,000 μg/g powder, the DNA content was about 40,000 ng/g powder, and the residual pepsin content was below 122 mg/g powder (the lower limit of the assay). Sterility of the mesodermal protein powder composition was confirmed using bioburden testing. Gelation of the aseptically processed mesodermal protein composition occurred at room temperature and at 37° C., with no blurring of the cut surface when the samples were unmolded and cut with a straight blade.

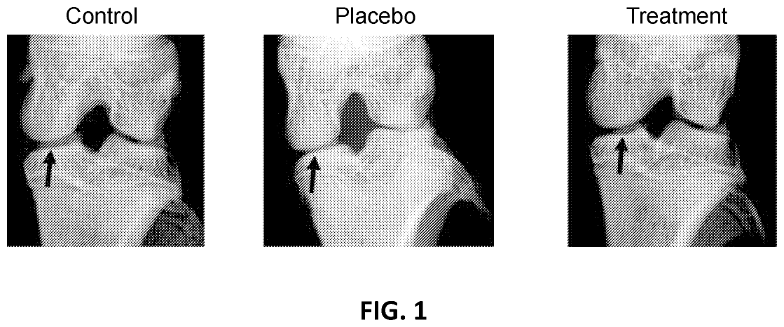

At six weeks after surgery and injection, radiographs of the knees were taken. Exemplary images are shown in FIG. 1. In the sham group, where no ACL transection was performed (left panel), there was no loss of joint space, normal subchondral bone appearance, and no osteophytes visible. In the knees treated with ACL transection followed by an injection of PBS (placebo group, center panel), by six weeks there was significant joint space narrowing accompanied by subchondral bone sclerosis and osteophyte formation. In contrast, in the knees treated with ACL transection followed by an injection of aseptically processed mesodermal protein composition mixed with autologous blood, at six weeks after surgery the knees had maintained their joint space (black arrow), and there was no thickening of the subchondral bone or no osteophyte formation. This demonstrated that in the rat model, ACL transection treated with a placebo injection led to radiographic signs of PTOA at six weeks after surgery. Injection of the two-part mixture of mesodermal protein composition and blood at the time of trauma (ACL transection) prevented the development of post-traumatic osteoarthritis.

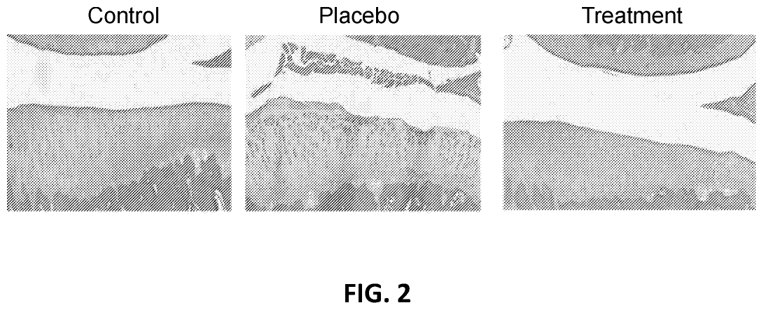

FIG. 2 includes images showing the histology of the medial tibial plateau for a representative knee that did not have any surgery (left panel), a representative knee that had ACL transection and a placebo injection of saline six weeks prior (center panel), and a knee that had ACL transection and an injection of the two-part mixture at the time of the ACL transection surgery six weeks prior (right panel). The knees that had no surgery had normal appearing cartilage, while the knees that had ACL transection with a placebo injection had an irregular cartilage surface and loss of matrix (center panel). In contrast, knees that had an ACL transection and were treated with the mixture of mesodermal proteins and blood had normal appearing cartilage. Thus, injection of a mesodermal protein composition after ACL injury prevented the onset of PTOA.

Quantitative histology using the Mankin structural score revealed severe arthritic changes (defined as a score of 7 or 8) in 11% of the knees that did not have an ACL transection (SHAM group), 21% of the knees treated with PBS, and 5% of the knees that had an ACL transection followed by an injection of the mesodermal protein composition mixed with blood.

### Example 2—Treating OA without a Prior Injury

The Dunkin Hartley guinea pig spontaneously develops OA of the knee between six and nine months of life, and is a widely recognized and utilized model of naturally occurring OA. While humans develop spontaneous OA typically over decades, the Dunkin Hartley animals develop it within several months.

In these experiments, twenty 6-month-old Dunkin Hartley Guinea Pigs were divided between two experimental groups: one group (n=5) had no injections performed (control group). The remaining fifteen animals were treated with a single injection in each knee of a mesodermal protein composition and blood at week 0 (treatment group). Five animals were euthanized at 1, 2, and 4 weeks after the injection, and their knees were evaluated histologically. A mesodermal protein powder was made by decontaminating bovine elastic tissue with a solution containing hydrogen peroxide, decellularizing the tissue, and rinsing and lyophilizing the tissue. After lyophilization, the tissue was digested with pepsin in an acid solution, and the resulting slurry was neutralized, lyophilized, and milled into a powder before being loaded into 10 mL syringes in doses of 125 mg each, packaging the syringes in sealed packaging and terminally sterilizing the powder using at least 20 kGy of electron beam radiation.

Mass spectroscopy confirmed the presence of multiple mesodermal proteins in the mesodermal protein powder used in the treatment group, including Type I collagen, Type III collagen, Fibrillin-1, Type VI collagen. Types II collagen, Type V collagen, Type VI collagen, fibrillin-2 precursor, and laminin and other extracellular matrix proteins. The collagen concentration in the composition was about 500,000 μg/g powder, the DNA content was about 40,000 ng/g powder, and the residual pepsin content was below 122 mg/g powder (the lower limit of the assay). The sterility of the mesodermal protein powder after sterilization was confirmed. The ability of the composition to self-assemble was assessed by mixing a 125 mg sample of the powder with 0.5 mL water. A sample of the mixture was checked for the ability of the contained proteins to self-assemble and form a gel by allowing the sample to sit in a cylindrical tube at 32° C. (the temperature of the knee joint). This resulted in transformation of the liquid composition into a solid gel, indicative of successful self-assembly, within 60 minutes. The gel was easily removed from the tube and maintained a cylindrical shape after placement on a flat surface. The stability of the gel was tested by cutting the gel cylinder with a straight blade and evaluating for shape loss. There was with no blurring of the cut surface or loss of column integrity when the test samples were cut with a straight blade.

The two-part mesodermal protein composition/blood mixture was injected by adding 0.5 mL water to the prepared syringe containing 125 mg of the lyophilized extracellular matrix proteins to form a hydrated composition. 100 μl of the hydrated composition was placed in a 1 mL syringe. Blood was drawn autologously from each animal into a syringe containing an anticoagulant. Just prior to mixing with the hydrated composition, calcium chloride was added to the blood to de-activate the anticoagulant. 200 μl of autologous blood was mixed with the 100 μl hydrated composition to form a homogenous mixture. After the hydrated composition was mixed with the autologous blood, it was injected into the synovial fluid of the knee without visualization of the cartilage. Histology of the medial tibial plateau was performed on knees at 1, 2, and 4 weeks after injection. Gait assessment (base of support) was performed at baseline (time 0) and then at 1, 2, and 4 weeks post-injection in for the control and treated groups.

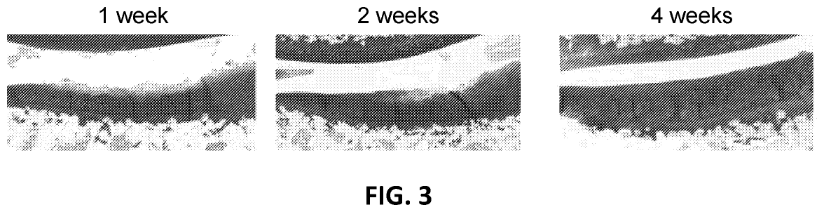

Histologic analysis of the articular cartilage revealed that at one week after injection, the area of the medial tibial plateau where the superficial cartilage had been lost due to spontaneous arthritis was filled in with the mesodermal protein composition/blood mixture material. Toluidine blue stains glycosaminoglycans, and as shown in FIG. 3, the area of mesodermal protein composition/blood mixture (which has significantly less glycosaminoglycan than native cartilage) appeared as the light blue area on the surface of the damaged cartilage. No cells were visible in the mesodermal protein composition/blood mixture material at the one-week time point (left panel). By two weeks after injection, some of the lighter blue mesodermal protein composition/blood mixture material was replaced by articular cartilage (greater glycosaminoglycan content and now containing chondrocytes), although there were still some areas with the acellular mesodermal protein composition/blood mixture with low glycosaminoglycan (center panel, lighter area). By four weeks after the mesodermal protein composition/blood mixture injection, the entire scaffold was populated by chondrocytes, and the superficial zone of cartilage was restored with a high glycosaminoglycan material consistent with articular cartilage and containing chondrocytes (right panel). The lamina splendens appeared intact as well.

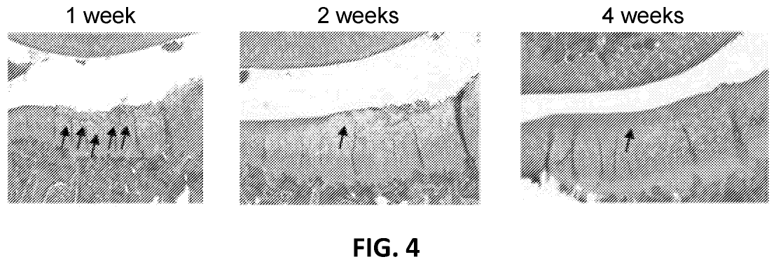

FIG. 4 shows the histology of the medial tibial plateau of the treated guinea pigs at 1 week (left panel), 2 weeks (center panel), and 4 weeks (right panel) after mesodermal protein composition/blood mixture injection. Masson's Trichome stain was used, such that collagen and bone appeared blue, cell cytoplasm appeared light red/pink, and nuclei appeared black. In the 1-week animals, the lost superficial zones of cartilage had been filled by the acellular mesodermal protein composition/blood mixture (lighter material and black arrows, left panel). At two weeks, the surrounding chondrocytes had started to reconstitute the superficial zones of cartilage where the mesodermal protein composition/blood mixture had previously been (black arrow, center panel). By four weeks after injection, the mesodermal protein composition/blood mixture had been completely replaced by chondrocytes and a high GAG matrix and the superficial zones of cartilage (including the lamina splendens) had been reconstituted by the native chondrocytes (right panel).

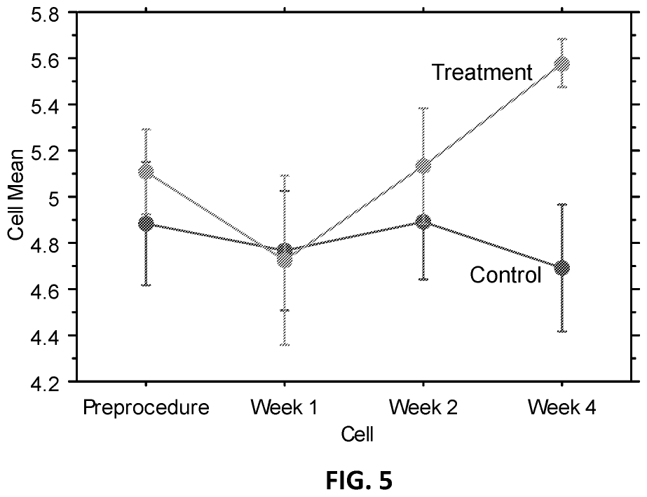

Gait analysis was performed to assess changes in the Base of Support (BOS) in animals who were untreated vs. those that received treatment with the mesodermal protein composition/blood mixture. The treated animals had higher mean BOS than the controls at 2 and 4 weeks after injection (FIG. 5), demonstrating a prolonged improvement in osteoarthritis after injection.

Taken together, these studies demonstrate that in a spontaneous model of arthritis, injection of a mesodermal protein composition/blood mixture containing fibrillin and collagens was able to fill in arthritic cartilage defects and provided a protected space for the surrounding native cells to produce new articular cartilage, effectively resurfacing the joint and treating the arthritis. This restoration of the cartilage resulted in an improved gait of the animals, similar to that described elsewhere for anti-inflammatory administration in this same model (Santangelo et al., Arthritis, 2014(2):503519, doi:10.1155/2014/503519). However, the mesodermal protein composition/blood mixture effects lasted for four weeks, whereas the anti-inflammatory treatment lasted less than 24 hours. Since the guinea pig life span is about 4 to 8 years, one guinea pig year is roughly equivalent to a decade of human life, so four weeks of improvement in the guinea pig model may translate into six months or longer of relief for human patients. These studies demonstrated that use of a mesodermal protein composition containing collagen and fibrillin was able to fill osteoarthritic defects and allow for cartilage healing, resulting in a reversal of the cartilage and gait changes seen in osteoarthritis, and effectively treating the spontaneous osteoarthritis.

### Example 3—Powder Vs. a Larger Single Scaffold

Studies were conducted to evaluate the retention of absorbed liquid after centrifugation for a powder formulation provided herein, as compared with a porous bridge-enhanced anterior cruciate ligament repair (BEAR) scaffold. The mesodermal protein powder was made by decontaminating bovine elastic tissue with a solution containing hydrogen peroxide, decellularizing the tissue, and rinsing and lyophilizing the tissue. After lyophilization, the tissue was digested with pepsin in an acid solution, and the resulting slurry was neutralized, lyophilized, and milled into a powder with a particle size between 0.1 and 0.6 mm. For this study 250 mg of powder was mixed with 3 mL of phosphate buffered saline and centrifuged. The porous scaffolds were soaked in phosphate buffered saline prior to centrifugation. These studies demonstrated that over 99% of the absorbed liquid remained in the hydrogel generated from the powder formulation, even when centrifuged at high speeds. This was not seen with porous scaffolds, where over 20% of the liquid was lost during centrifugation. Results are shown in TABLES 1A and 1B.

TABLE 1A
Retention of absorbed liquid in powder-containing hydrogel

| Sample | J7 | J8 | J9 | J10 | J11 | J12 | Avg. |
|---|---|---|---|---|---|---|---|
| Empty Tube (g) | 975 | 982 | 998 | 977 | 982 | 974 | |
| Tube + Plug (g) | 1207 | 1198 | 1212 | 1228 | 1230 | 1219 | |
| Initial Plug (g) | 232 | 216 | 214 | 251 | 248 | 245 | |
| Tube + Plug post | 1207 | 1197 | 1212 | 1228 | 1230 | 1219 | |
| centrifugation (g) | | | | | | | |
| Plug Post | 232 | 215 | 214 | 251 | 248 | 245 | |
| Centrifugation (g) | | | | | | | |
| Plug Post | 100.0% | 99.5% | 100.0% | 100.0% | 100.0% | 100.0% | 99.9% |
| Centrifuge/Initial | | | | | | | |
| (%) | | | | | | | |

In TABLE 1A the average value (99.9%) represents the retained weight of the gelled composite (the “plug”) following centrifugation, as compared to the weight of the initial gelled composite. These data show that the centrifugal force provided by sustained centrifugation (20,000 rcf for 10 minutes) did not result in the separation of fluid from the powder. In contrast, as shown in TABLE 1B, when sections of a scaffold (BEAR) were soaked in fluid to the point of saturation and then centrifuged, less than 80% of the wet weight remained, due to fluid separation following exposure to centrifugal forces (20,000 rcf for 10 minutes).

TABLE 1B
Retention of absorbed liquid in a fluid-soaked scaffold

| Sample | B7 | B8 | B9 | B10 | B11 | B12 | Avg. |
|---|---|---|---|---|---|---|---|
| Empty Tube (g) | 974 | 994 | 991 | 997 | 992 | 985 | |
| Tube + Plug (g) | 1176 | 1133 | 1164 | 1153 | 1150 | 1134 | |
| Initial Plug (g) | 202 | 139 | 173 | 156 | 158 | 149 | |
| Tube + Plug post | 1126 | 1100 | 1114 | 1131 | 1111 | 1115 | |
| centrifugation (g) | | | | | | | |
| Plug Post | 152 | 106 | 123 | 134 | 119 | 130 | |
| Centrifugation (g) | | | | | | | |
| Plug Post | 75.2% | 76.3% | 71.1% | 85.9% | 75.3% | 87.2% | 78.5% |
| Centrifuge/Initial (%) | | | | | | | |

### Example 4—Effects of Particle Size (Unsterilized Powder)

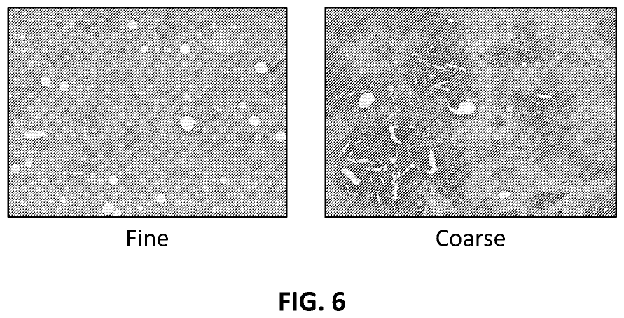

A series of studies were conducted to determine if a finer particle size would result in changes in the gelation characteristics or mechanical properties of a hydrogel with aseptic (not e-beam sterilized) powder. Fine particles having a diameter less than 0.3 mm were compared with coarse particles having a diameter between 0.3 mm and 0.6 mm. 400 mg of each powder was mixed with 3 mL of PRP. Both the fine and coarse particles exhibited excellent gelation, with scores of 6/6, and had similar mechanical properties. The gelation was a sum score of shape (score 0=no shape retention to score 3=shape contours remain sharp) and cut surface (score 0=gel too soft to cut to score 3=cut surfaces stay sharp). The difference between the particles was more apparent upon histology evaluation (FIG. 6), as the fine particles were more uniformly distributed within the diluent (left panel) than the coarse particles (right panel). In addition, the use of a finer particle size led to more rapid dissolution by collagenase. TABLE 2 includes data from a study in which 9 mm plugs generated from 200 or 400 mg coarse or fine powder in PRP were digested in 200 u/mL collagenase at 32° C. for 2 hours.

TABLE 2
Collagenase degradation

| | 200 mg | 200 mg | 400 mg | 400 mg | |
|---|---|---|---|---|---|
| | fine | coarse | fine | coarse | PRP^{§} |
| N | 4 | 4 | 4 | 4 | 2 |
| Minimum | 0.8391* | 0.8731 | 0.7997 | 0.8415 | 0.7943 |
| Maximum | 0.8815 | 0.9293 | 0.8707 | 0.9277 | 0.8592 |
| Range | 0.04239 | 0.05626 | 0.07101 | 0.08613 | 0.06492 |
| Mean | 0.8592 | 0.8922 | 0.8367 | 0.8967 | 0.8268 |
| Std. Dev. | 0.01744 | 0.02537 | 0.03356 | 0.03911 | 0.04590 |
| SEM | 0.008721 | 0.01269 | 0.01678 | 0.01956 | 0.03246 |

*% weight remaining, normalized to the % weight remaining in control samples digested in phosphate buffered saline without collagenase.
^{§}PRP, platelet-rich plasma.

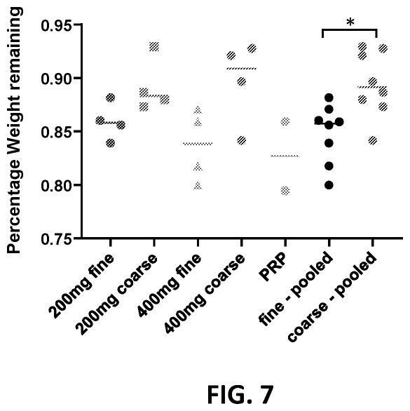

Normalized to the percentage of weight remaining in the control samples, the 200 mg fine powder samples retained 86% of their weight, the 200 mg coarse powder samples retained 89% of their weight, the 400 mg fine powder samples retained 84% of their weight, and the 400 mg coarse powder samples retained 90% of their weight. The PRP samples retained the least weight at 83%. Statistically significant differences were not found when individual groups were compared using a one-way ANOVA. After pooling fine and coarse groups, however, there was a statistically significant difference (p<0.01), where the average percentage of weight loss was larger in the fine groups than the coarse groups. Values are plotted in FIG. 7.

The experiments discussed above suggested that for applications in which collagenase resistance is desired, a particle size between 0.3 mm and 0.6 mm is likely to be more useful, while for applications in which faster resorption and a more uniform product is required, a particle size less than 0.3 mm in diameter is likely to be more useful. In addition, it is noted that the finer powder took up a smaller volume when loading the syringe, which increased the upper limit of the powder/fluid ratio.

### Example 5—Effects of Powder Concentration (Unsterilized Powder)

### Gel Shape Testing

Gel shape testing studies were conducted to evaluate the effect of powder concentration on shape retention. Gels were prepared in 5 mL syringes, using 200 mg or 400 mg powder in 3 mL of PRP, with PRP as a control. Nine (9) mm plugs from each sample were evaluated for shape retention and cutting ability after a 2 hour incubation in a 32° C. water bath. The PRP samples did not retain their original cylindrical shape during the incubation, but rather contracted to a cylinder with a diameter of about 3 mm. These samples were therefore excluded from subsequent gel shape and compression analysis, although they were included in the collagenase resistance testing.

The gel shape of the 200 mg and 400 mg powder per 3 mL of PRP samples was tested for the first 9 mm plug from each of four syringes for each amount of powder, and scored in accordance with the categories shown in TABLE 3. Results of the testing are set forth in TABLE 4.

The 400 mg samples resulted in a significantly stronger gel that held its cylindrical shape, completely retained its shape after cutting, and presented cut surfaces with well-defined edges. The 200 mg samples were clearly softer and showed slight sagging of the round cylindrical shape. After cutting, the cut edges were less sharp than the edges of the cut 400 mg samples.

TABLE 3
Gel shape/cut scoring criteria

| Score | Shape | Cut |
|---|---|---|
| 0 | No observable shape retention; | Cut not possible. |
| | contents dissociate and expand into the | |
| | petri dish. No self-assembly obvious. | |
| 1 | Minimal shape retention. Loss of | Able to cut, slightly more viscous |
| | cylindrical shape. Contents resemble | but cut sections collapse; |
| | initial viscous slurry. | consistency resembles toothpaste. |
| 2 | Moderate shape retention. Cylindrical | Able to cut, slight deformation of |
| | shape maintained with slight rounding | cut slices with rounded edges. |
| | of edges. | Resembles pudding. |
| 3 | Significant shape retention. Edges of | Able to cut, complete retention of |
| | the plug remain sharp and well | shape in cut slices with well- |
| | defined. | defined edges. Resembles jello. |

TABLE 4
Gel shape scores

| Particle Size | Shape Score | Cut Score | Total Score | Lot AVG |
|---|---|---|---|---|
| 200 | 2 | 2 | 4 | 4 |
| 200 | 2 | 2 | 4 | |
| 200 | 2 | 2 | 4 | |
| 200 | 2 | 2 | 4 | |
| 400 | 3 | 3 | 6 | 6 |
| 400 | 3 | 3 | 6 | |
| 400 | 3 | 3 | 6 | |
| 400 | 3 | 3 | 6 | |

### Gel Compression Testing

Studies were conducted to test the effect of powder concentration on resistance to gel compression. The gel compression testing of 200 mg and 400 mg powder in 3 mL of PRP were tested on the second 9 mm plug from each of the 4 syringes, and scored in accordance with the criteria shown in TABLE 5. Measurements of the diameter and compression scores are shown in TABLE 6.

TABLE 5
Gel compression scoring criteria

| Score | Criteria |
|---|---|
| 0 | No cylindrical shape upon expulsion from syringe, |
| | unable to perform compression test. |
| 1 | Plug Diameter* >200% initial diameter after 15 |
| | minutes under 10 g weight. |
| 2 | Plug Diameter* 150-200% initial diameter after |
| | 15 minutes under 10 g weight. |
| 3 | Plug Diameter* <150% initial diameter after 15 |
| | minutes under 10 g weight. |

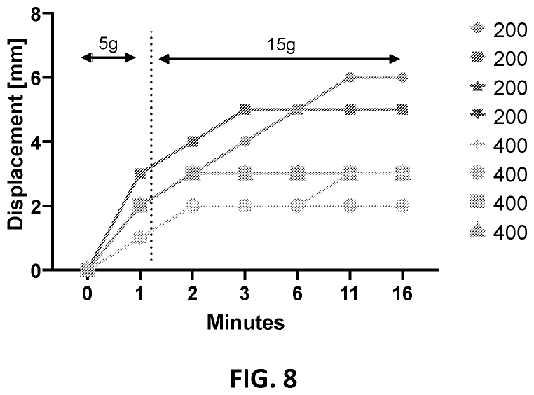

Compression testing revealed a significantly higher resistance to deformation for the 400 mg samples as compared to the 200 mg samples. The diameter of the 200 mg samples increased under 0.05 N load from 11.5 mm to about 15 mm within 1 minute, and to about 17 mm under 0.15 N, reaching a plateau after 2 to 5 minutes. The deformation was plastic. The diameter of the 400 mg samples increased diameter to about 13.5 mm under 0.05 N load, while leveling out at 15 mm under 0.15 N load. The deformation was also plastic. A displacement curve for the samples is shown in FIG. 8.

TABLE 6
Plug diameters and compression scores

| | | Max. | | | | | | |
|---|---|---|---|---|---|---|---|---|
| | Max. | Diam. | | | | | | |
| | Diam. | 1 min. | | | | | | |
| | 1 min. | under | Max. | Max. | Max. | Max. | | |
| Particle | under | 10 g | Diam. | Diam. | Diam. | Diam. | Compression | Lot |
| size | dish | weight | 2 min. | 5 min. | 10 min. | 15 min. | Score | AVG |
| 200 | 14 | 15 | 16 | 17 | 18 | 18 | 2 | 2 |
| 200 | 15 | 16 | 17 | 17 | 17 | 17 | 2 | |
| 200 | 15 | 16 | 17 | 17 | 17 | 17 | 2 | |
| 200 | 15 | 16 | 17 | 17 | 17 | 17 | 2 | |
| 400 | 13 | 14 | 14 | 14 | 15 | 15 | 3 | 3 |
| 400 | 13 | 14 | 14 | 14 | 14 | 14 | 3 | |
| 400 | 14 | 15 | 15 | 15 | 15 | 15 | 3 | |
| 400 | 14 | 15 | 15 | 15 | 15 | 15 | 3 | |

### Gel Collagenase Testing

To assess the effect of concentration on resistance to collagenase digestion, two quarters of the first 9 mm disc from each syringe were digested in 1 mL of 200 u/mL collagenase solution at 32° C. for 2 hours, and then centrifuged at 20,000×g for 10 minutes before decanting the supernatant. Weights of the quarters were recorded before and after digestion, and were compared to controls that had been incubated in buffer without collagenase. The percentage weight remaining in the control samples was averaged, and the remaining weight percentage of the collagenase-treated samples was normalized to the average control percentages. TABLE 7 shows the statistics of the findings in each group.

TABLE 7
Statistics for gel collagenase testing

| | 200 mg | 400 mg | PRP |
|---|---|---|---|
| Number of values | 8 | 8 | 2 |
| Minimum | 0.5610 | 0.7396 | 0.7943 |
| Maximum | 0.7591 | 0.8321 | 0.8592 |
| Range | 0.1982 | 0.09253 | 0.06492 |
| Mean | 0.6663 | 0.7890 | 0.8268 |
| Std. Deviation | 0.06171 | 0.03205 | 0.04590 |
| Std. Error of Mean | 0.02182 | 0.01133 | 0.03246 |

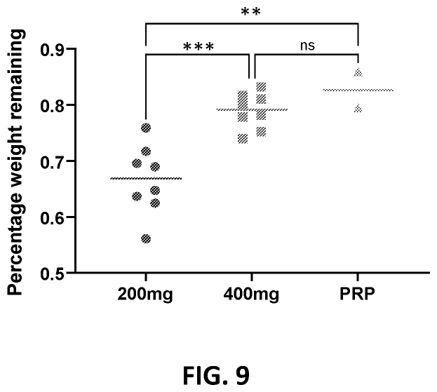

Normalized to the percentage of weight remaining in the control samples, the 200 mg samples retained 66% of their weight, while the 400 mg samples retained 79%. The PRP samples retained the most weight, at 83%. Statistically significant differences were observed between the 200 mg and the 400 mg groups (p<0.001), as well as between the 200 mg and the PRP groups (p<0.01). Values are plotted in FIG. 9.

### Example 6—Effects of Vacuum Mixing (Unsterilized Powder)

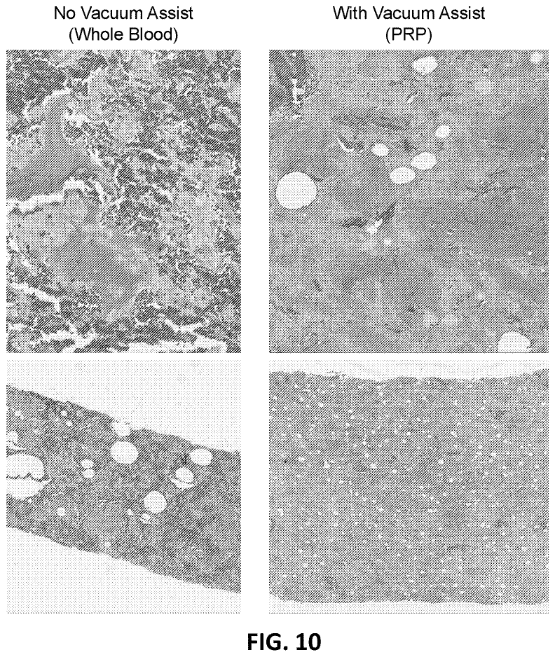

Studies were conducted to assess the effect of vacuum-assist mixing on various characteristics of the hydrogels. These studies demonstrated, for example, that vacuum-assist mixing improved the uniformity of hydration. A coarse powder was mixed with blood with and without vacuum assist. Images of the resulting mixtures are shown in FIG. 10. The large, smooth, lighter areas in the left panels are unmixed particles, and the darker, granular areas are red blood cells. In the right panels, vacuum mixing was used to hydrate the powder with PRP devoid of red blood cells. Fewer areas of pure blood were observed, and instead there appeared to be a more uniform pattern of mixing.

### Example 7—Data for Sterilized Powder Material

### Composition

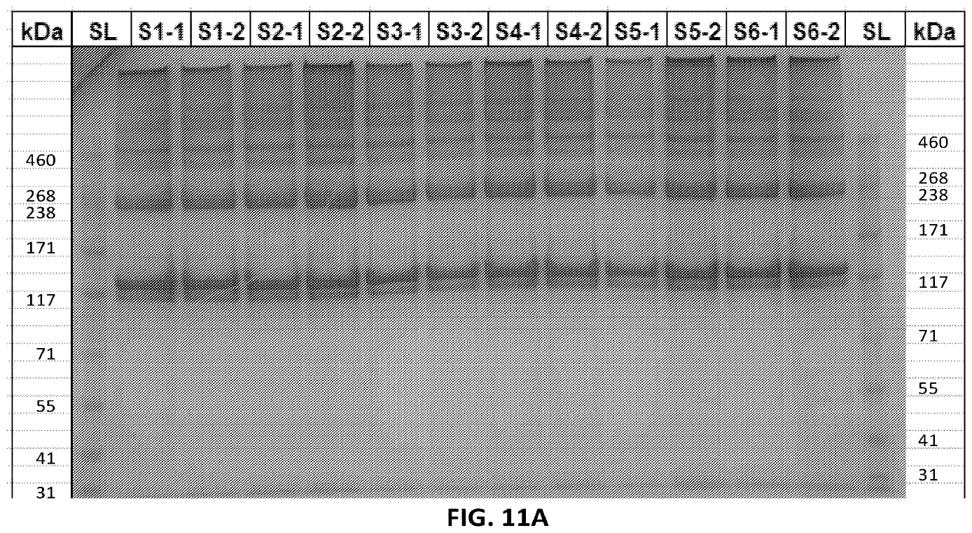

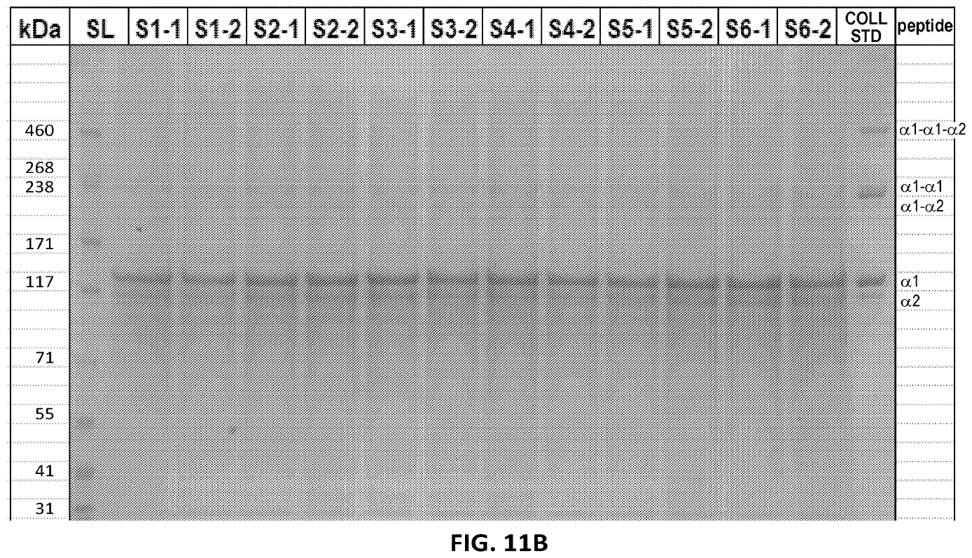

The process of terminal sterilization typically is harsh and can alter the protein structure of underlying ECM derived materials, resulting in changes to composition and mechanical characteristics. A range of 20-25 kGy electron beam sterilization can be used to achieve a sterility assurance level (SAL) of 10⁻⁶ when the bioburden level of aseptically manufactured material is below 45 colony forming units (CFU). Irradiation- (e-beam-) induced changes to the protein structure can be visualized by SDS-PAGE. For example, FIG. 11A is an image showing SDS-PAGE evaluation of aseptically manufactured powder prior to sterilization. FIG. 11B shows SDS-PAGE using e-beam sterilized powder at the same sample dilution used for FIG. 11A. The far-right lane in FIG. 11B (“COLL STD”) included a collagen standard dilution from the Sircol Collagen Assay to show the alpha monomers, beta dimers, and gamma trimer of collagen as a reference. In FIG. 11B, smearing of the bands occurred due to chain scissions in collagen molecules, which were caused by the high energy irradiation of e-beam processing.

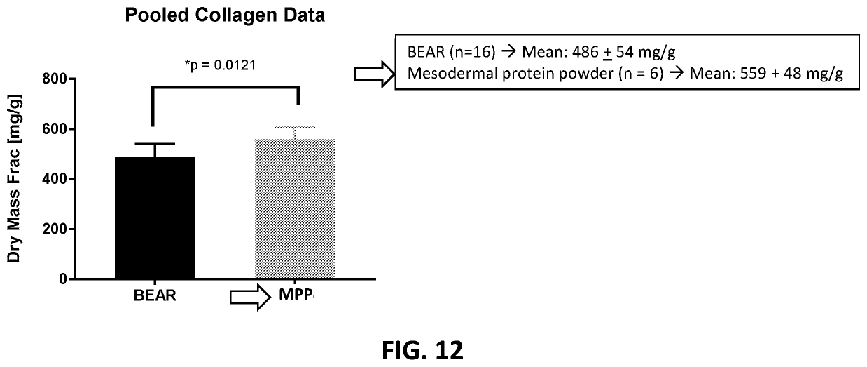

Despite changes to the collagen profile indicated by the SDS-PAGE studies, a quantitative Sircol Soluble Collagen Assay, which uses a dye that binds to helical portions of soluble collagens, was able to detect and measure collagen levels above 500 mg/g (>50% dry weight), indicating that the collagen molecules were not broken down beyond recognition. Unlike BEAR scaffolds that are brought to iso-osmolarity using calcium chloride and buffered with HEPES before lyophilization of the slurry into the scaffold, the powder provided herein was only brought to a pH above 8.5 to denature pepsin (the enzyme used to form the digest following tissue decellularization) and was then returned to a neutral pH of 7-7.4 using dilute sodium hydroxide and hydrochloric acid (referred to herein as “pH 8.5→7”). Thus, the salt content of the powder was much lower than that of the BEAR scaffolds, and the protein and collagen mass fraction (dry wt/wt) was higher in turn. This is the main reason for the higher collagen content in the powder provided herein (levels above 500 mg/g, whereas the BEAR scaffold showed average levels below 500 mg/g). The lot averages of collagen dry weight mass fractions for BEAR scaffolds and powders provided herein were pooled and statistically analyzed using an unpaired parametric t-test with Welch's correction. On average, the powder had a collagen content that was 73 mg/g higher than the BEAR scaffolds, a difference that was statistically significant (FIG. 12).

### Mechanical Characteristics

A study of the mechanical characteristics of e-beam sterilized powder demonstrated that particle size and pH adjustment between tissue digestion and final freeze drying did not significantly impact the mechanical integrity of reconstituted gels (powder mixed with buffered saline and incubated at knee joint temperature), but the powder to fluid ratio did impact mechanical integrity.

### Variables/Groups

1. Neutralization method
   a. pH8.5→7
   b. pH 6, lyophilized the same day
   c. pH 6, allowed to sit for 3 days before lyophilization
2. Powder size
   a. FINE=through size 50 mesh (<0.3 mm)
   b. COARSE=through size 30 mesh, not through size 50 mesh (0.3 mm>powder>0.6 mm)
3. Powder/phosphate buffered saline (PBS) ratio
   a. 200 mg powder/3 mL PBS
   b. 400 mg powder/3 mL PBS

### Test Measures—Gelation

1. Shape cut scoring (TABLE 8)
2. Compression testing under load across 15 minutes
3. Resistance to enzymatic degradation

TABLE 8
Shape/cut scoring criteria

| Score | Shape | Cut |
|---|---|---|
| 0 | No observable shape retention; | Cut not possible. |
| | contents dissociate and | |
| | expand into the petri dish. | |
| | No self-assembly obvious. | |
| 1 | Minimal shape retention. | Able to cut, slightly more |
| | Loss of cylindrical shape. | viscous but cut sections |
| | Contents resemble | collapse; consistency resembles |
| | initial viscous slurry. | toothpaste. |
| 2 | Moderate shape retention. | Able to cut, slight deformation |
| | Cylindrical shape maintained | of cut slices with rounded |
| | with slight rounding of edges. | edges. |
| 3 | Significant shape retention. | Able to cut, complete retention |
| | Edges of the plug remain sharp | of shape in cut slices with well- |
| | and well defined. | defined edges |

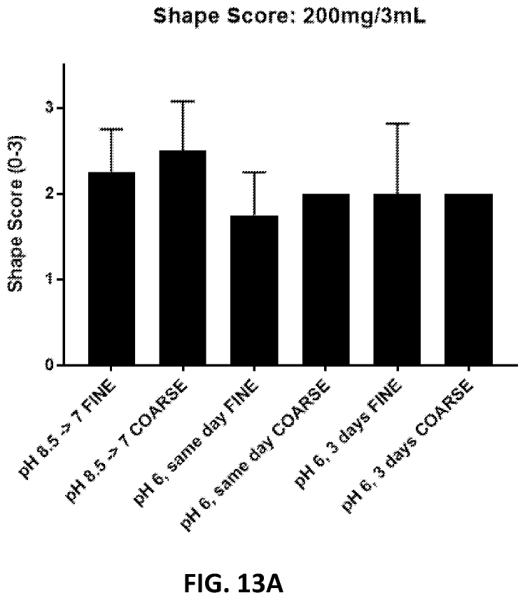

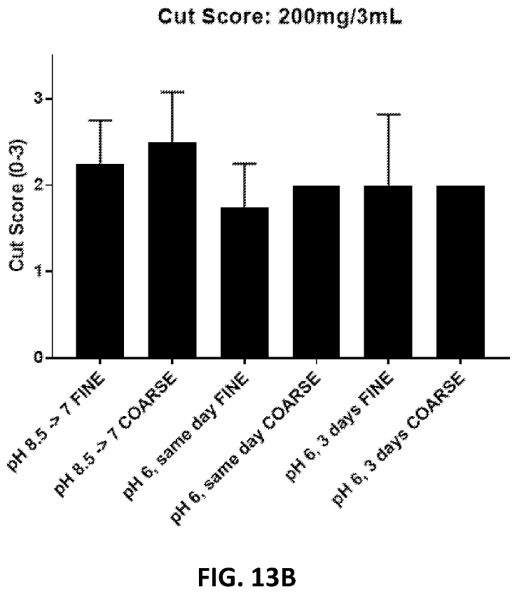

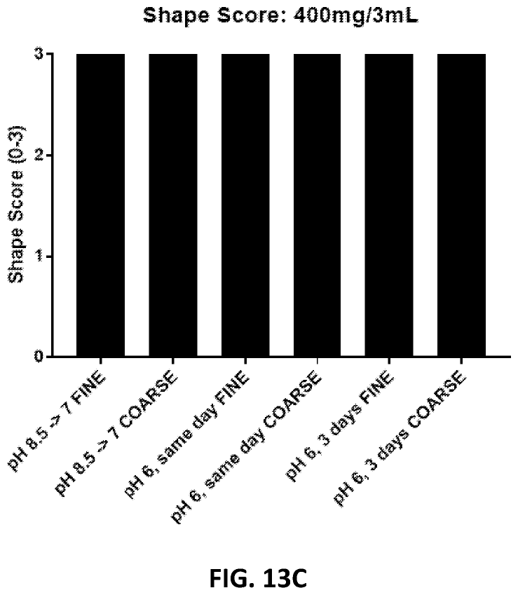

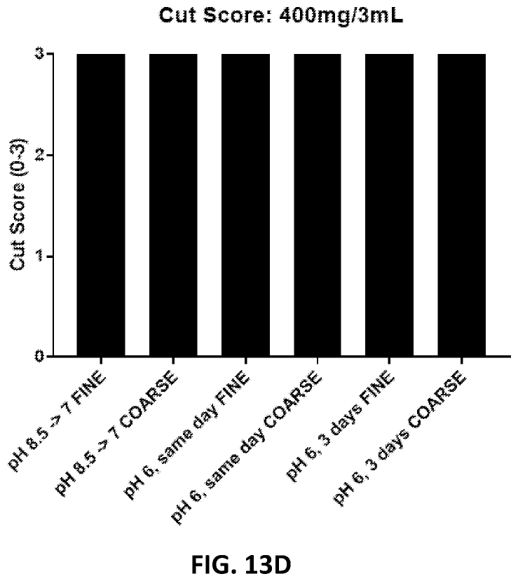

Semi-quantitative shape and cut scoring showed results like those found with the unsterilized powder in the studies described in Example 5. All groups at 400 mg/3 mL received the highest possible (3/3) shape scores (FIG. 13A) and cut scores (FIG. 13B), and had scores higher than those for corresponding samples prepared at 200 mg/3 mL, irrespective of neutralization method or particle size. For the 200 mg/3 mL samples, all groups had shape scores (FIG. 13C) and cut scores (FIG. 13D) of about 2. Thus, samples with a higher concentration of powder had improved shape retention.

### Compression Testing

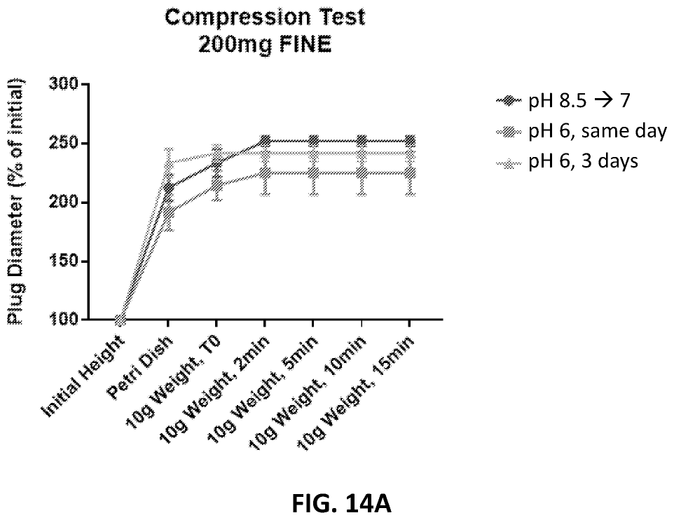

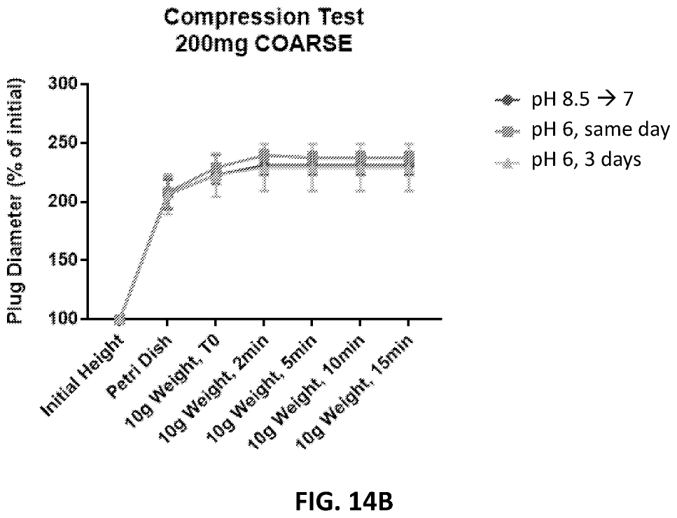

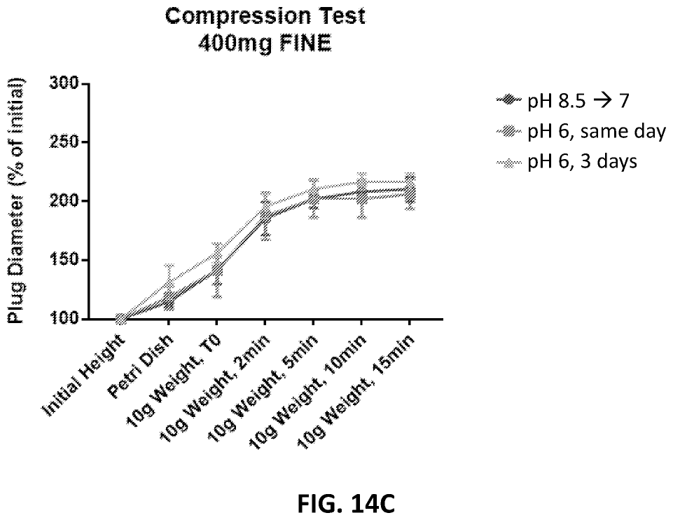

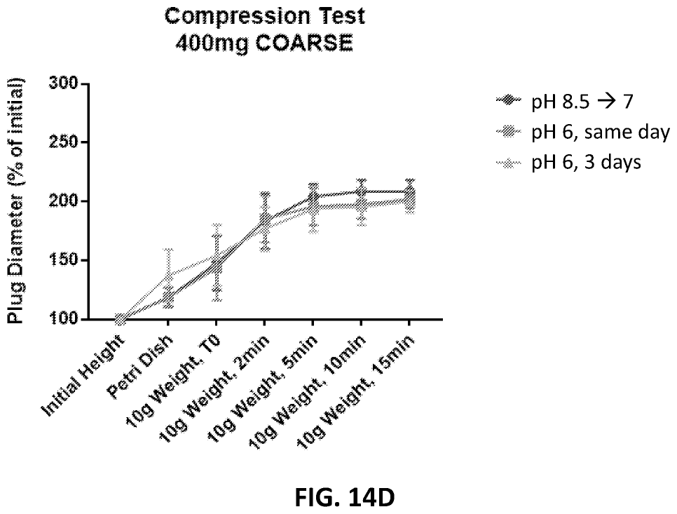

Samples at the higher powder concentration (400 mg/3 mL; FIGS. 14C and 14D) resisted compression better than samples at the lower concentration (200 mg/3 mL; FIGS. 14A and 14B) in all groups, with a noticeable lag in compression in the first two minutes under load. The maximum displacement for all groups was above 200%, compared to a maximum of 150% for the weakest non-sterilized group (200 mg/3 mL). Particle size and the pH adjustment method did not appear to significantly impact the ability of gels to resist compression.

### Resistance to Enzymatic Degradation

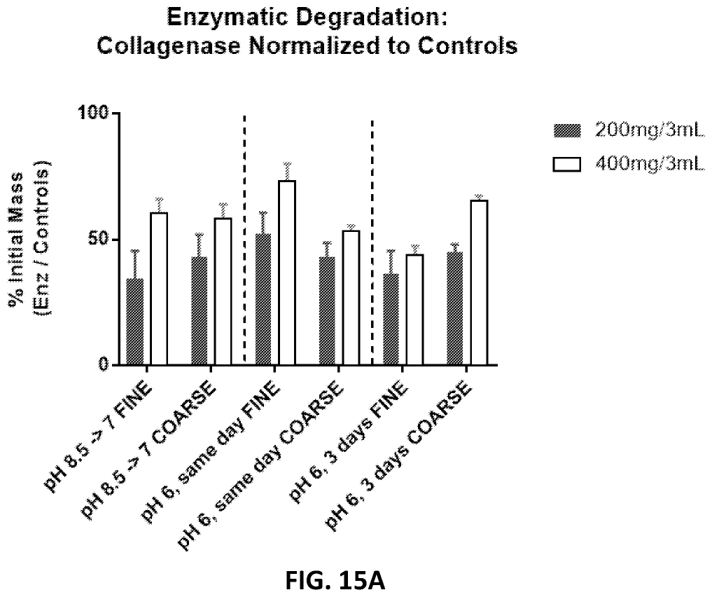

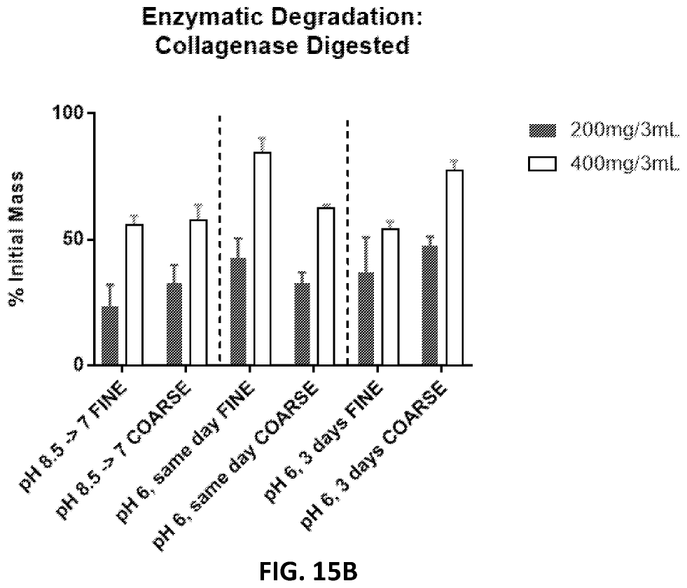

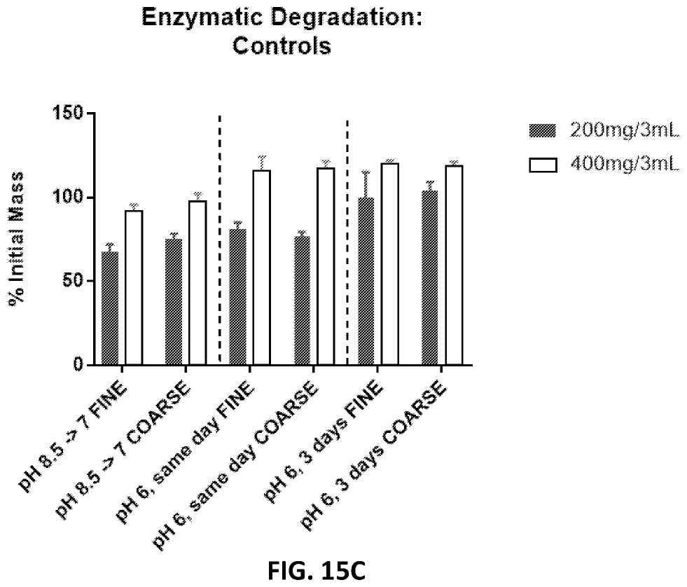

Sterilized samples containing the different powder concentrations were evaluated using the collagenase assay described above. The 400 mg/3 mL samples were more resistant to enzymatic degradation than the 200 mg/3 mL samples (FIGS. 15A-15C), irrespective of neutralization method and particle size. There did not appear to be a consistent trend between particle size and resistance to enzymatic degradation. However, in the pH 8.5→7 group, the coarse powder was more resistant to degradation, as was observed for the non-sterilized powder.

### Example 8—Mechanical Testing Using Instron

Additional studies were conducted to characterize the mechanical integrity of the gel more accurately when formed following hydration and homogenization with an iso-osmolar PBS solution using a vacuum mixing method. In particular, the mechanical integrity of the powder was studied by investigating three primary variables: (1) the powder particle size, (2) the pH adjustment/neutralization method following pepsin digestion, and (3) the ratio of powder to PBS (200 mg powder:3 mL PBS vs. 400 mg powder:3 mL PBS) during gel preparation. As described below, the results of these studies showed that the primary variable responsible for changes to the mechanical integrity of the gelled composite was the powder:PBS ratio. The results of the mechanical testing arm of the studies were crude, however, relying mostly on semi-quantitative shape/cut scoring based on qualitative observations, and compression testing that relied on the diameter increase of gel plugs of equal dimensions when subjected to loading under equal weights. By utilizing an INSTRON® ELECTROPULS™ E1000 (Instron; Norwood. MA) to measure the mechanical properties of the gel, more robust and accurate characterization can be achieved.

For these studies, the neutralization method and powder size were held constant (initial slurry pH of 2 was increased to pH 6.5 with the addition of a sodium hydroxide solution, and powder size was kept between 0.3 mm and 0.6 mm using a sieve), while the ratio of powder to PBS was 200 mg:3 mL or 400 mg:3 mL. PBS was used as an iso-osmolar replacement for whole blood in these tests, to isolate the mechanical contributions of gelation without the added contributions of clotting Target outcomes included elastic modulus, a stress relaxation curve, a dynamic compression curve, and Poisson's ratio (TABLE 9).

TABLE 9
Study design

GROUPS

| | 200 mg/ | 400 mg/ |
|---|---|---|
| TEST | 3 mL | 3 mL |
| Stiffness (Elastic Modulus) | N = 6 | N = 6 |
| Viscous Character (Stress Relaxation) | N = 6 | N = 6 |
| Fatigue (Dynamic Compression) | N = 6 | N = 6 |
| Deformation (Poisson's Ratio) | N = 6 | N = 6 |

Powder was manufactured as per PS-002 to PS-008 Revision B. Briefly, a mesodermal protein powder was made by decontaminating bovine elastic tissue with a solution containing hydrogen peroxide, decellularizing the tissue, and rinsing and lyophilizing the tissue. After lyophilization, the tissue was digested with pepsin in an acid solution, and the resulting slurry was neutralized, except that instead of neutralizing by pH adjustment to pH 8.5 and then back down to pH 7, the neutralizing method went from pH 2 to pH 6.5, to deactivate pepsin while avoiding premature pH-driven gelation. The powder was milled and sieved to create coarse particles with a controlled diameter between 0.3 mm and 0.6 mm. Loaded syringes were e-beam sterilized between 20-25 kGy.

Samples were prepared for each group by emptying the powder from terminally sterilized 500 mg syringes into a specimen container, to yield sufficient powder to fill four 5 mL syringes for each group (two syringes for the 200 mg/3 mL groups and four syringes for the 400 mg/3 mL groups). The 5 mL Luer lock syringes were filled with either 200 mg or 400 mg of powder, and the plunger was pulled to the 5 mL setting, making sure that no particles were caught between the plunger head and the syringe walls (as this could have interfered with pulling a vacuum during the mixing procedure). The plunger was held in place with a plastic cut-out. A 60 mL syringe was connected to the powder-filled syringe with a two-way valve. With the valve in the open position (parallel to the syringes), 60 mL of air was drawn into the 60 mL syringe, and the valve was twisted to the closed position (perpendicular to the syringes). This was repeated twice to create a vacuum within the 5 mL syringe. The 60 mL syringe was removed, leaving the valve in the closed position.

For PBS-powder mixing, a blunt 16-gauge needle was placed on the tip of an empty 5 mL syringe, and 3.3 mL of PBS was drawn up into the syringe (the extra 0.3 mL would be lost in the two-way valve). The syringe was turned upright, and air bubbles trapped at the plunger were loosened by tapping on the side of the syringe. All trapped air was ejected through the needle. Several drops of PBS were expelled into the open end of the two-way valve to remove air, and the syringe containing the powder was then connected to the valve. The valve was opened so that the PBS was drawn into the powder. The PBS syringe and the two-way valve were then removed, a syringe connector was connected to the powder+PBS filled syringe, and the empty PBS syringe was connected to the connector. The contents were mixed back and forth ten times, finally pushing all contents into the syringe initially containing the powder.

For gelation testing, syringes were placed into a water bath at 32° C. for two hours. After the two-hour incubation, one syringe at a time was removed from the water bath. An industrial razor blade was used to cut the tip off at the 0 mL mark, making sure to create a flat end of the slurry plug at the same time. One (1) mL of the gel was gently expelled onto a clear petri dish. An industrial razor blade was wetted in PBS and used to cut the plug. This was repeated to generate three cylindrical plugs from each syringe, and the plugs were oriented with their circular face parallel to the surface of the petri dish. Each plug was about 9 mm in length and 12 mm in diameter.

The plug to be mechanically tested was gently transferred to a small glass slide using forceps, and oriented with the circular face parallel to the surface of the slide. The initial height and diameter of the plug was measured and recorded. The glass slide was placed on the stand inside the INSTRON® and the machine door was secured. The load cell was balanced with the plug sitting uncompressed on the INSTRON® stand. The crosshead locks were securely tightened, and the strain gauge platform was at its lowest point. The actuator was lowered at full power until it lightly touched the plug. The strain gauge platform was raised and secured lightly against the strain gauge until the channel read approximately 0.1 mm. The strain gauge was balanced. The INSTRON® “Start” button was pushed to activate a program to compress the plug at a rate of 0.05 mm/s, until it reached a strain of 10%. The widest diameter of the compressed plug was measured and recorded when the test paused. The machine door was re-secured and the program was allowed to continue, performing a dynamic compression test (45 cycles at 1 Hz from 8-12% strain) and a stress relaxation test (constant 10% strain for 10 minutes). The data file was saved to an encrypted storage device for analysis.

The elastic modulus is a measure of stiffness, and was used to compare the resistance of gelled plugs to axial compression in the linear (elastic) region. Gel plugs were compressed at a uniform rate (0.05 mm/s) to a strain of 10% (roughly 0.8 mm). The modulus represented the ratio of stress (applied force/cross sectional area) divided by the strain (% length change compared to initial length).

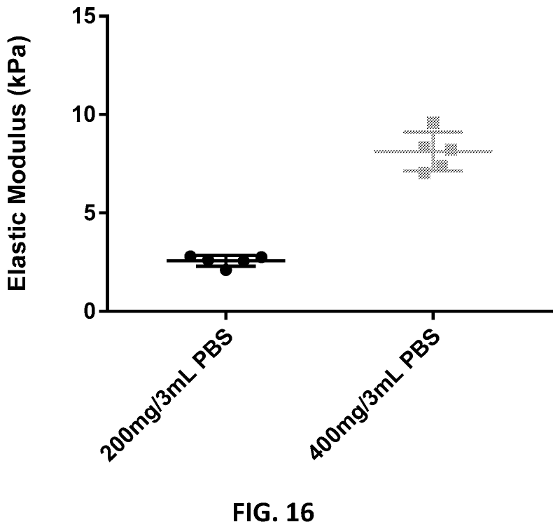

The mean elastic modulus of the 400 mg/3 mL gel was over three times greater than that of the 200 mg/3 mL gel (8.11±0.98 kPa vs. 2.57±0.27 kPa, mean±SD; TABLE 10 and FIG. 16). The elastic moduli of the 400 mg/3 mL gels were more variable than those of the 200 mg/3 mL gels, as indicated by a larger range and standard deviation around the mean (range: 2.54 vs. 0.68 standard deviation: 0.98 vs. 0.27). The difference in means was statistically significant, with a two tailed p value of <0.001 using an unpaired T-test with Welch's correction for unequal variance. Sample data distributions were assumed to be normal.

The dynamic compression test provided insight into the susceptibility of the hydrogels to fatigue. Under cyclic/oscillating loads, the internal structure of the hydrogels was progressively changed, caused by the formation and propagation of cracks in the polymer network, or by the loss and incomplete re-establishment of supporting non-covalent intermolecular bonding within the polymer/water network between each loading cycle. To monitor changes to the response to compression within hydrogels prepared at a concentration of 200 mg/3 mL PBS or 400 mg/3 mL PBS, each gel sample was subjected to 45 compression cycles at intervals of 1 Hz (1 load cycle per second) between strains of 8-12%. For each sample, an average of the maximum and minimum stress response readings across the first five load cycles were used as the “START” maximum and minimum stress response, while an average of the maximum and minimum stress readings across the last five load cycles (cycles 41-45) were used to represent the “END” range of stress responses. The difference in the START and END stress readings were calculated for each sample and used to represent the primary parameter of fatigue.

TABLE 10
Elastic modulus

| | 200 mg/3 mL PBS | 400 mg/3 mL PBS |
|---|---|---|
| Number of values | 5 | 5 |
| Units | kPa | kPa |
| Minimum | 2.11 | 7.04 |
| 25% Percentile | 2.34 | 7.21 |
| Median | 2.6 | 8.22 |
| 75% Percentile | 2.78 | 8.97 |
| Maximum | 2.79 | 9.58 |
| Range | 0.68 | 2.54 |
| Mean | 2.57 | 8.11 |
| Std. Deviation | 0.27 | 0.98 |
| Std. Error of Mean | 0.12 | 0.44 |
| Lower 95% CI | 2.23 | 6.89 |
| Upper 95% CI | 2.90 | 9.34 |

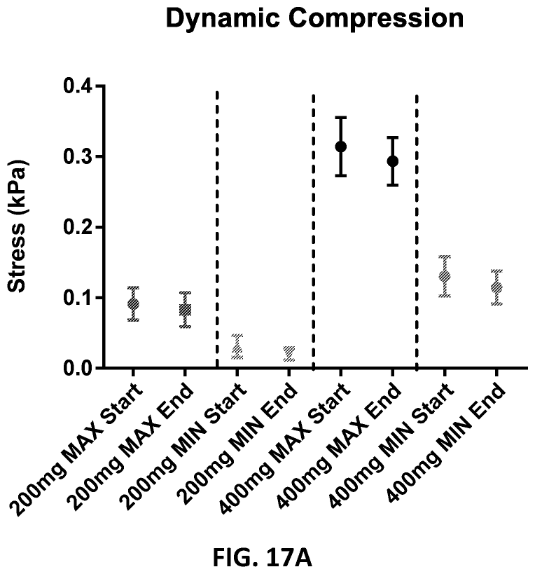

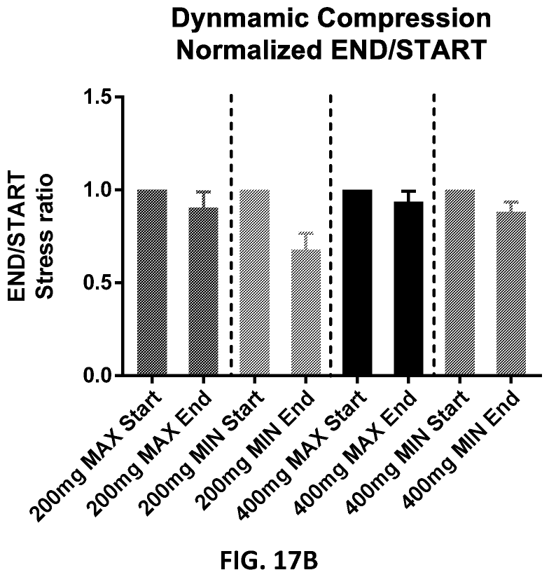

The stress response (resistance to compression) was higher in all 400 mg groups when compared to the corresponding 200 mg groups (FIG. 17A). The MAX Start and MAX End stress responses (stress response at 12% strain) were 3.4× and 3.5× greater in the 400 mg group vs. the 200 mg group, respectively, while the MIN Start and MIN End stress responses (8% strain) were 4.2× and 5.6× greater in the 400 mg group vs. the 200 mg group, respectively.

To compare differences in the START and END stress responses (fatigue) between corresponding 200 mg and 400 mg groups, the END stress responses were normalized to their respective START stress responses (e.g., the MAX End stress response average for the 200 mg group was normalized to the MAX Start stress response average for of the 200 mg group). The normalized values were then analyzed between 200 mg and 400 mg hydrogel groups (FIG. 17B). The decrease in normalized MAX stress response (stress response at 12% strain) was greater in the 200 mg powder/3 mL PBS hydrogel samples than the 400 mg powder/3 mL PBS samples, with a retention of 90.5% of the START stress vs. 93.6% respectively. Thus, the mechanical fatigue was greater in the 200 mg preparation. This difference was not statistically significant according to a one-way ANOVA with Tukey's correction for multiple comparisons (p-adjusted=0.896). A similar finding was observed in the fatigue at MIN stress responses (8% strain), with the 400 mg group retaining an average of 88.0% of its START stress response, while the 200 mg group only retained 67.6% of its START stress response. This difference was statistically significant (p-adjusted=0.002).

Stress relaxation is an import measure of mechanical character in viscoelastic materials such as hydrogels, where the elastic behavior of polymers is complemented by viscous flow within the hydrated network. When a constant external load is applied, a time dependent decrease in stress response is observed due to viscous flow within the gel. To monitor the stress relaxation in gels prepared with either 200 mg or 400 mg of powder, a 10% strain was applied and maintained, and the stress response was recorded across 10 minutes. The stress relaxation curves were modeled by a negative exponential function, with R² values ranging from 0.976 to 0.989:

Stress=a*e^{-bt}, where a is the initial stress at t=0 s and t is time in seconds.

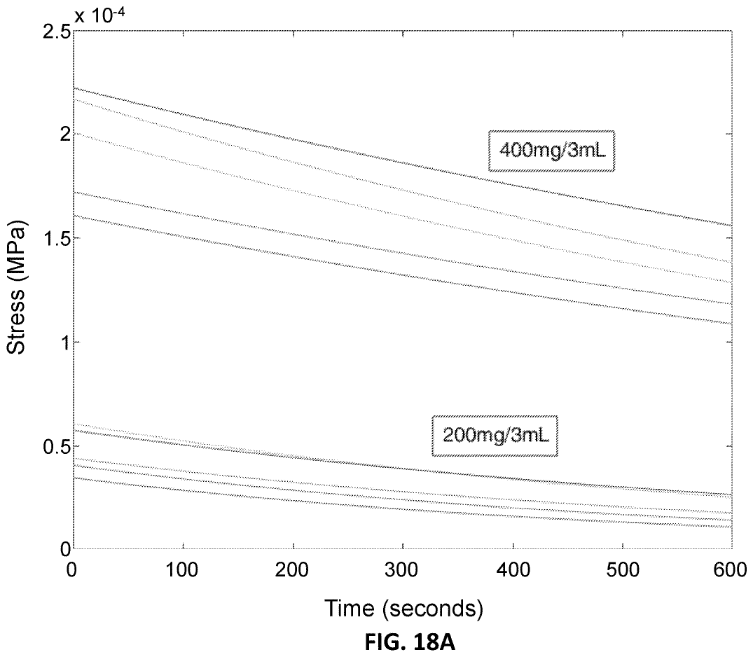

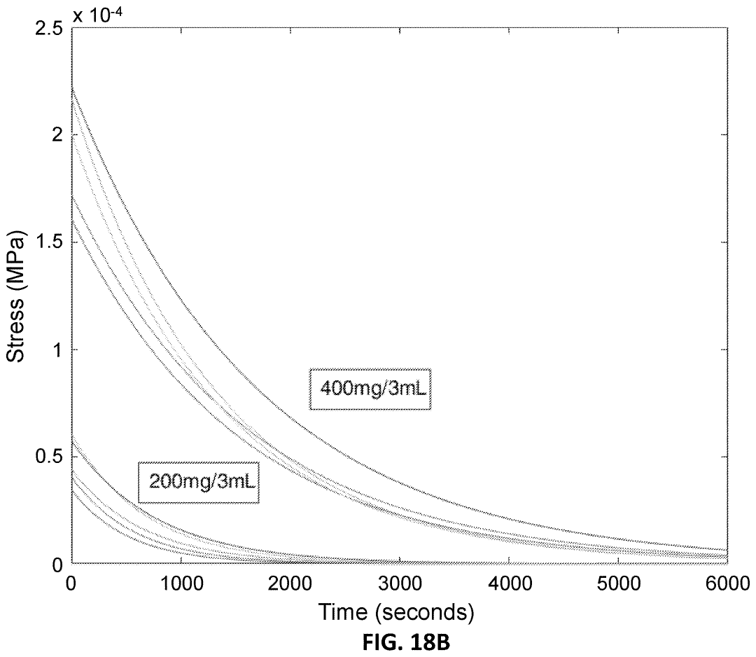

There was an observable difference in stress relaxation curves for hydrogels prepared with 200 mg of powder vs. those prepared with 400 mg of powder. FIG. 18A shows the stress response modeled through 10 minutes (the total time measured), and FIG. 18B extrapolates the predictive models through 100 minutes. The initial stress response was higher in the 400 mg hydrogels (0.19±0.03 kPa) than in the 200 mg hydrogels (0.05±0.01 kPa). At 10 minutes, the stress response in 400 mg hydrogels remained higher than the stress response in 200 mg hydrogels (0.13±0.02 kPa vs. 0.02 kPa±0.01 kPa respectively).

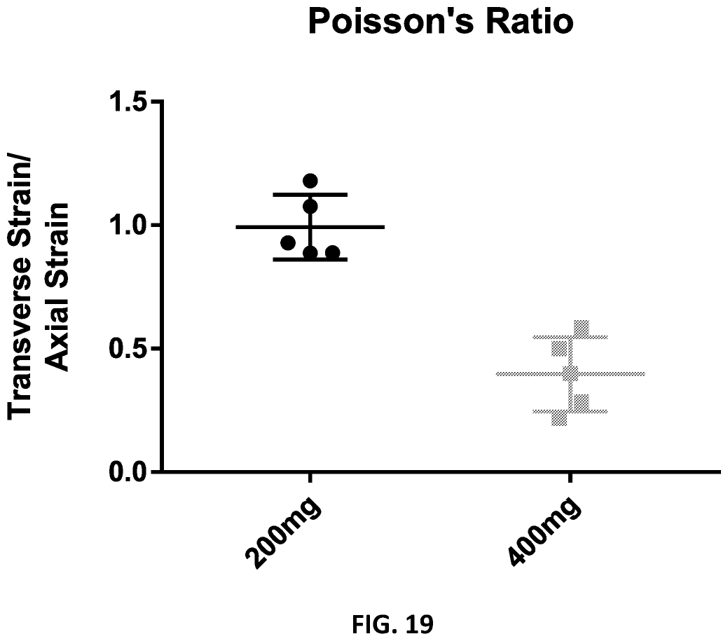

Poisson's ratio provides information about the deformation of materials under tensile or compressive loads. When the hydrogel plugs were subjected to compression in the axial direction, the plugs expanded in the transverse plane, perpendicular to the direction of loading. Poisson's ratio therefore was utilized as the ratio of transverse strain to axial strain; an axial strain of 10% was used in these studies. The deformation in the 200 mg hydrogels was significantly higher than in the 400 mg hydrogels, with average Poisson's ratios over twice as large (0.99±0.13 vs. 0.40±0.15 respectively; FIG. 19). This difference was statistically significant (p<0.001, unpaired t-test with Welch's correction for unequal variance).

Taken together, the above comprehensive mechanical testing demonstrated that hydrogel preparations containing 400 mg of powder mixed with 3 mL of PBS had significantly higher mechanical integrity than hydrogels prepared with 200 mg of powder per 3 mL of PBS. The 400 mg powder group was stiffer, with an elastic modulus over 3 times that of the 200 mg group. The 400 mg powder group also was more resistant to fatigue across 45 loading cycles, with stress responses over 2 times greater than those of the 200 mg group in the final stage of load cycling. Both groups had a viscous character, with stress relaxation curves sufficiently modeled by negative exponential functions, but the 400 mg group maintained greater stress readings across the measured 10 minute period, and was predicted to maintain a higher stress reading when functions were extrapolated to model up to 100 minutes. The 400 mg group also was more resistant to deformation under compressive loading, with a Poisson's ratio less than half that of the 200 mg group, indicating significantly less transverse strain when subjected to an axial strain of 10%.

### Example 9—Effect of Tissue Disinfection Agents on Characteristics of Mesodermal ECM-Derived Powder

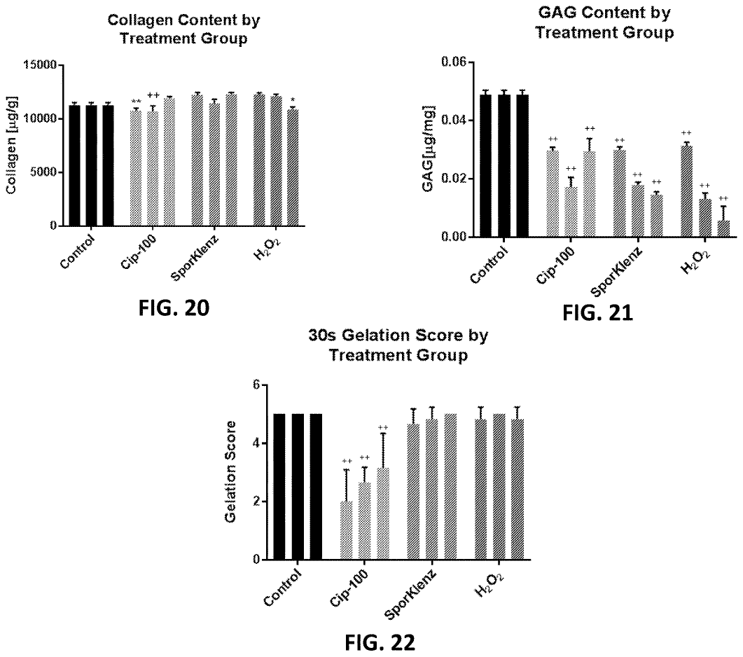

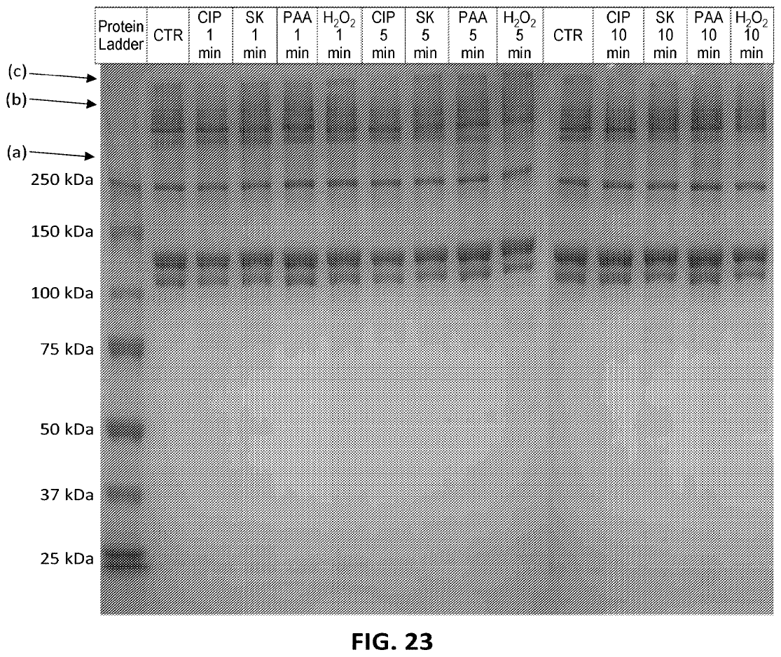

The effects of three different disinfection agents (CIP-100, SPOR-KLENZ®, and hydrogen peroxide) on ECM powder derived from mesodermal tissue were evaluated. SPOR-KLENZ® is a combination of 1% hydrogen peroxide, 0.08% peracetic acid, and less than 10% acetic acid. While the collagen content was unaffected by any of the disinfecting treatments (FIG. 20), each agent caused a significant loss of GAG in the product (FIG. 21). This loss of GAG did not appear to affect the gelation characteristics of the SPOR-KLENZ® or hydrogen peroxide agents (FIG. 22), or the protein content (FIG. 23).

### Example 10—Effect of Tissue Pretreatment Protocols on Reduction of Virus

For medical devices, a reduction in possible virus of 10⁶ (six logs) during manufacturing for the four classes of virus (enveloped DNA, enveloped RNA, non-enveloped DNA, and non-enveloped RNA viruses) is a feature desired by the FDA prior to product approval. This was successfully achieved for enveloped viruses (both DNA and RNA) with the combination of detergent and irradiation (see TABLE 11). For non-enveloped viruses such as REO3 (an RNA non-enveloped virus) and PPV (a DNA non-enveloped virus), the use of a detergent step in manufacturing was not helpful. In addition, while 15 kGy irradiation reduced REO3 by almost 10⁵ and an acid-pepsin step reduced REO3 by another 3 logs (thus a total of a six log reduction for the combined manufacturing process), reducing the viral load of PPV with pepsin/acid was ineffective. Thus, further studies were conducted to identify which of these processing steps would be useful for reducing PPV. These studies demonstrated that the use of hydrogen peroxide and PAA was effective in reducing PPV by about 4 logs, indicating that hydrogen peroxide and PPA in combination with terminal sterilization using e-beam (which reduced PPV by 2 logs at 15 kGy) would result in a six log reduction. The other steps were less effective. Thus, these studies showed that tissue pretreatment with a combination of hydrogen peroxide and PAA was effective against the parvovirus model, and did not appear to have a deleterious effect on the material.

TABLE 11
Virus removal/inactivation studies for specific manufacturing steps

| | Viral Clearance (log) | | | |
|---|---|---|---|---|
| Manufacturing Step | PRV* | PPV | BVDV | REO3 |
| Salt (x2) | | 0.29 (x2) | | 1.21 (x2) |
| Detergent | 5 | N/A | 2.8 | N/A |
| Acid/Pepsin | 5.2 | 0.6 | 4.5 | 3.56 |
| eBeam (15 kGy) | | 2.04 | | 4.82 |
| eBeam (20 kgy) | | | | |
| Tissue Pretreatment | | | | |
| 1% H₂O₂ 1 hour | | 1.22 | | |
| 5% Acetic acid 30 min | | 1.35 | | |
| 1% H₂O₂/ | | 3.93 | | |
| 0.05% PAA 10 min | | | | |
| 7.5% H₂O₂ 10 min | | 2.06 | | |
| Total Clearance | 10.2 | 3.22 + | 7.3 | 10.8 |
| | | 3.93 = 6+ | | |

*PRV, pseudorabies virus; PPV, porcine parvovirus; BVDV, bovine viral diarrhea virus; REO3, reovirus.

### Example 11—Effects of PAA Concentration on ECM-Derived Powder Characteristics

Since PAA had the greatest efficacy against PPV and was the only treatment that reduced PPV during manufacturing by six logs, studies were conducted to determine if varying the PAA concentration or time of exposure would result in changes in the physical characteristics of ECM-derived powder (including collagen concentration, GAG content and gelation). As indicated in TABLE 12, the following treatments were used: 0.10% PAA for 1 hour, 0.2% PAA for 1 hour, 0.1% PAA for 18 hours, 0.2% PAA for 18 hours, 0.2% PAA for 10 minutes, 7.5% hydrogen peroxide for 10 minutes, 1% CIP-100 for 10 minutes, and SPOR-KLENZ® (RTU) for 10 minutes.

TABLE 12
Study Design

| | # of | | |
|---|---|---|---|
| Condition | Samples | ID | Outcome Measure |
| 0.1% PAA for | 10 samples | 1-10 | 1. Collagen concentration. |
| 1 hour | | | 2. GAG concentration |
| | | | 3. Protein composition |
| | | | 4. Ability to gel |
| 0.2% PAA for | 10 samples | 11-20 | 1. Collagen concentration. |
| 1 hour | | | 2. GAG concentration |
| | | | 3. Protein composition |
| | | | 4. Ability to gel |
| 0.1% PAA for | 10 samples | 21-30 | 1. Collagen concentration. |
| 18 hours | | | 2. GAG concentration |
| | | | 3. Protein composition |
| | | | 4. Ability to gel |
| 0.1% PAA for | 10 samples | 31-40 | 1. Collagen concentration. |
| 18 hours | | | 2. GAG concentration |
| | | | 3. Protein composition |
| | | | 4. Ability to gel |
| Control | 10 samples | 41-50 | 1. Collagen concentration. |
| | | | 2. GAG concentration |
| | | | 3. Protein composition |
| | | | 4. Ability to gel |
| 7.5% hydrogen | 10 samples | 51-60 | 1. Collagen concentration. |
| peroxide | | | 2. GAG concentration |
| 10 min | | | 3. Protein composition |
| | | | 4. Ability to gel |
| 1% CIP-100 | 10 samples | 61-70 | 1. Collagen concentration. |
| 10 min | | | 2. GAG concentration |
| | | | 3. Protein composition |
| | | | 4. Ability to gel |
| SPOR-KLENZ ® | 10 samples | 71-80 | 1. Collagen concentration. |
| (RTU) | | | 2. GAG concentration |
| 10 min | | | 3. Protein composition |
| | | | 4. Ability to gel |
| 0.2% PAA | 10 samples | 81-90 | 1. Collagen concentration. |
| 10 min | | | 2. GAG concentration |
| | | | 3. Protein composition |
| | | | 4. Ability to gel |

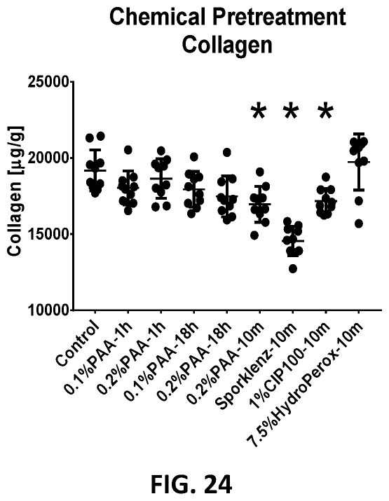

Collagen concentration: The average collagen concentration and standard deviation for each treatment are provided in TABLE 12 and FIG. 24. Chemical pre-treatment with 0.2% PAA, SPOR-KLENZ®, or CIP-100 for 10 minutes resulted in a significantly lower collagen concentration when compared to the control group (p-adj=0.0314).

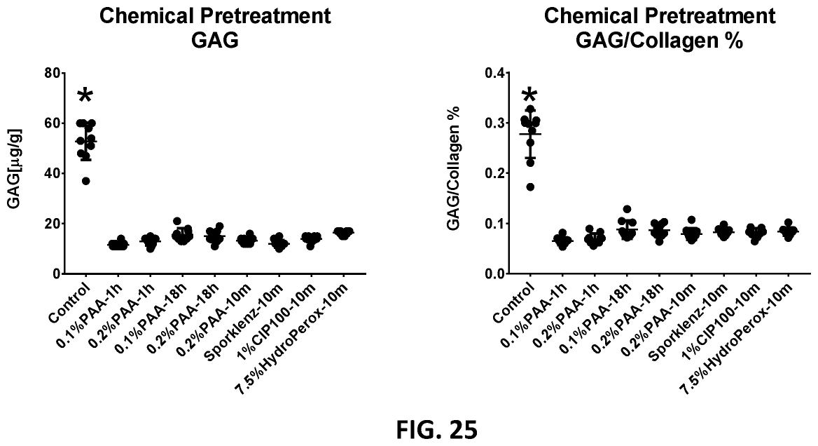

GAG Content: Average GAG concentration and standard deviation, as well as GAG content per collagen content in percent, are reported in TABLE 14 and FIG. 25. All chemical pre-treatment groups showed a significant reduction in absolute GAG content and GAG/collagen percentage when compared to the control group (p-adj.<0.0001 for all comparisons).

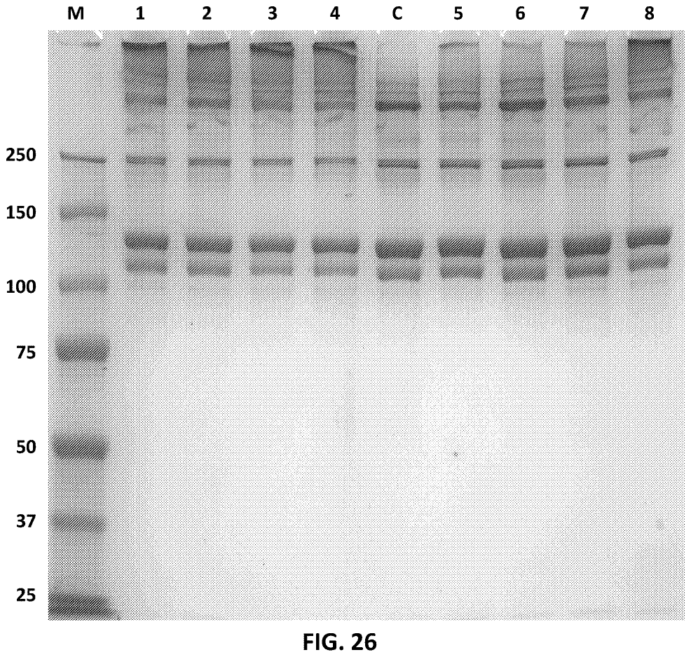

Protein Composition: Visual comparison of the location of the protein bands suggested that there were no significant changes in the molecular weight of the individual proteins (FIG. 26). However, the PAA chemical treatment groups appeared to show stronger staining in the upper section of the gel, which would be consistent with less complete pepsin digestion of the source tissue.

TABLE 13
Collagen concentration in ECM-
derived powder for PAA treatments

| | Avg. Collagen | Standard |
|---|---|---|
| | Conc. | Deviation |
| Group | (μg/g) | (μg/g) |
| 0.1% PAA 1 hour | 18039 | 1112 |
| 0.2% PAA 1 hour | 18645 | 1289 |
| 0.1% PAA 18 hours | 17943 | 1166 |
| 0.2% PAA 18 hours | 17478 | 1340 |
| Control | 19176 | 1344 |
| 7.5% hydrogen peroxide 10 min | 19734 | 1835 |
| 1% CIP-100 10 min | 17169 | 817 |
| SPOR-KLENZ ® (RTU) 10 min | 14550 | 966 |
| 0.2% PAA 10 min | 16959 | 1179 |

TABLE 14
GAG concentrations with various PAA treatments

| | Avg. GAG | Standard | GAG/ |
|---|---|---|---|
| | Conc. | Deviation | Collagen |
| Group | (μg/g) | (μg/g) | % |
| 0.1% PAA 1 hour | 11.6 | 0.9661 | 0.06461 |
| 0.2% PAA 1 hour | 12.9 | 1.595 | 0.06951 |
| 0.1% PAA 18 hours | 15.6 | 2.503 | 0.08767 |
| 0.2% PAA 18 hours | 15.1 | 2.331 | 0.08655 |
| Control | 52.8 | 7.406 | 0.2775 |
| 7.5% Hydrogen Peroxide 10 | 16.4 | 0.8433 | 0.0837 |
| min | | | |
| 1% CIP-100 10 min | 14 | 1.247 | 0.08172 |
| SPOR-KLENZ ® (RTU) 10 | 12 | 1.491 | 0.0824 |
| min | | | |
| 0.2% PAA 10 min | 13.3 | 1.337 | 0.07895 |

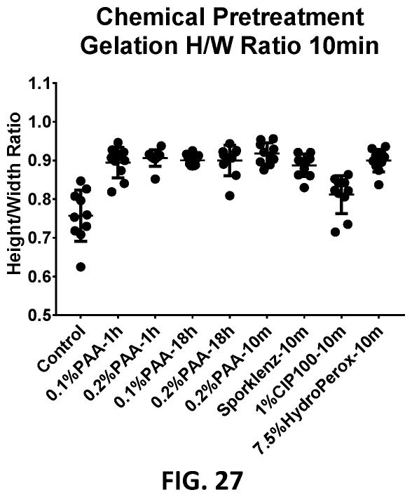

Gelation ability: There was a significant improvement in the gelation width/height ratio, the cut resistance at 10 minutes and the shape stability 5 minutes after cutting in the chemically pre-treated samples when compared to the control group (TABLE 15 and FIG. 27: p-adj<0.0001 for all comparisons).

TABLE 15
Gelation characteristics with the various PAA protocols.

| | Gelation | | Shape |
|---|---|---|---|
| | Height/Width | Cut | Stability |
| | Ratio | Resistance | 5 min |
| | after | at 10 min | after |
| Group | 10 min | (0-5) | cut |
| 0.1% PAA 1 hour | 0.8942 | 5 | 5 |
| 0.2% PAA 1 hour | 0.9062 | 5 | 5 |
| 0.1% PAA 18 hours | 0.9003 | 5 | 5 |
| 0.2% PAA 18 hours | 0.8997 | 5 | 5 |
| Control | 0.757 | 3.7 | 3.3 |
| 7.5% Hydrogen | 0.8995 | 4.6 | 4.7 |
| Peroxide 10 min | | | |
| 1% CIP-100 10 min | 0.8115 | 4 | 3.7 |
| SPOR-KLENZ ® (RTU) | 0.8873 | 4.7 | 4.7 |
| 10 min | | | |
| 0.2% PAA 10 min | 0.9176 | 5 | 5 |

These studies demonstrated that chemical pre-treatment of the source tissue with all PAA protocols led to a significant decrease in GAG content as compared to the control samples. This was not different between groups, suggesting that the GAG loss likely occurred within first ten minutes of exposure.

The loss of GAG did not affect the ability of the ECM-derived powder to gel, which is the key function of the material. In fact, the samples treated with PAA had better maintenance of the gel shape over time. This unexpected result was apparent from the reduced amount of height loss of the gel cylinder over 10 minutes, the increased resistance during cutting of the gel, and the subsequent shape stability of the cut gel. Moreover, this was true for exposures to PAA ranging from 10 minutes at low concentration (0.08% PAA with 1% hydrogen peroxide and <10% acetic acid—the SPOR-KLENZ® group) up to 18 hours at 0.2% PAA.

However, the increasing time of exposure of the tissue to PAA and use of a higher concentration of PAA led to a change in the ability of the pepsin to solubilize the collagen, as noted in gel electrophoresis studies in which the PAA-only groups had more staining visible in the upper portions of the gel bands where proteins with a higher molecular weight are located. While the protein band patterns overall appeared unchanged, the higher intensity staining for large molecules and lower intensity staining for small molecules may indicate a less complete breakdown of the collagen proteins during pepsin digestion, which would be consistent with the finding that the PAA-treated tissues took longer to digest than control samples.

Taken together, these studies demonstrated that the use of 0.1% PAA for 10 minutes, combined with 1% hydrogen peroxide (as seen in the SPOR-KLENZ® group) led to improved gelation and did not interfere with pepsin solubilization, and also led to an almost 4 log reduction in parvovirus.

### Example 12—Effects of Anticoagulant Use In Vitro

An in vitro study of the reactivation of coagulation for anticoagulated blood demonstrated that the addition of calcium to the anticoagulated blood reestablished the ability of the blood to coagulate. However, blood anticoagulated using sodium citrate (NaC) clotted more quickly than blood anticoagulated with acid citrate dextrose (ACD). In addition, lower concentrations of calcium chloride appeared to recover coagulation of the anticoagulated blood more quickly. All CaCl₂) amounts tested (8 mg, 10 mg, and 12 mg, equivalent to 1 mL of 72 mM, 90 mM, and 108 mM CaCl₂) solution) were able to rescue clotting of 4 mL of anticoagulated blood in all groups (TABLE 16). In Group 1, the various concentrations of CaCl₂) solutions were used to rehydrate the mesodermal protein powder directly prior to mixing with the anticoagulated blood. In Group 2, the equivalent dry weights of CaCl₂) were mixed with the mesodermal protein powder prior to mixing the powder with blood. In Group 3, the various CaCl₂) solutions were dried in the syringes prior to addition of the mesodermal protein powder. Coagulation was initiated more quickly in the NaC anticoagulated blood than in ACD anticoagulated blood in all three groups. Clotting times in the NaC groups were faster in Group 1 when rehydrated with the lower and middle concentrations of CaCl₂), suggesting that an optimal concentration may exist, although no difference in time to clot was observed in Groups 2 and 3 for NaC anticoagulated blood. The highest CaCl₂) concentration resulted in the fasted time to clot in the ACD anticoagulated blood, consistent with the fact that the theoretical mass of CaCl₂) needed to saturate the free citrate was closest to the highest concentration tested (12 mg). It is to be noted that the ACD and NaC anticoagulated blood used in these studies came from different donors, so natural variability in coagulation kinetics between individuals was likely to affect the results.

TABLE 16A
Coagulation results

| | | Time | | Time |
|---|---|---|---|---|
| | Sample | to clot | Sample | to clot |
| | (NaC) | (min) | (ACD) | (min) |
| Group 1 | S1-L | 6 | A1-L | 14 |
| | S1-M | 6 | A1-M | 10 |
| | S1-H | 8 | A1-H | 10 |
| Group 2 | S2-L | 6 | A2-L | 32 |
| | S2-M | 6 | A2-M | 12 |
| | S2-H | 6 | A2-H | 10 |
| Group 3 | S3-L | 8 | A3-L | 14 |
| | S3-M | 8 | A3-M | 12 |
| | S3-H | 8 | A3-H | 10 |

Further studies were conducted to determine the optimal concentration of calcium chloride to use as a rehydration solution with ACD anticoagulated blood to recover the ability of blood to clot prior to injection. The ratio of rehydration solution to blood was set to mimic that to be used in the preparation of the final ECM-derived powder composite (1 part rehydrated powder to 2 parts blood by volume). The solutions were prepared as indicated in TABLE 16B.

Human whole blood was drawn and mixed with 10% ACD or 15% ACD. 2 mL of each anticoagulated blood sample was aliquoted into two serum collection tubes coated with a clotting activator. 1 mL of a dilute calcium chloride solution (either 41 mM or 59 mM) was added to each ACD-blood group to create the matrix in TABLE 16C. The time from mixture to clotting was observed and recorded in TABLE 16C.

The coagulation time of blood not anticoagulated with ACD was 6 minutes. As shown in TABLE 16C, for both ACD preparations, the lower CaCl₂) concentration surprisingly resulted in faster clotting, and the fastest time to clot was observed in the 10% ACD mixed with 41 mM CaCl₂). Thus, the 41 mM solution of CaCl₂), mixed in a 1:9 ratio with anticoagulated blood, successfully recovered coagulation ability. This calcium chloride solution and mix ratio was also used in the guinea pig study that used anticoagulated blood for ECM-derived powder rehydration and intra-articular injection (Example 13).

TABLE 16B

| | | Prep for 15% ACD |
|---|---|---|
| | Prep for 10% ACD | (1.5:8.5 ACD to blood |
| | (1:9 ACD to blood | ratio) |
| Dilute CaCl₂ Rehydration Solution | ratio) | Ratio used in BD |
| Preparation | Used in PRP prep | vacutainers |
| Prepare 100 mM stock CaCl₂ solution | Dissolve 5.55 g CaCl₂ in 500 mL sterile | |
| | water | |
| | Sterile filter | |
| Moles of free citrate provided by each | 47 mmol | 70 mmol |
| ACD preparation | | |
| *HUMAN DOSE* | | |
| Moles of CaCl₂ needed to provide | 70 mmol | 105 mmol |
| enough free Ca²⁺ to saturate the free | | |
| citrate ions from the ACD (free citrate x | | |
| 1.5) *HUMAN DOSE* | | |
| Mass of CaCl₂ corresponding to the | 9 mg | 13 mg |
| moles needed (rounded up to nearest | | |
| mg) *HUMAN DOSE* | | |
| Concentration of dilute CaCl₂ to use as | 41 mM | 59 mM |
| rehydration solution to provide mass of | | |
| CaCl₂ needed (rounded up to nearest | | |
| mM) | | |
| Prepare dilute CaCl₂ for rehydration of | Dilute 16.4 mL of 100 | Dilute 23.6 mL of 100 |
| powder given the concentration needed | mM CaCl₂ Stock | mM CaCl₂ stock |
| | solution in 23.6 mL of | solution in 16.4 mL of |
| | sterile water to create | sterile water to create |
| | 41 mM CaCl₂ solution | 59 mM CaCl₂ solution |

TABLE 16C
Time to Clot

| | 10% ACD | 15% ACD |
|---|---|---|
| | in blood | in blood |
| 41 mM CaCl₂ (Osmolarity: 107 mOsm) | 6 min | 9 min |
| 59 mM CaCl₂ (Osmolarity: 152 mOsm) | 9 min | 10 min |

### Example 13—Effects of Anticoagulant Use In Vivo

A live animal, in vivo study of the product that can accommodate for use of anticoagulated blood in the Guinea pig model was conducted. Gait analysis and micro-CT analysis of the subchondral bone were evaluated, and no detrimental effects related to the use of anticoagulated blood as an additive to the ECM-derived powder were observed. In brief, no statistically significant differences were observed in the hind limb loading side-to-side difference between the animal groups treated with injections of mesodermal protein powder mixed with either anticoagulated or fresh blood. Similarly, the subchondral bone density as an indicator for osteoarthritic changes did not show any significant differences between the groups.

### Example 14—Effect of Moisture-Proof Packaging

A foil/film construct was determined to be a suitable moisture-proof packaging option. In accordance with industry standards, a burst and bubble test, as well as peel testing of the seal were performed. The peel force results indicated that the seals in three consecutive sealing runs were consistent (TABLE 17), indicating acceptable operating and performance quality.

TABLE 17
Peel force results

| | Run 1 (optimal - | Run 2 (optimal - | Run 3 (optimal - |
|---|---|---|---|
| | setting 7) | setting 7) | setting 7) |
| Minimum | 1.45 | 1.46 | 1.61 |
| Maximum | 3.29 | 3.44 | 3.69 |
| Mean | 2.81 | 2.88 | 2.82 |
| Std. Dev. | 0.5 | 0.44 | 0.52 |
| Mean + 3 SD | 4.31 | 4.20 | 4.38 |
| Mean − 3 SD | 1.31 | 1.56 | 1.26 |

### Example 15—Product Characterization (Quantitative MS/ELISA/SDS-PAGE)

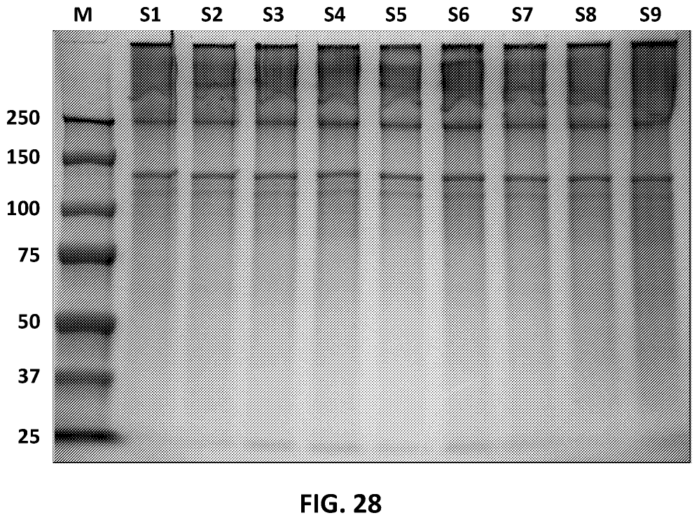

To further characterize the ECM-derived powder protein composition, an SDS-PAGE analysis was performed (FIG. 28). Strong bands were distinguishable where collagen type 1 dimer (270 kDa) and collagen type 1 monomers (130 kDa) were expected, highlighting that the main protein of the powder was collagen. Mild smearing was visible around and below the bands, indicative of protein breakdown due to the electron beam irradiation.

A more detailed protein analysis of the powder was done using mass spectroscopy. A total of 1072 peptides were identified. Using 032201 aseptic powder as the control (since it was derived from historically used tissue), the output list was sorted from highest to lowest relative peak intensity for this group. The top 20 peptides (mean, SD) are listed in TABLE 18. For this analysis, individual peptides of fibrillar proteins and subunits for those with higher quaternary structures were considered individually. Type I collagen was the most abundant peptide identified in each group, followed by Type III collagen and then small amounts of type II collagen, fibrillins 1 and 2, and keratins.

TABLE 18
Top 20 most abundant peptides

| *n = 6 all groups | | A 112101 e-beam | |
|---|---|---|---|
| Identified proteins (1072) | Alternate ID | AVG | STD DEV |
| Collagen alpha-1(I) chain | COL1A1 | 39.69% | 1.97% |
| Collagen alpha-2 (I) chain | COL1A2 | 33.61% | 2.50% |
| Collagen alpha-1(III) chain | COL3A1 | 8.20% | 1.19% |
| Collagen alpha-1(III) chain | COL3A1 | 6.52% | 3.29% |
| Collagen alpha-1(II) chain | COL2A1 | 3.95% | 0.26% |
| Cytokeratin-1 | KRT1 | 0.84% | 0.23% |
| Fibrillin-1 | FNB1 | 0.74% | 0.16% |
| Collagen type VI alpha 3 chain | COL6A3 | 0.64% | 0.19% |
| Keratin 10 | KRT10 | 0.47% | 0.10% |
| Keratin 2 | KRT2 | 0.56% | 0.31% |
| Collagen type V alpha 2 chain | COL5A2 | 0.22% | 0.04% |
| Collagen type V alpha 1 chain | COL5A1 | 0.26% | 0.02% |
| Keratin 3 | KRT3 | 0.22% | 0.12% |
| KRT5 protein | KRT5 | 0.26% | 0.12% |
| Keratin, type II cytoskeletal 75 | KRT75 | 0.20% | 0.12% |
| Fibrillin 2 | FBN2 | 0.33% | 0.15% |
| Keratin 6A | KRT6A | 0.19% | 0.08% |
| Pancreatic trypsin inhibitor | BPTI | 0.10% | 0.03% |

### Example 16—Identification and Validation of Sterilization Method (Supercritical CO₂)

No improvement in histological cartilage breakdown or gait was observed in a single and multi-dose injection study in the guinea pig spontaneous osteoarthritis model when using electron beam sterilized ECM-derived powder. Thus, supercritical CO₂ was evaluated as an alternative sterilization method. A comparison of electron beam sterilized and scCO₂ sterilized ECM-derived powder also was conducted, including evaluation of collagen content, GAG content, DNA content, phospholipid content, pepsin activity, protein content, resistance to enzymatic degradation, and physical characteristics.

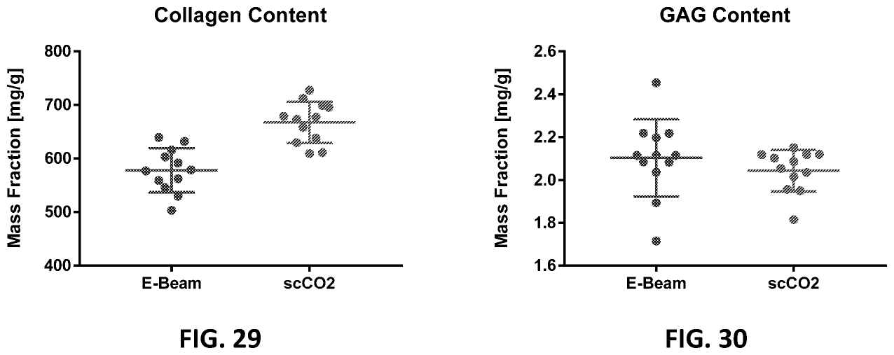

Collagen content: A Sircol collagen assay was conducted using two samples from six e-beam sterilized syringes (total n=12, 20-25 kGy) and twelve samples of powder sterilized using scCO₂ to compare the dry mass fraction of collagen in powder manufactured from the same lot following each form of terminal sterilization. Each sample was prepared by dissolving 30±3 mg of powder in 50 mL of dilute aqueous hydrochloric acid (HCl) at pH 2 (n=12 for each group). The mean mass fractions of collagen in dry ECM-derived powder sterilized with either E-Beam or scCO₂ were 578 mg/g and 668 mg/g, respectively, corresponding to dry weight (w/w) percentages of 58% and 67% (FIG. 29). The 9% difference in total dry weight percentage was statistically significant according to an unpaired, two-tailed t-test with Welch's correction with a p value of <0.001. The variability of each group was similar, with standard deviations in the E-Beam and scCO₂ groups of 41 and 39 mg/g, respectively.

GAG content: A Blyscan GAG assay was conducted using two samples from six E-Beam sterilized syringes (total n=12, 20-25kGy) and twelve samples of powder sterilized using ScCO₂ to compare the dry mass fraction of glycosaminoglycan in powder manufactured from the same lot following each form of terminal sterilization. Each sample was prepared by dissolving 30±3 mg of powder in 1 mL of a papain buffer solution (n=12 for each group). The mean mass fractions of GAG in dry ECM-derived powder sterilized with either E-Beam or scCO₂ were 2.1 mg/g and 2.0 mg/g, respectively, corresponding to dry weight (w/w) percentages of 2.1% and 2.0% (FIG. 30). The 0.1% difference in total dry weight percentage was not statistically significant according to an unpaired, two-tailed t-test with Welch's correction with a p value of 0.323. The variability was larger in the E-Beam group with standard deviations in the E-Beam and scCO₂ groups of 0.18 and 0.10 mg/g, respectively.

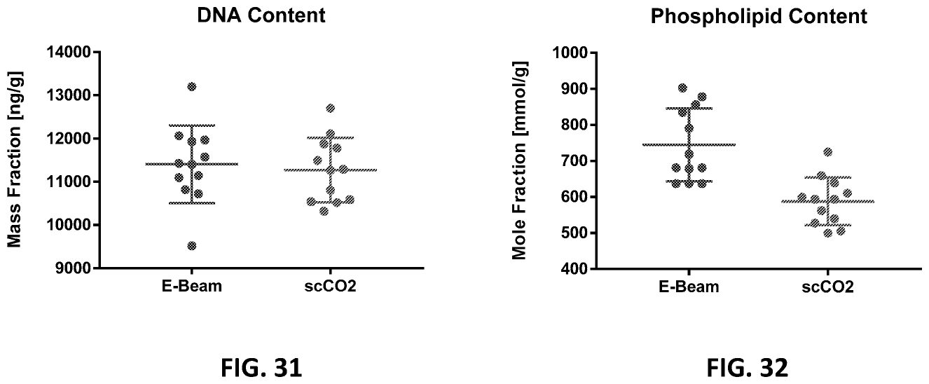

DNA content: A Picogreen dsDNA assay was carried out using two samples from six E-Beam sterilized syringes (total n=12, 20-25kGy) and twelve samples of powder sterilized using scCO₂ to compare the dry mass fraction of DNA in powder manufactured from the same lot following each form of terminal sterilization. Each sample was prepared by dissolving 30±3 mg of powder in 1 mL of a papain buffer solution (n=12 for each group). The DNA content was determined as a measure of effective decellularization and subsequent removal of residual cellular material from the extracellular matrix (ECM), and 50,000 ng/g was considered to be the acceptable limit. The mean mass fractions of DNA in dry ECM-derived powder sterilized with either E-Beam or scCO₂ were 11,406 ng/g and 11,275 ng/g, respectively, corresponding to dry weight (w/w) percentages of 0.00114% and 0.00113% (FIG. 31). The difference in total dry weight percentage was not statistically significant according to an unpaired, two-tailed t-test with Welch's correction with a p value of 0.700. The variability of each group was similar but slightly larger in the E-Beam group, with standard deviations in the E-Beam and scCO₂ groups of 896 and 746 ng/g, respectively.

Phospholipid Content: An ENZYCHROM™ phospholipid assay was carried out using two samples from six E-Beam sterilized syringes (total n=12, 20-25kGy) and twelve samples of powder sterilized using scCO₂ to compare the dry mole fraction of phospholipid in powder manufactured from the same lot following each form of terminal sterilization. Each sample was prepared by dissolving 10±1 mg of powder in 1 mL of a tris-buffered Triton-X 102 solution (n=12 for each group). Phospholipid content is a secondary measure of effective decellularization and subsequent removal of residual cellular material from the extracellular matrix. The mean mole fractions of phospholipid in dry ECM-derived powder sterilized with either E-Beam or scCO₂ were 745 mmol/g and 588 mmol/g, respectively (FIG. 32). The 157 mmol/g difference in mole fractions was statistically significant according to an unpaired, two-tailed t-test with Welch's correction with a p value of <0.001. The variability was slightly larger in the E-Beam group with standard deviations in the E-Beam and scCO₂ groups of 101 and 66 mmol/g, respectively.

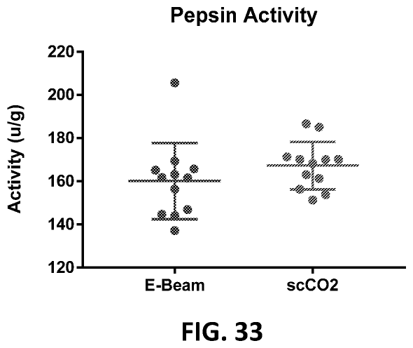

Pepsin activity: Two samples from six E-Beam sterilized syringes (total n=12, 20-25kGy) and twelve samples of powder sterilized using scCO₂ were used to compare the residual activity of pepsin in powder manufactured from the same lot following each form of terminal sterilization. Each sample was prepared by dissolving 20±2 mg of powder in 1 mL of dilute pH 2 HCl (n=12 for each group). The mean residual activities of pepsin in dry ECM-derived powder sterilized with either E-Beam or scCO₂ were 160 u/g and 167 u/g, respectively (FIG. 33). The 7 u/g difference in mole fractions was not statistically significant according to an unpaired, two-tailed t-test with Welch's correction with a p value of 0.250. The variability was slightly larger in the E-Beam group with standard deviations in the E-Beam and scCO₂ groups of 18 and 11, u/g respectively.

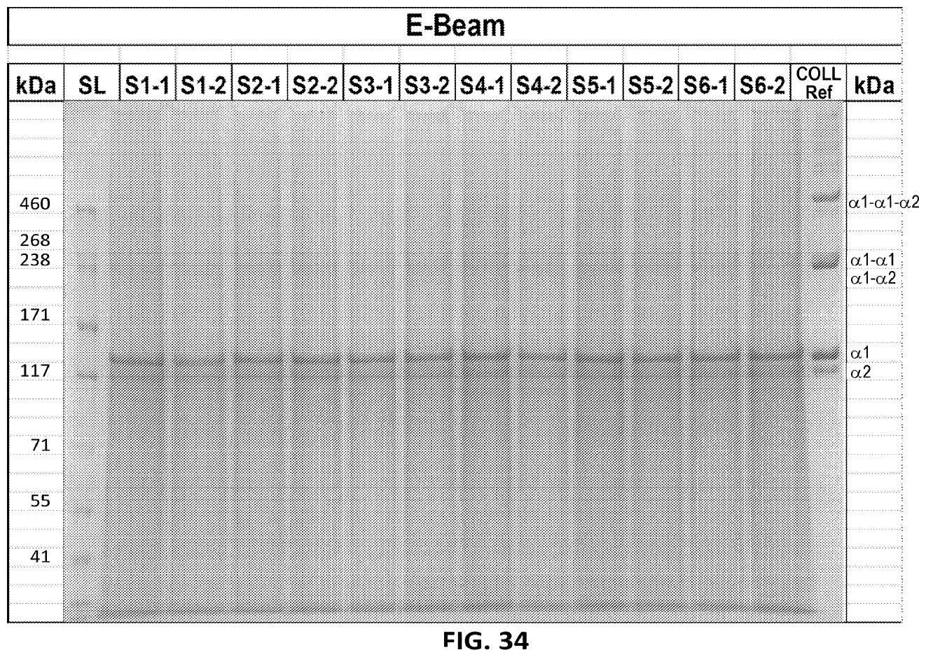

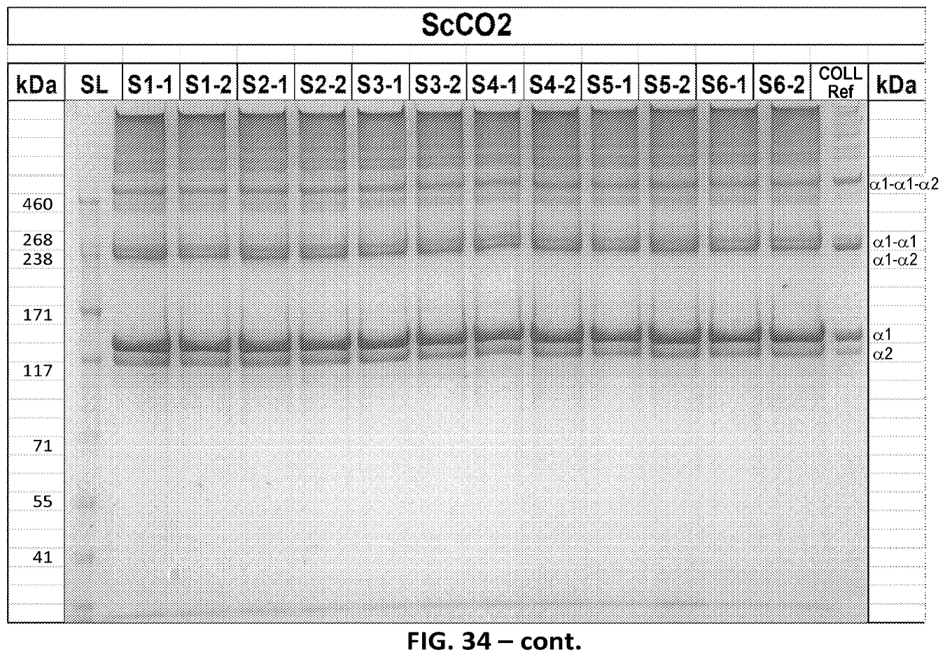

SDS-PAGE: Two samples from six E-Beam sterilized syringes (total n=12, 20-25kGy) and twelve samples of powder sterilized using scCO₂ were used to compare the protein banding patterns in powder manufactured from the same lot following each form of terminal sterilization. Each sample was prepared by dissolving 30±3 mg of powder in 50 mL of dilute aqueous hydrochloric acid (HCl) at pH 2 (n=12 for each group). 3-8% Tris Acetate gels were used and each well was filled to two thirds capacity. A 250 μg/mL type I collagen reference derived from bovine skin (the standard used in the Sircol collagen assay) was prepared and run in the far right lane of the gel (FIG. 34) to indicate where the alpha monomers, beta dimers, and gamma trimer of collagen were located down each lane. In the gel with E-Beam processed samples (FIG. 34, top panel), clear banding was visible at the position of the α1 and α2 monomers, faint bands were visible at the α1+α1 and α1+α2 dimers, and almost no banding was visible at the trimer location. Extensive smearing was seen throughout each lane, indicating widespread, nonspecific cleavage of proteins in the powder. In the powder samples processed with scCO₂ (FIG. 34, bottom panel), all major banding, including type I collagen monomers, dimers, and trimers were present and well defined. No smearing was present, and three other bands were visible at higher molecular weights. These higher molecular weight bands were also visible in the collagen reference lane.

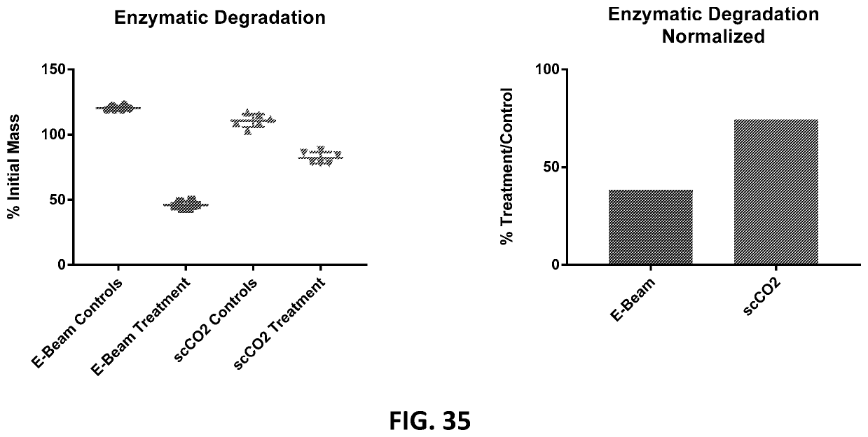

Resistance to Enzymatic Degradation: Three gel disc samples (diameter=12 mm, height=9 mm) were prepared for both powder sterilized with E-Beam and powder sterilized with scCO₂, using a powder concentration of 400 mg/3 mL PBS. Discs were cut into quarter sections to create a total of 12 samples. Six quarter sections from each group were used as control samples to be incubated in buffer solution (PBS with calcium and magnesium) without enzyme, and six were used as treatment samples, to be incubated in collagenase type I (200 activity units/mL) for two hours at 32° C. on a rocker table set to 100 rpm. Gel weights were taken before and after incubation, with the percent remaining weight serving as the primary metric to compare the groups. The mean (+SD) weight retention in control samples from powder processed with either E-Beam or scCO₂ sterilization was 120±2% and 111±5%, respectively (FIG. 35, left panel), suggesting that throughout the 2 hour incubation in buffer without enzyme, the gel quarter sections absorbed fluid from the surroundings in both groups. The mean E-Beam control sample retention was 9% greater than that of the scCO₂ group, a difference that was significant following an unpaired, two-tailed T test with Welch's correction (p=0.004). The mean (±SD) weight retention in treatment samples from powder processed with either E-Beam sterilization or scCO₂ was 46±3% and 82±4%, respectively. This difference was also statistically significant (p<0.001). The normalized weight retention of E-Beam gel samples (% Treatment E-Beam/Control E-Beam) was almost 2× less than that of scCO₂ samples at 38% and 74%, respectively (FIG. 35, right panel).

Mechanical Testing: A series of unconfined compression tests were performed to characterize the mechanical properties of the ECM-derived powder/PBS hydrogel. PBS is used as an isosmolar replacement for whole blood in this testing to isolate the mechanical contributions of gelation without the added contributions of clotting. Six samples were tested using powder from a single ECM-derived powder lot and sieve controlled to a particle size between 0.3 and 0.6 mm for each preparation method (200 mg E-Beam, 200 mg scCO₂, 400 mg E-Beam, or 400 mg scCO₂ powder vacuum mixed with 3 mL of PBS and incubated at 32° C. for over 1 hour).

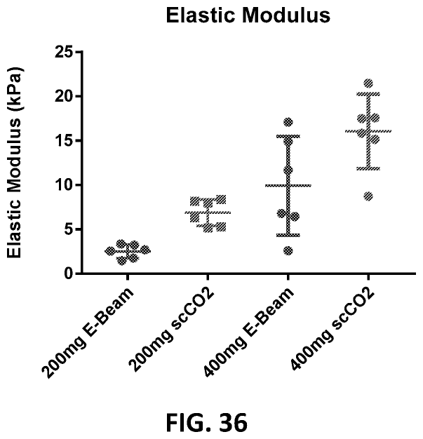

Elastic Modulus: The elastic modulus is a measure of stiffness that can be used to compare the resistance of gelled plugs to axial compression in the linear (elastic) region. Gel plugs were compressed at a uniform rate (0.05 mm/s) to a strain of 10% (roughly 0.8 mm). The modulus represents the ratio of stress (applied force/cross sectional area) divided by the strain (% length change compared to initial length). The mean (±SD) elastic moduli of gels prepared using a powder concentration of 200 mg/3 mL processed with either E-Beam sterilization or scCO₂ were 2.5±0.8 kPa and 6.9±1.5 kPa, respectively (FIG. 36). This 4.4 kPa difference was statistically significant according to an unpaired, two-tailed T test with Welch's correction with a p value of <0.001. The mean (±SD) elastic moduli of gels prepared using a powder concentration of 400 mg/3 mL processed with either E-Beam sterilization or scCO₂ were 9.9±5.6 kPa and 16.0±4.2 kPa, respectively. This 6.1 kPa difference was not statistically significant according to an unpaired, two-tailed T test with Welch's correction with a p value of 0.059.

In summary, the first portion of these studies were focused on the biochemical composition of ECM-derived powder that was sterilized with either 20-25 kGy electron beam radiation or supercritical carbon dioxide with an additive (NOVAKILL™; NovaSterilis, Inc., Lansing, NY). While the GAG content of each group was very similar, the measurable collagen content of the scCO₂ processed powder was significantly higher than the collagen content of the e-beam processed powder. While the phospholipid content of the ScCO₂ powder was significantly lower than that of the e-beam treated powder, the DNA content in each group was similar, suggesting no relevant differences in the breakdown of potentially antigenic cellular material. The second portion of these study focused on the physical properties of powder processed with either e-beam or scCO₂ when prepared as hydrogels at two powder concentrations: 200 mg and 400 mg in 3 mL PBS. Differences in resistance to enzymatic degradation as well as mechanical integrity as measured by stiffness (elastic modulus), resistance to fatigue (dynamic compression), viscous character (stress relaxation), and deformation (Poisson's ratio) were observed. For example, gels derived from scCO₂ sterilized powder were significantly more resistant to enzymatic degradation than gels derived from e-beam sterilized powder, retaining almost twice as much of their initial weight relative to controls incubated in buffer alone. The scCO₂ gels were also significantly stiffer, having a mechanical character that was less influenced by internal viscous flow in both concentrations, with elastic moduli over 1.5× that of e-beam gels and lower rates of stress relaxation over ten minutes. Dynamic compression testing revealed greater susceptibility of ScCO₂ gels to fatigue, however the stress response to achieve the same strain remained higher in scCO₂ gels at all time points in both concentrations. The strain-dependent Poisson ratio showed that the compressed gels derived from powder processed with either sterilization method responded with similar degrees of strain in the transverse plane. It was clear that the overall mechanical integrity of gels derived from scCO₂ sterilized powder was greater than that of gels derived from e-beam sterilized powder. The higher degree of protein preservation, especially type I collagen, as suggested by biochemical composition testing, likely allowed the ECM powder to maintain to a higher degree of its natural ability to undergo fibrillogenesis in an isosmolar environment, and through a higher degree of intermolecular interactions to maintain a more organized and mechanically resilient structure.

### Example 17—ACL Repair Study

Studies were conducted to compare the healing of a torn ACL using the established BEAR® scaffold (Miach Orthopedics, Westborough, MA) sterilized with electron beam irradiation or the mesodermal protein powder described herein. The mesodermal protein powder was terminally sterilized using scCO₂ at NovaSterilis (Lansing, NY). Before injection, 600 mg of the ECM-derived powder was placed into a syringe and mixed with 6 mL of fresh autologous blood from the surgical pig. Eight (n=8) adolescent (3 months old) male/female Yorkshire pigs underwent unilateral ACL transection to simulate ACL injury. Knee stability was restored with a suture stent and a suture was fixed to the tibial ACL stump to approximate it to the femoral stump. The ACL repair was aided by either a blood-soaked BEAR® scaffold in half of the animals (n=4) or mesodermal protein powder mixed with blood (n=4 animals). Groups were randomized to include an equal number of same sex animals and same side surgical knees. The animals were euthanized 6 weeks after the initial ACL repair procedure, and the repaired ACLs evaluated for their macroscopic and histologic appearance. Statistical comparisons between the two study groups were made using a student t-test, when assumptions of normality of data distribution and homogeneity of variance were met. A p-value of 0.05 was deemed statistically significant.

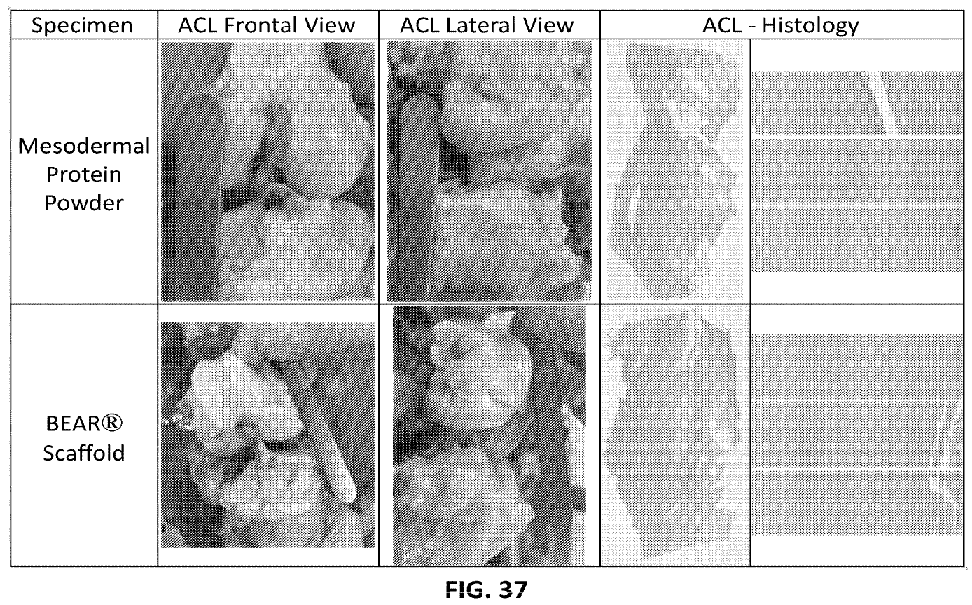

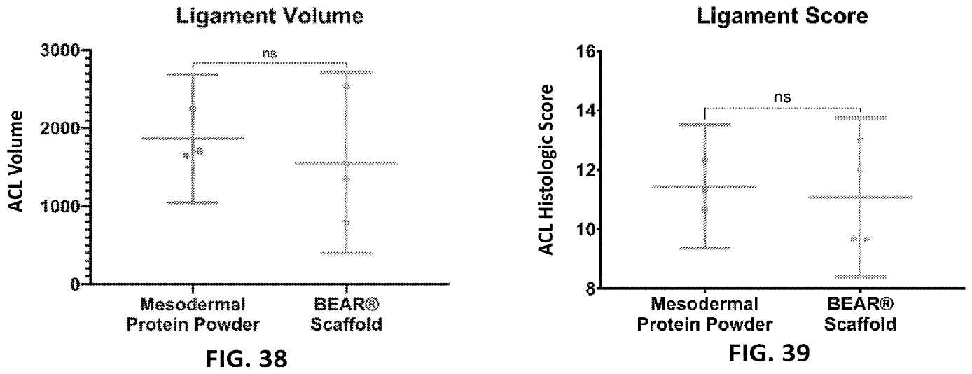

Six weeks after initial ACL repair, a successful reconnection between the femoral and tibial ACL stump was observed for all specimens (FIG. 37). No obvious differences in morphology or size were established between the ACLs repaired with the aid of either the mesodermal protein powder or the BEAR® Scaffold. Macroscopic measurements of the ACL volume showed no statistically significant difference between the ACLs repaired with the aid of either the mesodermal protein powder or the BEAR® scaffold (FIG. 38Error!Reference source not found.), and histological analysis of the repaired ACLs revealed no significant differences between the ACLs repaired with the aid of either the mesodermal protein powder or the BEAR® scaffold (FIG. 39). Thus, the use of either the mesodermal protein powder or the BEAR® scaffold to aid in the healing of ACL injures in a large animal model of ACL transection resulted in comparable scar tissue forming between the torn ends of the repaired ACL after 6 weeks, both on a macroscopic level and a microscopic level.

### Example 18—ACL Repair Study

A study was conducted to compare the healing of a torn rotator cuff tendon (RCT) using suture repair only, mesodermal protein powder, a mesodermal protein sheet, or the established BEAR® sheet. Twelve (n=12) adolescent (3 months old) male/female sheep underwent unilateral transection of the RCT followed in equal numbers (n=3) by suture repair only or treatment with mesodermal protein powder described herein, mesodermal protein sheet, or the established BEAR® sheet. The mesodermal protein powder was terminally sterilized using scCO₂ at NovaSterilis (Lansing, NY). Before injection, 600 mg of the mesodermal protein powder was placed into a syringe and mixed with 6 mL of fresh autologous blood from the surgical sheep. Groups were randomized to include an equal number of same sex animals and same side surgical knees. The animals were euthanized 8 weeks after the initial RCT repair procedure, and the shoulders were evaluated for their stability and range of motion and the repaired RCTs were evaluated for their macroscopic and histologic appearance. Statistical comparisons between the two study groups were made using a one-way-ANOVA, when assumptions of normality of data distribution and homogeneity of variance are met. A p-value of 0.05 was deemed statistically significant.

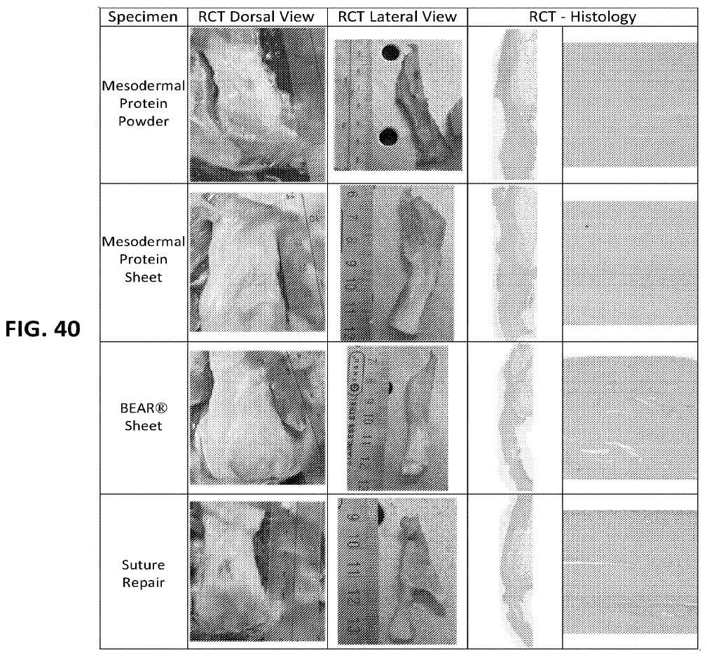

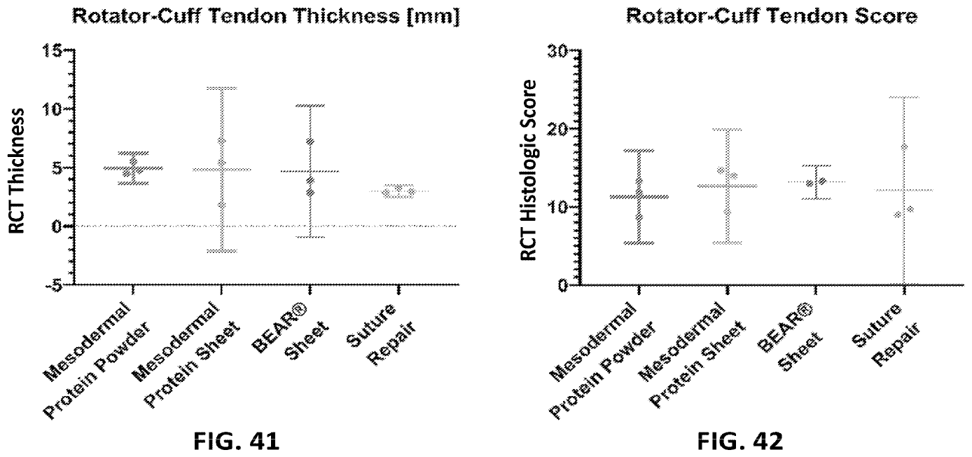

Eight weeks after initial RCT repair, a successful reconnection between the proximal and distal RCT stump was observed for all specimens (FIG. 40). Macroscopic measurements of the RCT thickness showed no statistically significant difference between the RCTs repaired with the aid of either suture repair only, mesodermal protein powder, mesodermal protein sheet, or the established BEAR® sheet (FIG. 41), and histological analysis of the repaired RCTs revealed no significant differences between the RCTs repaired with the aid of either suture repair only, mesodermal protein powder, mesodermal protein sheet, or the established BEAR® sheet (FIG. 42). The results of this pilot study indicate that the use of either suture repair only, mesodermal protein powder, mesodermal protein sheet, or the established BEAR® sheet to aid in the healing of RCT injures in a large animal model of RCT transection resulted in comparable scar tissue forming between the torn ends of the repaired RCTs after 8 weeks both on a macroscopic and microscopic level.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising a powdered extracellular matrix (ECM) component and a fluid, wherein the ECM component comprises mesodermal proteins comprising collagen, and wherein the concentration of the powdered ECM component in the fluid is about 50 mg/mL to about 200 mg/mL.

2. The composition of claim 1, wherein the concentration of the powdered ECM component in the fluid is about 100 to about 150 mg/mL.

3. The composition of claim 1, wherein the fluid is blood.

4. The composition of claim 1, wherein the powdered ECM component has an average particle size of about 0.1 mm to about 1 mm.

5. The composition of claim 1, further comprising a growth factor, platelets, white blood cells, stem cells, a cross-linker, a neutralizing agent, or any combination thereof.

6. The composition of claim 1, wherein the composition further comprises calcium.

7. The composition of claim 1, wherein the composition is substantially free of one or more of nucleic acid, glycosaminoglycan (GAG), phospholipid, active pepsin, and active virus.

8. A method for making a composition comprising a fluid and a powdered ECM component comprising mesodermal proteins that comprise collagen, wherein the method comprises:
providing a syringe containing the powdered ECM component, and
drawing an amount of the fluid into the syringe such that the concentration of the powdered ECM component in the fluid is about 50 mg/mL to about 200 mg/mL.

9. The method of claim 8, comprising drawing an amount of the fluid into the syringe such that the concentration of the powdered ECM component in the fluid is about 100 to about 150 mg/mL.

10. The method of claim 8, wherein the fluid is blood.

11. The method of claim 8, wherein the powdered ECM component has an average particle size of about 0.1 mm to about 1 mm.

12. The method of claim 8, wherein the ECM composition further comprises a growth factor, platelets, white blood cells, stem cells, a cross-linker, a neutralizing agent, or any combination thereof.

13. The method of claim 8, wherein the composition further comprises calcium.

14. The method of claim 8, wherein the composition is substantially free of one or more of nucleic acid, GAG, phospholipid, active pepsin, and active virus.

15. A method for treating a mammal, wherein the method comprises administering to a joint of a mammal that has or is at risk for developing arthritis at the joint an effective amount of a composition comprising a powdered ECM component and a fluid, wherein the ECM component comprises mesodermal proteins comprising collagen, and wherein the concentration of the powdered ECM component in the fluid is about 50 mg/mL to about 200 mg/mL.

16. The method of claim 15, wherein the concentration of the powdered ECM component in the fluid is about 100 to about 150 mg/mL.

17. The method of claim 15, wherein the fluid is blood.

18. The method of claim 15, wherein the powdered ECM component has an average particle size of about 0.1 mm to about 1 mm.

19. The method of claim 15, wherein the ECM composition further comprises a growth factor, platelets, white blood cells, stem cells, a cross-linker, a neutralizing agent, or any combination thereof.

20. The method of claim 15, wherein the composition further comprises calcium.

21. The method of claim 15, wherein the composition is substantially free of one or more of nucleic acid, GAG, phospholipid, active pepsin, and active virus.

22. The method of claim 15, wherein the mammal has an acute injury at the joint.

23. The method of claim 15, wherein the arthritis is osteoarthritis.

24. The method of claim 15, wherein the arthritis is post-traumatic arthritis.

25. The method of claim 24, wherein the post-traumatic arthritis is associated with an intra-articular injury or arthroscopic surgery.

26. The method of claim 25, wherein the intra-articular injury is selected from the group consisting of anterior cruciate ligament tear, anterior cruciate ligament rupture, meniscal injury, and cartilage injury.

27. The method of claim 15, wherein the mammal was surgically treated for a torn, fractured, strained, bruised, or ruptured intra-articular tissue at the joint at least one day prior to the administration of the composition.

28. The method of claim 15, wherein the joint is a joint of a hand, elbow, wrist, hip, knee, foot, shoulder, ankle, temporomandibular, or spine.

29. The method of claim 15, wherein the mammal has an injury associated with the development of arthritis.

30. The method of claim 15, wherein the administering comprises direct injection into the joint.

31. The method of claim 15, wherein the mammal is a human.

32. A method for treating a mammal having an intra-articular tissue defect, the method comprising, after visualization of the defect with an arthroscope, administering to the defect an effective amount of a composition comprising a powdered ECM component and a fluid, wherein the ECM component comprises mesodermal proteins comprising collagen, and wherein the concentration of the powdered ECM component in the fluid is about 50 mg/mL to about 200 mg/mL to the defect.

33. The method of claim 32, wherein the concentration of the powdered ECM component in the fluid is about 100 to about 150 mg/mL.

34. The method of claim 32, wherein the fluid is blood.

35. The method of claim 32, wherein the powdered ECM component has an average particle size of about 0.1 mm to about 1 mm.

36. The method of claim 32, wherein the ECM composition further comprises a growth factor, platelets, white blood cells, stem cells, a cross-linker, a neutralizing agent, or any combination thereof.

37. The method of claim 32, wherein the composition further comprises calcium.

38. The method of claim 32, wherein the composition is substantially free of one or more of nucleic acid, GAG, phospholipid, active pepsin, and active virus.

39. The method of claim 32, wherein the defect is an acute injury at the joint.

40. The method of claim 39, wherein the defect is selected from the group consisting of anterior cruciate ligament tear, anterior cruciate ligament rupture, meniscal injury, and cartilage injury.

41. The method of claim 32, wherein defect is an injury associated with the development of arthritis.

42. The method of claim 32, wherein the administering comprises direct injection into the defect.

43. The method of claim 32, wherein the mammal is a human.

44. A method for making a powdered composition comprising mesodermal extracellular matrix (ECM) proteins, the method comprising:
decellularizing a tissue sample comprising tissue arising from mammalian mesoderm;
treating the tissue sample, before or after decellularization, with a composition comprising peracetic acid;
freeze-drying the decellularized tissue sample; and
milling the freeze-dried tissue into a powder.

45. The method of claim 44, wherein the composition comprising peracetic acid comprises about 0.1% peracetic acid.

46. The method of claim 44, comprising treating the tissue sample, before or after decellularization, for about 5 to 30 minutes with the composition comprising peracetic acid.

47. The method of claim 44, wherein the composition comprising peracetic acid further comprises hydrogen peroxide.

48. The method of claim 47, wherein the composition comprises about 1% hydrogen peroxide.

49. The method of claim 44, further comprising, prior to the freeze-drying, treating the decellularized tissue sample with an enzyme, thereby removing species-specific ends of collagen molecules.

50. The method of claim 44, further comprising treating the powder with supercritical carbon dioxide (scCO₂).

51. The method of claim 44, wherein the powder has an average particle size of about 0.1 mm to about 1 mm.

52. The method of claim 44, wherein the composition further comprises a growth factor, platelets, white blood cells, stem cells, a cross-linker, a neutralizing agent, or any combination thereof.

53. The method of claim 44, wherein the composition further comprises calcium.

54. The method of claim 44, wherein the composition is substantially free of one or more of nucleic acid, GAG, phospholipid, active pepsin, and active virus.

55. A method for making a powdered composition comprising mesodermal ECM proteins, the method comprising:
decellularizing a tissue sample comprising tissue from mammalian mesoderm;
freeze-drying the decellularized tissue sample;
milling the freeze-dried tissue slurry into a powder; and
treating the powder with scCO₂.

56. The method of claim 55, further comprising, prior to the freeze-drying, treating the decellularized tissue sample with an enzyme, thereby removing species-specific ends of collagen molecules.

57. The method of claim 55, further comprising treating the tissue sample, before or after decellularization, with a composition comprising peracetic acid.

58. The method of claim 57, wherein the composition comprising peracetic acid comprises about 0.1% peracetic acid.

59. The method of claim 57, comprising treating the tissue sample, before or after decellularization, for about 5 to 30 minutes with the composition comprising peracetic acid.

60. The method of claim 57, wherein the composition comprising peracetic acid further comprises hydrogen peroxide.

61. The method of claim 60, wherein the composition comprises about 1% hydrogen peroxide.

62. The method of claim 55, wherein the powder has an average particle size of about 0.1 mm to about 1 mm.

63. The method of claim 55, wherein the composition further comprises a growth factor, platelets, white blood cells, stem cells, a cross-linker, a neutralizing agent, or any combination thereof.

64. The method of claim 55, wherein the composition further comprises calcium.

65. The method of claim 55, wherein the composition is substantially free of one or more of nucleic acid, GAG, phospholipid, active pepsin, and active virus.

66. A method for making a composition comprising blood and a powdered mesodermal ECM component comprising collagen, wherein the method comprises:
providing a syringe containing the powdered ECM component;
contacting a sample of blood with an anticoagulant;
drawing an amount of the blood into the syringe containing the powdered ECM component such that the concentration of the powdered ECM component in the blood is about 50 mg/mL to about 200 mg/mL; and
adding a calcium chloride solution to the syringe, thereby deactivating the anticoagulant.

67. The method of claim 66, comprising drawing an amount of the blood into the syringe such that the concentration of the powdered ECM component in the blood is about 100 to about 150 mg/mL.

68. The method of claim 66, wherein the powdered ECM component has an average particle size of about 0.1 mm to about 1 mm.

69. The method of claim 66, wherein the ECM composition further comprises a growth factor, platelets, white blood cells, stem cells, a cross-linker, a neutralizing agent, or any combination thereof.

70. The method of claim 66, wherein the composition is substantially free of one or more of nucleic acid, GAG, phospholipid, active pepsin, and active virus.

71. The method of claim 66, wherein the calcium chloride solution has a concentration of about 35 mM to about 45 mM.

72. The method of claim 66, comprising adding the calcium chloride solution to the syringe to obtain a mixture comprising a 1:9 ratio of calcium chloride solution to blood.
